SEQUENCE LISTING (10 sequences)

SEQ ID NO: 1 - DNA, 21 bp, Artificial Sequence
  Synthetic oligonucleotide
tgcgtcctgcacaactgtatc

SEQ ID NO: 2 - DNA, 20 bp, Artificial Sequence
  Synthetic oligonucleotide
ccaggaacgtgtgaccatca

SEQ ID NO: 3 - DNA, 19 bp, Artificial Sequence
  Synthetic oligonucleotide
ctgaaggaccctgcgaact

SEQ ID NO: 4 - DNA, 21 bp, Artificial Sequence
  Synthetic oligonucleotide
tcgctcaaaccagcctttcag

SEQ ID NO: 5 - DNA, 20 bp, Artificial Sequence
  Synthetic oligonucleotide
actccctggagatgactccc

SEQ ID NO: 6 - DNA, 20 bp, Artificial Sequence
  Synthetic oligonucleotide
tcttgcactttgttgctggc

SEQ ID NO: 7 - DNA, 23 bp, Artificial Sequence
  Synthetic oligonucleotide
atatggcaagaaggtgatggtcc

SEQ ID NO: 8 - DNA, 21 bp, Artificial Sequence
  Synthetic oligonucleotide
gggcttgtcctaacaaagctg

SEQ ID NO: 9 - DNA, 18 bp, Artificial Sequence
  Synthetic oligonucleotide
ctcgcctttgccgatccg

SEQ ID NO: 10 - DNA, 20 bp, Artificial Sequence
  Synthetic oligonucleotide
tctccatgtcgtcccagttg

(19) United States
(12) Patent Application Publication
(10) Pub. No.: US 2024/0277767 A1
(43) Pub. Date: Aug. 22, 2024

(54) METHODS AND USES

(71) Applicant: PROLEUKOCYTE AB, Rönninge (SE)

(72) Inventors: Stina Linnéa WICKSTRÖM, Rönninge (SE); Rolf KIESSLING, Stockholm (SE); Elias Set Jenö ARNÉR, Lidingö (SE)

(73) Assignee: PROLEUKOCYTE AB, Rönninge (SE)

(21) Appl. No.: 18/556,484

(22) PCT Filed: Apr. 27, 2022
(86) PCT No.: PCT/EP2022/061216

§ 371 Date: Apr. 30, 2024

(30) Foreign Application Priority Data
Apr. 28, 2021 (GB) 2106040.5

(51) Int. Cl. A61K 35/17; A61P 35/00; C12N 5/00; C12N 5/0783

(52) U.S. Cl. CPC A61K 35/17; A61P 35/00; C12N 5/0018; C12N 5/0636; C12N 5/0646; C12N 2501/2302

(57) ABSTRACT

The present invention relates to uses of and methods of using activators of Nrf2 to enhance natural killer (NK) cell and/or T cell activity and/or survival, particularly in response to stress. The NK cells and/or T cells can be utilised in the treatment of cancer via enhanced cell therapy.

### METHODS AND USES

The present invention relates to uses of and methods of using activators of Nrf2 to enhance natural killer (NK) cell and/or T cell activity and/or survival, particularly in response to stress. The NK cells and/or T cells can be utilised in the treatment of cancer and tumours via enhanced cell therapy.

Immunotherapy of malignant diseases is a rapidly expanding field and increases the therapeutic options available for cancer patients. In particular, adoptive cell therapy (ACT) is a potent method, which involves the transfer of immune cells into a patient. The immune cells may originate from the patient themselves (autologous cells) or may be obtained from a donor (allogenic). ACT typically involves extraction of immune cells from the patient or donor, followed by manipulation (for example by treatment with external factors, genetic modification or expansion in numbers) before they are (re)introduced to the patient. ACT includes, but is not limited to, therapy based on peripheral blood mononuclear cells (PBMCs) engineered to become tumour specific; on expansion of tumour-infiltrating lymphocytes (TIL) cultured from a surgical resection of the tumour; on mononuclear cells from another tissues; and/or on expansion of lymphocytes infiltrating other tissues (e.g. the lymph nodes).

ACTs using T cells (including tumour infiltrating lymphocytes), NK cells or genetically modified NK or T cells (such as chimeric antigen receptor (CAR)-T cells or CAR-NK cells) have proven to be very effective as immunotherapies to treat patients with various advanced malignancies. For example, clinical trials have shown promising results with TIL therapy of malignant melanoma yielding an overall response (OR) rate around 30-50%. T cells engineered to express T cell receptors (TCRs) specific for tumour antigens in solid tumours have shown a clinical response with an OR rate of 45-70%. ACT with chimeric antigen receptor (CAR) T cells engineered to express CD19 for treatment of relapsing B-cell ALL were recently FDA approved (ClincalTrials.gov ID: NCT02435849). CAR-based ACT have seen complete responses (CRs) ranging from 68-100% for adult and paediatric B cell malignancies in multiple independent clinical trials.

Regardless of the promising results, there are still hurdles to overcome and room for improvement in existing adoptive cell therapies. There is a high level of commercial interest in finding ways to enhance existing cell therapies against cancer and/or in finding ways of using cell therapies to treat solid tumours, which are often not sensitive to these types of cell therapy.

The tumour microenvironment (TME) is a hostile environment and possesses variety of stresses including oxidative stress, hypoxia, starvation (including nutrient and/or oxygen limitation), mechanical stresses. The viability and activity of NK cells and T-cells can be suppressed by the cancer cells and/or the TME, which decreases their efficacy in ACT. Cancer cells and tumour-infiltrating immune cells, such as myeloid-derived suppressor cells (MDSC), contribute to creating this hostile and immune suppressive TME, especially by the production of reactive oxygen species (ROS) (Schmielau and Finn, 2001).

Tumor infiltrating lymphocytes are associated with improved prognosis and progression-free survival in cancer patients undergoing immunotherapy such as the use of immune checkpoint inhibitors (ICIs) against CTLA-4 and PD-1/PD-L1. However, still only a fraction of patients has a durable long-term response to such therapies as many other factors seem to be involved in the tumor microenvironment in the down regulation of the immune response. One of the key factors seems to be exhaustion of T cells resulting in the physical elimination and/or dysfunction of antigen-specific T cells. Factors involved in this exhaustion phenomenon involve surface markers expressed on tumor cells, lymphoid and mononuclear cells and soluble molecules secreted from regulatory T cells and NK cells in the tumor microenvironment (TME). But, also the lack of stimulatory factors such as interferon gamma and IL-2 is evident in the TME.

Reversal of T-cell exhaustion is a key target in the development of new classes of Immune Checkpoint Inhibitors (“ICIs”) either as a monotherapy or in combination with already established therapies. However, as these targets often are also responsible for inducing immune tolerance, avoiding autoimmune responses, systemic administration of inhibitors can cause serious side effects. In addition, administering T-cell stimulatory molecules such as IL-2 can also cause serious and sometimes fatal side effects and therefore needs to be managed by skilled clinicians. Some approaches have been taken to administer drug candidates locally into the tumor thereby possibly avoiding systemic side effects. However, as cancer cells are distributed all over the body in many metastatic patients, the likelihood of this approach to be successful under such circumstances can be questioned.

The use of tumor infiltrating lymphocyte (TIL) therapy has shown significant clinical benefit with objective response rates of up to 55% and complete responses in up to 20% of patients in various malignancies such as metastatic melanoma, head and neck and cervical cancer. In short, this kind of therapy leverages the in vitro expansion of autologous T lymphocytes by stimulating fragments from the excised tumor with IL-2, anti-CD3 antibodies and feeder cells and thereby growing these cells to billions before re-administering the T cells in back to the lymphodepleted patient followed by IL-2 infusion where after regression of the tumor is promoted.

TIL therapy is costly and takes time. It would therefore be advantageous to optimize the current methods and identify ways to shorten the duration for expansion of the TILs, increase the expansion rate, and also achieve more favorable phenotypes and functionality.

Reactive Oxygen Species (ROS) are chemically reactive oxygen-containing molecules, such as superoxide radicals (O₂⁻), hydroxyl radicals (OH) or hydrogen peroxide (H₂O₂). Intracellularly produced ROS play an important role as secondary messengers in cellular signalling cascades (Schieber and Chandel, 2014; Sies and Jones, 2020). The reduction-oxidation (redox) balance regulates many biological processes, including immune responses (Mehrotra et al. 2009). There are two major reductive enzyme systems maintaining redox homeostasis, namely the glutathione (GSH) and Thioredoxin (Trx) systems, utilizing NADPH as reducing equivalent (Miller et al. 2018).

Bursts of ROS produced by NADPH oxidases are essential to mediate innate immune cell functions against invading microbes and as anti-tumoural response (Sies and Jones, 2020). However, sustained elevated ROS levels have been shown to diminish the immune response by inducing poor effector functions or cell death in T and NK cells (Norell et al., 2009; Harlin et al., 2007). Early studies showed that ROS produced by autologous monocyte in the TME suppressed NK- and T cell function and their capability to response to cytokine activation (Hellstrand, 2002). Various studies have shown that increased ROS levels leads to downregulation of the TCR/CD3 complex in T cells and the low-affinity Fc receptor FcγRIII (also known as CD16) on NK cells, resulting in reduced cytotoxic capacity (Otsuji et al. 1996; Kono et al. 1996). Furthermore, ROS produced by tumour-derived macrophages, granulocytes or phagocytes can reduce NK- and T-cell activity by inducing downregulation of the CD16ζ- and CD3ζ-chain, respectively, as well as NKp46 and NKG2D downregulation.

Hypoxia, one of the key hallmarks of cancer, compromises anti-cancer immune responses, particularly via reducing the survival, cytolytic and migratory activity of effector cells such as CD4+ cells, CD8+ cytotoxic T cells, natural killer-like T cells and natural killer cells (Multhoff and Vaupel, 2020). Specifically, hypoxia and also starvation can lead to exclusion of lymphocytes, particularly T cells, from the tumour site and can result in limitations on the efficiency of cell immunotherapy of solid tumours (Pietrobon and Marincola 2021). The nutrient and oxygen deprived tumour microenvironment leads to suppression of NK cell activity and effector function, which may be further worsened by the presence of tumour-derived metabolic end products such as lactate (Terrén et al., 2019).

One way to combat this deleterious effect on the immune system is to counteract and/or reduce the accumulation of ROS in the TME. It has been shown that addition of histamine can inhibit ROS production by the monocytes and thereby protect NK cells and T cells from ROS mediated suppression. However, this approach is not without drawbacks. For instance, reducing the accumulation of ROS in the TME can have a negative effect on the efficacy of anti-cancer therapies such as radiation and/or chemotherapy. The ROS induced by these therapies directly kill cancer cells, which are often sensitive to ROS, and therefore reduction of ROS in order to protect NK and/or T cells from suppression may result in less efficient treatment by conventional anti-cancer therapies.

Another disadvantage of treatments aimed at reducing accumulation of ROS in the TME in order to prevent suppression of immune cells is that they are not specific. This can lead to the inhibition of myeloid-derived suppressor cells (MDSC) and/or the targeting of other undesirable signalling pathways within the target cells and/or other cells in the patient.

Against this background, the present inventors have surprisingly discovered that contacting NK and/or T cells with an activator of Nrf2 results in enhanced NK and/or T cell activity and/or survival in response to stress, thereby allowing them to better tolerate and survive in environments of stress, particularly those within the tumour microenvironment.

Particularly surprisingly, the present inventors have discovered that contact with an Nrf2 activator results in enhanced activity and/or survival of NK and/or T cells for a defined period of time which is beneficial and compatible with ex vivo therapy. Specifically, the time-period of enhanced activity and/or survival is sufficiently long to allow the NK and/or T cells to be contacted ex vivo and then introduced into a patient, and then persists for a sufficient period to have a beneficial therapeutic effect in the patient. Subsequently, the enhanced activity and/or survival of the NK and/or T cells reduces and returns to normal levels, which advantageously prevents long-term or persistent immune stimulation in the patient (which could have adverse consequences).

Thus, the present inventors have surprisingly developed a therapeutic approach in which NK and/or T cell activity and/or survival is enhanced, and is surprisingly compatible with ex vivo therapies; and additionally provides a defined time-period of enhancement which allows the period of effective immunotherapy to be controlled.

In a first aspect, the invention provides the use of an activator of Nrf2 for: preventing and/or reducing the suppression of NK cell and/or T cell activity caused by one or more stress; and/or inducing and/or maintaining and/or increasing NK cell and/or T cell activity in the presence of one or more stress; and/or increasing NK cell and/or T cell survival in response to one or more stress.

Thus, the Nrf2 activator can be used for preventing the suppression of NK cell and/or T cell activity caused by one or more stress. The Nrf2 activator can be used for reducing the suppression of NK cell and/or T cell activity caused by one or more stress. The Nrf2 activator can be used for inducing NK cell and/or T cell activity in the presence of one or more stress. The Nrf2 activator can be used for maintaining NK cell and/or T cell activity in the presence of one or more stress. The Nrf2 activator can be used for increasing NK cell and/or T cell survival in response to one or more stress. The Nrf2 activator can be used for increasing NK cell and/or T cell survival in response to one or more stress.

In a second aspect, the invention provides a method for: preventing and/or reducing the suppression of NK cell and/or T cell activity caused by one or more stress; and/or inducing and/or maintaining and/or increasing NK cell and/or T cell activity in the presence of one or more stress; and/or increasing NK cell and/or T cell survival in response to one or more stress;
wherein the method comprises the step of contacting one or more NK cell and/or T cell with an activator of Nrf2.

In another embodiment, the invention provides the use of an activator of Nrf2 for: preventing and/or reducing the suppression of NK cell and/or T cell activity in a tumour and/or cancer; and/or inducing and/or maintaining and/or increasing NK cell and/or T cell activity in a tumour and/or cancer; and/or increasing NK cell and/or T cell survival in a tumour and/or cancer.

In another embodiment, the invention provides a method for: preventing and/or reducing the suppression of NK cell and/or T cell activity in a tumour and/or cancer; and/or inducing and/or maintaining and/or increasing NK cell and/or T cell activity in the presence in a tumour and/or cancer; and/or increasing NK cell and/or T cell survival in a tumour and/or cancer; wherein the method comprises the step of contacting one or more NK cell and/or T cell with an activator of Nrf2.

As shown in the accompanying Examples, the present inventors have demonstrated that activators of Nrf2, such as auranofin, sulforaphane and/or dimethyl fumarate, enhances NK and/or T cell activity, particularly when the cells are subjected to stress, such as oxidative stress caused by ROS. Contacting NK and/or T cells with auranofin renders these cells more resistant to hydrogen peroxide or monocyte derived ROS, resulting in increased tumour elimination and cytokine release.

The inventors have therefore demonstrated that, by targeting the intrinsic redox systems and detoxification systems via Nrf2 in immune cells, the resistance of NK and/or T cells towards ROS and other stresses can be increased. That targeting of intrinsic redox pathways therefore represents a new general strategy for generating NK and/or T cells with improved anti-tumour capacity and/or resistance to stress, and thereby improve adoptive cell therapies.

By “activator of Nrf2” we include agents that activate and/or increase one or more activity of Nrf2 (i.e. “nuclear factor E2-related factor 2”).

Nrf2 is a transcription factor that is regulated by the constitutively expressed Keap1 (Kelch-like ECH-associated protein1). In non-stressed cells, the Kelch domains of Keap1 bind to Nrf2 and promotes its degradation by the 26S proteasome. During targeting of Keap1 with electrophiles, upon oxidative stress, or upon inhibition of thioredoxin reductase 1 (TrxR1), Keap1 loses the capacity to tightly bind Nrf2, thereby allowing Nrf2 to enter the nucleus (Cebula et al., 2015; Lei et al., 2016). Activated Nrf2 binds to antioxidant response elements (ARE) in target gene promoters thereby initiating transcription of genes encoding detoxifying enzymes, cytoprotective proteins and/or antioxidant proteins such as NAD(P)H quinione oxidoreductase 1 (NQO1), glutathione S transferases (GST) or heme oxygenase 1 (HO-1). The “activator of Nrf2” may activate and/or increase one or more activity of Nrf2 via TrxR1 inhibition and/or via Keap1 inhibition.

Thus the term “activator of Nrf2” includes agents that activate and/or increase Nrf2-mediated gene expression; for example, inducing and/or increasing the level of expression of one or more Nrf2-controlled gene; and/or inducing and/or increasing the number of Nrf2-controlled genes that are expressed. Those skilled in the art will be aware of Nrf2-controlled genes and how to identify others, and will be aware of methods for determining gene expression and detecting an increase in the level and/or number of genes expressed by the Nrf2 transcription factor. The Nrf2-controlled gene may encode any of the proteins that are affected at constitutive expression levels by Nrf2 knockout e.g. any of those proteins identified in Kitteringham et al. 2010, which is hereby incorporated by reference. Preferably, the Nrf2-controlled genes may be selected from: NQO1, and/or HO1, and/or TrxR1, and/or GST genes, and/or HMOX and/or carbonyl reductase.

Where the activator of Nrf2 induces and/or increases the level of expression of one or more Nrf2-controlled gene, that may be an increase of about 10%, or about 20%, or about 30%, or about 40%, or about 50%, or about 60%, or about 70%, or about 80%, or about 90%, or about 100%, or about 200%, or about 300%, or about 400%, or about 500%, or about 1000%, or about 1500%, or about 2000%, or about 3000%, or about 5000%, or about 10000%, or about 20000% or about 30000% or more; relative to the level of expression of the one or more Nrf2-controlled gene in the absence of the activator of Nrf2.

Where the activator of Nrf2 induces and/or increases the level of expression of one or more Nrf2-controlled gene, that may be an increase of about 1-fold, or about 5-fold, or about 10-fold, or about 25-fold, or about 50-fold, or about 75-fold, or about 100-fold, or about 200-fold, or about 300-fold, or about 400-fold, or about 500-fold, or about 600-fold, or about 700-fold, or about 800-fold, or about 900-fold, or about 1000-fold, or about 1250-fold, or about 1500-fold, or about 1750-fold, or about 2000-fold, or about 2500-fold, or about 3000-fold or more; relative to the level of expression of the one or more Nrf2-controlled gene in the absence of the activator of Nrf2.

In an embodiment, the activator of Nrf2 may increase the expression of one or more Nrf2-controlled genes at the messenger RNA level by between 1 and 3000-fold. In an embodiment, the activator of Nrf2 may increase the expression of one or more Nrf2-controllwed genes at the protein product level by between 1% and 100%, preferably between 20% and 60%.

Where the activator of Nrf2 induces and/or increases the number of Nrf2-controlled genes that are expressed, that may be an increase of about 1, or about 2, or about 3, or about 5, or about 10, or about 20, or about 30, or about 40, or about 50, or about 60, or about 70, or about 80, or about 90, or about 100, or about 200, about 300, about 400, about 500 or more genes, relative to the number of Nrf2-controlled genes that are expressed in the absence of the activator of Nrf2.

The activator of Nrf2 may decrease and/or inhibit and/or suppress expression of ATP-citrate lyase.

By “expression” we include the transcription of one or more of Nrf2-controlled gene into messenger RNA (mRNA) and/or the production of one or more gene product(s), particularly protein product(s), encoded by one or more Nrf2-controlled gene.

Level of expression can be determined by any suitable technique in the art, for example by measuring and/or quantifying mRNA levels or by measuring and/or quantifying levels of the protein product encoded by the Nrf2-contolled gene. Suitable methods may include: qPCR; northern blotting; serial analysis of gene expression (SAGE); microarray analysis; reverse transcription polymerase chain reaction (RT-PCR); real-time quantitative reverse transcription PCR (qRT-PCR); staining the protein product and/or mRNA; RNA seq; fluorescence in situ hybridisation (FISH); Western blotting; SDS-PAGE analysis; reporter gene assays; and/or any other suitable technique.

The activation of Nrf2 may be measured using Nrf2 activation kits, such as Nrf2 Transcription Factor Assay Kit (Colorimetric) supplied by Abcam (Product number: ab207223) and/or by the methods outlined in Dikovskaya et al. 2019.

As discussed above, Nrf2 is regulated by other cellular proteins, such as thioredoxin reductase and/or Keap1. In an embodiment, the activator of Nrf2 activates Nrf2 directly. In an alternative embodiment, the activator of Nrf2 activates Nrf2 indirectly, for example via inhibition of thioredoxin reductase (TrxR) and/or via inhibition of Keap1. Therefore, in some instances the activator of Nrf2 may also be an inhibitor of TrxR and/or an inhibitor of Keap1.

In a preferred embodiment, the activator of Nrf2 is one or more activator selected from the group comprising: auranofin (triethylphosphine gold), dimethyl fumarate (DMF), sulforaphane, curcumin, resveratrol, naringenin and/or agmatine. In alternative embodiments, the activator of Nrf2 is one or more activator selected from the group consisting of: caffeic acid phenethyl ester, methylglyoxal, itaconate, tertiary butylhydroquinone (tBHQ), Zn²⁺, 15-deoxy-Δ12, 14-prostaglandin J2, 1,2-naphthoquinone, cadmium chloride, methyl-mercury, arsenic, diethylmaleate, dexamethasone (DEX), Bardoxolone-methyl (CDDO-Me), RTA-408 (omaveloxolone), ALKS-8700, Oltiprazm Ursodiol, Sulforadex (SFX-01), ITH12674, CXA-10, any of the peptide inhibitors listed in Table 2 of Robledinos-Antón et al. 2019, cinnamic acid ester, cinnamic aldehyde, flavonols (e.g. Myricetin, Quercetin), flavan-3-ols (e.g. Catechin, Epicatechin, Epicatechin gallate, Epigallocatechin gallate), flavanonols (e.g. Taxifolin), hydroxyalkenals (e.g. 4-Hydroxynonenal), prostaglandins (e.g. 15d-PGJ2z, Prostaglandin A1), quinones (e.g. Juglone, Naphthazarin, PQQ Cofactor), polyphenols (e.g. Ellagic acid); benzene derivatives (e.g. Dinitrochlorobenzene (CDNB, DNCB), Dinitrofluorobenzene (FDNB, DNFB)), isothiocyanates (e.g. Sulforaphane, Benzyl isothiocyanate (BITC), phenethyl isothiocyanate (PEITC), allyl isothiocyanate (AITC)), arsenic compounds (e.g. Arsenite (AsIII), Monomethylarsonous acid, Arsenate (AsV), Arsenic trioxide, Phenylarsine oxide), gold compounds (e.g. Auranofin, Aurothiomalate, Gold (III) chloride), platinum compounds (e.g. Cisplatin, Nedaplatin, Oxaliplatin), gadolinium compound (e.g. Motexafin gadolinium); mercury compounds (e.g. Mercuric chloride, Monomethylmercury), sulphur mustard (2-chloroethyl ethyl sulfide); nitrosourea (e.g. Carmustine (BCNU)), acetaminophen metabolite (e.g. NAPQI), Schistosomicide (e.g. Oltipraz), statins (e.g. Simvastatin, Fluvastatin), unsaturated aldehyde (e.g. Acrolein), nitrogen mustard (e.g. Chlorambucil, Melphalan), and/or Dimethyl (E)-butenedioate. The activator or Nrf2 may be a non-conding RNA, such as a mircroRNA, that allows for RNA silencing of Keap1 and/or TrxR1.

In a preferred embodiment, the activator of Nrf2 is auranofin.

Auranofin (AUF) is a gold (I)-containing phosphine compound that was approved in 1985 to treat patients with Rheumatoid arthritis (Madeira et al., 2012). Auranofin is a strong activator of Nfr2, presumably via inhibition of TrxR (Saei et al., 2020), which can be attributed to the high reactivity of AUF with TrxR.

High doses of AUF have been shown to be toxic and induce cell death in cancer cells (including cancer cells), hence the usage of AUF as anti-cancer therapy in clinical trials (Stafford et al., 2018; Rigobello et al., 2008).

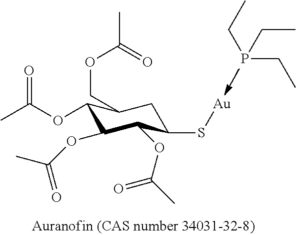

Dimethyl fumarate (also known as DMF, Dimethyl (E)-butenedioate, Tecfidera) is the methyl ester of fumaric acid. It is currently approved by the FDA for the treatment of multiple sclerosis and by the European Medicines Agency (EMA) for use in psoriasis.

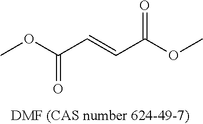

Sulforaphane (also known as sulphoraphane and 1-Isothiocyanato-4-(methanesulfinyl)butane) is a member of the isothiocyanate group of organosulphur compounds, and is found in many cruciferous vegetables, including broccoli.

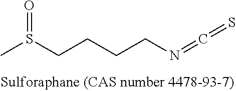

Curcumin, resveratrol, naringenin and agmatine are known to function as Nrf2 activators (Yang et al. 2020).

By “stress”, we include factors and/or conditions that are harmful to the one or more T cells and/or NK cell, in that they inhibit and/or suppress NK and/or T cell activity or survival. Exemplary activities of NK and/or T cells that are inhibited and/or suppressed by such stress are discussed herein.

In an embodiment, the reduction of the NK and/or T cell activity caused by stress may be a reduction of about 10%, or about 20%, or about 30%, or about 40%, or about 50%, or about 60%, or about 70%, or about 80%, or about 90%, or about 100% relative to the level of that activity in the absence of the stress. Those skilled in the art will be aware of suitable methods for determining the NK and/or T cell activities discussed herein.

In an embodiment, the reduction of the NK and/or T cell survival caused by stress may be a reduction of about 10%, or about 20%, or about 30%, or about 40%, or about 50%, or about 60%, or about 70%, or about 80%, or about 90%, or about 100% of the number of NK and/or T cells in a given period of time, relative to the number in the absence of the stress. Those skilled in the art will be aware of suitable methods for determining the NK and/or T cell survival.

In an embodiment the one or more stress is in a tumour microenvironment and/or is in the peripheral blood and/or organ of a cancer patient.

By “tumour microenvironment” (TME), we include the environment in and/or around a tumour, which may include one or more of the following: cancer/tumour cells; surrounding blood vessels (e.g. vascular endothelial cells); stromal tissue; pericytes; immune cells (e.g. natural killer (NK) cells, T lymphocytes, B lymphocytes, myeloid cells, tumour-associated macrophages (TAMs), myeloid-derived suppressor cells (MDSCs), tumour-associated neutrophils (TANs), terminally-differentiated myeloid dendritic cells, macrophages); cancer-associated fibroblasts; signalling molecules and/or the extracellular matrix (ECM).

In an embodiment the one or more stress is oxidative stress, hypoxia, reoxygenation and/or starvation. In a most preferred embodiment, the stress is oxidative stress.

By “oxidative stress”, we include the presence of reactive oxygen species (ROS), particularly the presence of a higher level of ROS than is present in normal and/or healthy environment and/or cell and/or tissue. By “oxidative stress”, we further include when there is an imbalance between the level of ROS and the ability to neutralise or detoxify the ROS, which leads to a redox imbalance and/or leads to higher ROS levels than is present in normal and/or healthy environment and/or cell and/or tissue. The skilled person would understand and/or be able to determine what level of ROS is typically present in any given normal and/or healthy environment and/or cell and/or tissue.

Oxidative stress and/or ROS may be measured by any suitable method known in the art, including by direct detection of ROS, by indirect detection of ROS (such as by assessing RNA/DNA damage and/or upregulation of genes) and/or by detection of glutathione (GSH) levels. ROS may be detected directly using flow cytometry, microscopy or live imaging (e.g. Incucyte™ instrument by Sartorius) by employing fluorescent dyes that oxidase in the presence of ROS and/or reagents that are specific for one or more ROS. Luminescence, lipid peroxidation and/or microplate analysis can also be used. ROS can be detected by measuring reduced glutathione (GSH) levels, which provides an indication of redox potential of a cell and/or a cell's ability to prevent oxidative stress. Intracellular probes may be used for the sensitive detection and localisation of GSH, which may be analysed by flow cytometry. ROS and oxidative stress may be measured indirectly by measuring RNA/DNA damage, upregulation of genes associated with ROS and oxidative stress and/or by measuring the presence of oxidated proteins, lipids or DNA molecules in the circulation. For example, 8-hydroxydeoxyguanosine (8-OHdG) may be used as a marker for DNA damage and/or oxidative stress. Comet assays, assays for apurinic/apyrimidinic sites, and assays for aldehyde-induced damage can be used to measure DNA damage related to oxidative stress and ROS.

By “hypoxia”, we include low oxygen conditions. Hypoxia may result from an imbalance between oxygen delivery and oxygen consumption. Hypoxia is a common feature of solid tumours, and hypoxic regions may develop due to aberrant blood vessel formations; fluctuations in blood flow; and/or increasing oxygen demands from rapid tumour expansion.

Hypoxia in cells and/or tissue may be measured by any suitable method, including using commercially-available kits for fixed cells or live cells comprising fluorescent probes, as are well known in the art. Hypoxia may also be detected by upregulation of relevant genes, such as HIF1a. The skilled person would understand that normal oxygen levels may be tissue-dependent and would be able to determine what hypoxic conditions are for a given tissue or cell based on the prior art and/or routine experimentation.

By “reoxygenation”, we include reoxygenation or exposure to oxygen and/or increased levels of oxygen after a period of hypoxia. Reoxygenation may be measured using the same kits as discussed above for measuring hypoxia, as are well known to those in the art. A sample from hypoxic conditions may be used as a reference.

By “starvation”, we include an environment with low levels of nutrient availability and/or nutrient-limiting conditions. Nutrients may include, but are not limited to, amino acids (particularly tryptophan and arginine), zinc, magnesium, selenium, vitamins, folic acid and/or constituents of albumin. By “low levels” we include less than 90%, or 80%, or less than 70%, or less than 60%, or less than 50%, or less than 40%, or less than 30%, or less than 20%, or less than 10%, or less than 5% of the level of nutrient available in a normal and/or healthy environment and/or cell and/or tissue.

In an embodiment, the increase in survival of the one or more NK cell and/or T cell and/or the effect on activity of the one or more NK cell and/or T cell is present in the absence of any known exogenous oxidative stress.

In an embodiment, the oxidative stress comprises the presence of Reactive Oxygen Species (ROS). The oxidative stress may comprise the presence of higher levels of ROS than is present in a normal and/or healthy environment and/or cell and/or tissue.

By “Reactive Oxygen Species” or ROS, we include reactive free radical and non-radical derivatives of oxygen. Free radicals are chemical substances that contain one or more unpaired orbital electrons and are therefore unstable and liable to react with other molecules to form more stable compounds with a lower energy state. In an attempt to achieve this stable state, ROS reacts with proteins, lipids, and DNA within the cell, which can result in damage and even inactivation of cellular components such as enzymes, membranes, and DNA.

In a preferred embodiment the Reactive Oxygen Species (ROS) comprises one or more of hydrogen peroxide (H₂O₂), superoxide anions (O₂⁻), nitric oxide, hydroxyl radicals, hydroxyl ions and/or monocyte-derived ROS; and preferably the Reactive Oxygen Species is hydrogen peroxide (H₂O₂).

By “monocyte-derived ROS” we include ROS that is produced by suppressive monocytes and/or cells in a tumour microenvironment. This may comprise a mixture of different ROS species, for example hydrogen peroxide (H₂O₂), superoxide anions (O₂⁻), nitric oxide, hydroxyl radicals and/or hydroxyl ions.

In an embodiment, the invention provides a use or the method, wherein the NK cell and/or T cell activity is selected from one or more of:
i) anti-cancer or anti-tumour activity;
ii) production and/or release of cytokines;
iii) production and/or release of IFN-γ;
iv) effector function in tumour and/or spheroid tumour structures;
v) specific lysis of a target cell, for example a tumour and/or a cancer cell; and/or
vi) degranulation and/or capacity to degranulate;
vii) ability to regulate or influence other immune cell types, such as Dendritic cells, macrophages or other monocyte/myeloid cell types, or other lymphocyte cell (e.g. NK mediated regulation of T cell activity and vice versa).

By “anti-cancer activity”, we include: a biological activity associated with a decrease in the number of cancer cells, a decrease in metastases, a decrease in cancer cell proliferation, a decrease in cancer cell survival, an increase in killing and/or apoptosis of cancer cells, an increase in life expectancy of a subject having cancer cells, and/or amelioration of a physiological symptom associated with the cancer. “Anti-cancer activity” can also include the prevention of the occurrence of a cancer.

By “anti-tumour activity”, we include a biological activity associated with a decrease in tumour volume, a decrease in the number of tumour cells, a decrease in the number of metastases, decrease in tumour cell proliferation, decrease in tumour cell survival, an increase in killing and/or apoptosis of tumour cells, an increase in life expectancy of a subject having tumour cells, and/or amelioration of a physiological symptoms associated with the cancerous condition. “Anti-tumour activity” can also include the prevention of the occurrence of a tumour and/or tumour cells.

By “degranulation”, we include the release of cytotoxic molecules, such as perforin and granzymes, from secretory vesicles (granules) from the one of T cell and/or NK cell on to the surface of a target cell (e.g. a cancer cell and/or tumour cell).

By “effector function”, we include increased cell death of cancer cells and/or increased tumour elimination and/or increased activation of other immune cells by the one or more NK cell and/or T cell.

In an embodiment, the invention provides a use or a method, wherein the NK cell and/or T cell has an increased resistance to stress-induced cell death, for example oxidative stress-induced cell death, such as Reactive Oxygen Species-induced cell death and/or hydrogen peroxide-induced cell death, preferably where the increased resistance is relative to a NK cell and/or T cell that has not been contacted with and/or treated with and/or exposed to an activator of Nrf2. In a further embodiment, the cell death may be apoptosis. In a further embodiment, the stress may result from treatment of a cancer patient with a therapeutic agent such as chemotherapy and/or radiation.

By “cell death”, we include all types of cell death including apoptosis, necrosis, ferroptosis, mitotic catastrophe, particularly wherein a cell actively pursues a course towards death in response to a certain stimuli or stress (e.g. ROS). Cell death and/or apoptosis may be measured by any suitable method known in the art and/or by using a commercially available kit. For example, cell death and/or apoptosis can be measured using Annexin V and propidium iodide staining followed by flow cytometry in order to determine cell viability. This allows monitoring of all stages, including live, early apoptosis, late apoptosis and necrosis. Methods for determining cell death, apoptosis and/or cell viability, including Annexin V/propidium iodide staining methods, can be found in Cummings and Schnellmann (2004).

In an embodiment, the invention provides a use or method, wherein there is:
(i) an increase of NK cell and/or T cell activity in the presence of the one or more stress;
(ii) a reduction in suppression of NK cell and/or T cell activity caused by the one or more stress; and/or
(iii) an increase in NK cell and/or T cell survival in the presence of the one or more stress;
wherein the increase or reduction is in the range of 1 to 100%, such as at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 40%, 50%, 60%, 70%, 80%, 90%, 100% or greater.

In an embodiment, the invention provides a use or a method, wherein there is:
(i) an increase of NK cell and/or T cell activity in the presence of the one or more stress;
(ii) a reduction in suppression of NK cell and/or T cell activity caused by the one or more stress; and/or
(iii) an increase in NK cell and/or T cell survival in the presence of the one or more stress;
wherein the increase or reduction is in the range of a 1.01-fold to 3-fold change, such as an increase of 1.01-fold, 1.02-fold, 1.03-fold, 1.04-fold, 1.05-fold, 1.06-fold, 1.07-fold, 1.09-fold, 1.1-fold, 1.2-fold, 1.25-fold, 1.3-fold, 1.4-fold, 1.5-fold, 1.6-fold, 1.7-fold, 1.8-fold, 1.9-fold, 2-fold, 2.25-fold, 2.5-fold, 3-fold or greater.

In a preferred embodiment, the increase or reduction is relative to one or more NK cell and/or T cell that has not been contacted with an activator of Nrf2.

The increase or reduction may occur and/or be apparent at 6 hours, 12 hours, 18 hours, 24 hours, 30 hours, 36 hours, 42 hours, 48 hours, 60 hours, 72 hours, 84 hours and/or 96 hours after the activator of Nrf2 is contacted with the one or more NK cell and/or T cell. In particular, the increase or reduction may occur and/or be apparent at 6 hours, 12 hours, 18 hours, 24 hours, 30 hours, 36 hours, 42 hours, 48 hours, 60 hours, 72 hours, 84 hours and/or 96 hours after the contacting step and/or post-contacting step.

The increase or reduction may be short-lived and may return to normal after a period of 24 hours, 30 hours, 36 hours, 42 hours, 48 hours, 60 hours, 72 hours, 84 hours and/or 96 hours. By “normal” we include the level of activity for one or more NK cell and/or T cell that has not been contacted with an activator of Nrf2.

The one or more NK cell and or T cell may be obtained from a subject by any suitable method for obtaining lymphocytes from a subject's blood, a subject's tumour sample, and/or another tissue from a subject. The one of more T cell and/or NK cell may be obtained from a unit of blood collected from a subject using any suitable techniques known in the art such as Ficoll™ separation. The one or more NK cell and/or T cell may be obtained using an Elutra™ cell separation system (TerumoBCT Inc., USA).

In an embodiment of the second aspect of the invention, the method comprises a step of: obtaining one or more NK cell and/or T cell by apheresis (for example leukapheresis); and/or by taking a blood sample (e.g. a peripheral blood sample or an umbilical cord blood sample); and/or by taking a sample from an ascites; and/or by draining one or more lymph node; and/or by biopsy (for example by biopsy of a tumour, optionally by biopsy of a solid tumour) and/or by venesection.

In an embodiment, cells from the circulating blood of a subject are obtained by apheresis or any of the aforementioned methods. The product typically contains lymphocytes, including T cells, monocytes, granulocytes, NK cells, B cells, other nucleated white blood cells, red blood cells, and platelets. It will be appreciated that the cells may be washed to remove the plasma fraction and to place the cells in an appropriate buffer or media for subsequent processing steps. For example, the cells may be washed with phosphate buffered saline (PBS). Alternatively, the wash solution lacks calcium and may lack magnesium or may lack many if not all divalent cations. Initial activation steps in the absence of calcium can lead to magnified activation. A washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, for example, Ca-free, Mg-free PBS, PlasmaLyte A, or other saline solution with or without buffer. Alternatively, the undesirable components of the sample may be removed, and the cells directly resuspended in culture media.

The one or more T cell and/or NK cell may be isolated from peripheral blood lymphocytes by lysing the red blood cells and depleting the monocytes, for example, by centrifugation through a PERCOLL™ (Sigma-Aldrich) gradient or by counter-flow centrifugal elutriation. Specific subpopulations of T cells, such as CD3+, CD28+, CD4+, CD8+, CD45RA+, and CD45RO+ T cells, or NK cells, such as CD3−CD56+ may be further isolated by positive or negative selection techniques known in the art. For example, T cells may be isolated by incubation with anti-CD3/anti-CD28 (e.g. 3×28)-conjugated beads, such as DYNABEADS® M-450 CD3/CD28 T (Thermofisher), for a time period sufficient for activation of the T cell. Additionally or alternatively, a population of T cells and/or NK cells may be enriched by negative selection, for instance by a combination of antibodies directed to surface markers unique to the negatively selected cells. Cell sorting and/or selection via negative magnetic immunoadherence or flow cytometry may be used.

If desired, the one or more NK cell and/or T cell, purified by the above methods, may be genetically manipulated by transfection with viral vectors and/or by CRISPR technology and/or by any other genetic technology in to express relevant cell receptors, such as T cell receptors and/or Chimeric Antigen Receptors.

In an embodiment of the second aspect of the invention, the method further comprises a step of administering the one or more NK cell and/or T cell to a patient in need thereof.

By “a patient in need thereof”, we include a patient who may benefit from the administration of the one or more NK cell and/or T cell. For instance, the patient may be suffering from a condition or disease (e.g. cancer or a viral infection) that can be alleviated, treated and/or improved by administration of the one or more NK cell and/or

T cell. The patient may be likely to suffer from a condition or disease (e.g. cancer or a viral infection) and the one or more NK cell and/or T cell may be administered as a preventative measure to prevent onset of said condition or disease.

NK cells and/or T cells generated by the methods and uses of the invention will be beneficial in the treatment and/or prevention of viral infections.

NK cells and/or T cells generated by the methods and uses of the invention will be beneficial in the treatment and/or prevention of all cancers, but will be particularly effective in solid tumours in which ROS are present at increased levels and/or are believed to suppress such cell therapies.

In a preferred embodiment, the patient in need thereof has cancer, optionally wherein the cancer is characterised by the presence of a stress, such as oxidative stress, hypoxia, reoxygenation, starvation.

By “cancer”, we include a disease characterised by the rapid and uncontrolled growth of aberrant cells. Cancer cells can spread locally or through the bloodstream and lymphatic system to other parts of the body. Examples of various cancers include bladder cancer, bone cancer, breast cancer, colon cancer, cervical cancer, rectal cancer, endometrial cancer, oesophageal cancer, ovarian cancer, gastric cancer, kidney cancer, leukaemia, liver cancer, lung cancer, skin cancer including basal cell carcinoma, squamous cell carcinoma and melanoma, also including non-skin located uveal melanoma and mucosal melanoma, lymphoma, pancreatic cancer, prostate cancer, testicular cancer, thyroid cancer, and the like.

Cancers that may be treated include tumours that are not vascularised, or not yet substantially vascularized, as well as vascularised tumours. The cancers may include non-solid tumours (such as haematological tumours, for example, leukaemia and lymphomas) or may include solid tumours. Types of cancers to be treated with the one or more NK cell and/or T cell include, but are not limited to, carcinoma, blastoma, and sarcoma, and leukaemia or lymphoid malignancies, benign and malignant tumours, and malignancies, e.g., sarcomas, carcinomas, and melanomas. Adult tumours/cancers and paediatric tumours/cancers are included. The cancer can be melanoma. The cancer can be lung cancer. The cancer can be head and neck cancer.

The cancer can be cervical cancer. The cancer can be ovarian cancer. The cancer can be colorectal cancer.

Hematologic cancers are cancers of the blood or bone marrow. Examples of haematological (or hematogenous) cancers include leukaemia, including acute leukaemia (such as acute lymphocytic leukaemia, acute myelocytic leukaemia, acute myelogenous leukaemia and myeloblastic, promyelocytic, myelomonocytic, monocytic and erythroleukemia), chronic leukaemia (such as chronic myelocytic (granulocytic) leukaemia, chronic myelogenous leukaemia, and chronic lymphocytic leukaemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukaemia and myelodysplasia.

Solid tumours are abnormal masses of tissue that usually do not contain cysts or liquid areas. Solid tumours can be benign or malignant. Different types of solid tumours are named for the type of cells that form them (such as sarcomas, carcinomas, and lymphomas). Examples of solid tumours, such as sarcomas and carcinomas, include fibrosarcoma, myosarcoma, liposarcoma, chondrosarcoma, osteosarcoma, synovioma, mesothelioma, Ewing's tumour, leiomyosarcoma, rl rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumour, cervical cancer, testicular tumour, seminoma, bladder carcinoma, melanoma, and CNS tumours (such as a glioma (such as brainstem glioma and mixed gliomas), glioblastoma (also known as glioblastoma multiforme), astrocytoma, CNS lymphoma, germinoma, medulloblastoma, Schwannoma craniopharyngioma, ependymoma, pinealoma, hemangioblastoma, acoustic neuroma, oligodendroglioma, meningioma, neuroblastoma, retinoblastoma, and brain metastases).

In a preferred embodiment, the patient in need thereof has previously been treated for cancer by an alternative therapeutic agent and/or method, and the risk of recurrence or progression of the cancer is reduced by administering the one or more NK cell and/or T cell to a patient.

By “alternative therapeutic agent”, we include all anti-cancer agents or anti-angiogenesis agents aside from those of the present invention. It will be appreciated that the therapeutic agent may be one that is known to be suitable for combating the disease towards which the treatments of the invention are directed.

The alternative therapeutic agent and/or anti-cancer agent may be an anti-cancer antibody, an anti-CD20 agent (e.g. Ofatumumab or Rituximab), a chimeric antigen receptor T cell (e.g. a CD19+ chimeric antigen receptor T cell) and/or non-activated monocytes.

The anti-cancer agent may be selected from alkylating agents including nitrogen mustards such as mechlorethamine (HN2), cyclophosphamide, ifosfamide, melphalan (L-sarcolysin) and chlorambucil; ethylenimines and methylmelamines such as hexamethylmelamine, thiotepa; alkyl sulphonates such as busulphan; nitrosoureas such as carmustine (BCNU), lomustine (CCNU), semustine (methyl-CCNU) and streptozocin (streptozotocin); and triazenes such as decarbazine (DTIC; dimethyltriazenoimidazole-carboxamide); antimetabolites including folic acid analogues such as methotrexate (amethopterin); pyrimidine analogues such as fluorouracil (5-fluorouracil; 5-FU), floxuridine (fluorodeoxyuridine; FUdR) and cytarabine (cytosine arabinoside); and purine analogues and related inhibitors such as mercaptopurine (6-mercaptopurine; 6-MP), thioguanine (6-thioguanine; TG) and pentostatin (2′-deoxycoformycin); natural products including vinca alkaloids such as vinblastine (VLB) and vincristine; epipodophyllotoxins such as etoposide and teniposide; antibiotics such as dactinomycin (actinomycin D), daunorubicin (daunomycin; rubidomycin), doxorubicin, bleomycin, plicamycin (mithramycin) and mitomycin (mitomycin C); enzymes such as L-asparaginase; and biological response modifiers such as interferon alphenomes; miscellaneous agents including platinum coordination complexes such as cisplatin (cis-DDP) and carboplatin; anthracenedione such as mitoxantrone and anthracycline; substituted urea such as hydroxyurea; methyl hydrazine derivative such as procarbazine (N-methylhydrazine, MIH); and adrenocortical suppressant such as mitotane (o,p′-DDD) and aminoglutethimide; taxol and analogues/derivatives; cell cycle inhibitors; proteosome inhibitors such as Bortezomib (Velcade®); signal transductase (e.g. tyrosine kinase) inhibitors such as Imatinib (Glivec®), COX-2 inhibitors, and hormone agonists/antagonists such as flutamide and tamoxifen.

The anti-cancer agent may work by any of the following mechanisms of action: Alkylating agent, Topoisomerase I inhibitor, Topoisomerase II inhibitor, RNA/DNA antimetabolite, DNA antimetabolite and Antimitotic agent. The US NIH/National Cancer Institute website lists 122 compounds (http://dtp.nci.nih.gov/docs/cancer/searches/standard_mechanism.html), all of which may be used an an anti-cancer agent. They include Alkylating agents including Asaley, AZQ, BCNU, Busulfan, carboxyphthalatoplatinum, CBDCA, CCNU, CHIP, chlorambucil, chlorozotocin, cis-platinum, clomesone, cyanomorpholino-doxorubicin, cyclodisone, dianhydrogalactitol, fluorodopan, hepsulfam, hycanthone, melphalan, methyl CCNU, mitomycin C, mitozolamide, nitrogen mustard, PCNU, piperazine, piperazinedione, pipobroman, porfiromycin, spirohydantoin mustard, teroxirone, tetraplatin, picoplatin (SP-4-3) (cis-aminedichloro(2-methylpyridine)Pt(II)), thio-tepa, triethylenemelamine, uracil nitrogen mustard, Yoshi-864; anitmitotic agents including allocolchicine, Halichondrin B, colchicine, colchicine derivative, dolastatin 10, maytansine, rhizoxin, taxol, taxol derivative, thiocolchicine, trityl cysteine, vinblastine sulphate, vincristine sulphate; Topoisomerase I Inhibitors including camptothecin, camptothecin, Na salt, aminocamptothecin, 20 camptothecin derivatives, morpholinodoxorubicin; Topoisomerase II Inhibitors including doxorubicin, amonafide, m-AMSA, anthrapyrazole derivative, pyrazoloacridine, bisantrene HCL, daunorubicin, deoxydoxorubicin, mitoxantrone, menogaril, N,N-dibenzyl daunomycin, oxanthrazole, rubidazone, VM-26, VP-16; RNA/DNA antimetabolites including L-alanosine, 5-azacytidine, 5-fluorouracil, acivicin, 3 aminopterin derivatives, an antifol, Baker's soluble antifol, dichlorallyl lawsone, brequinar, ftorafur (pro-drug), 5,6-dihydro-5-azacytidine, methotrexate, methotrexate derivative, N-(phosphonoacetyl)-L-aspartate (PALA), pyrazofurin, trimetrexate; DNA antimetabolites including, 3-HP, 2′-deoxy-5-fluorouridine, 5-HP, alpha-TGDR, aphidicolin glycinate, ara-C, 5-aza-2′-deoxycytidine, beta-TGDR, cyclocytidine, guanazole, hydroxyurea, inosine glycodialdehyde, macbecin II, pyrazoloimidazole, thioguanine and thiopurine.

The anti-cancer agent may be selected from cisplatin; carboplatin; picoplatin; 5-flurouracil; paclitaxel; mitomycin C; doxorubicin; gemcitabine; tomudex; pemetrexed; methotrexate; irinotecan, fluorouracil and leucovorin; oxaliplatin, 5-fluorouracil and leucovorin.

In an embodiment of the method of the second aspect of the invention, the step of contacting the NK cell and/or T cell with the activator of Nrf2 occurs ex vivo and/or in vitro.

By “ex vivo”, we include that the one or more NK cell and/or T cell is contacted with the activator of Nrf2 outside of a body (e.g. outside of a human or animal body), preferably after having been removed from such a body and/or before being administered to such a body.

By “in vitro”, we include that the one or more NK cell and/or T cell is contacted with the activator of Nrf2 within laboratory equipment and/or in a controlled environment. For example, in vitro contacting might be carried out in a test tube, flask, cell expansion bag, bioreactor or petri dish.

In a third aspect, the invention provides an activator of Nrf2 for use in treating cancer in a patient, wherein the cancer is characterised by the presence of one or more stress, and wherein the treatment comprises the step of contacting the activator of Nrf2 with one or more NK cell and/or T cell.

In a fourth aspect, the invention provides the use of an activator of Nrf2 in the manufacture of a medicament for treating cancer in a patient, wherein the cancer is characterised by the presence of one or more stress, and wherein the treatment comprises the step of contacting the activator of Nrf2 with one or more NK cell and/or T cell.

In a fifth aspect, the invention provides a method for treating cancer in a patient, wherein the cancer is characterised by the presence of one or more stress, and wherein the method comprises the step of contacting one or more NK cell and/or T cell with an activator of Nrf2.

The activities, stresses, activators of Nrf2, or any other feature in respect of the third, fourth and fifth aspects of the invention may be as defined for any of the previous aspects.

In an embodiment of the invention, the activator of Nrf2 prevents and/or reduces the suppression of NK cell and/or T cell activity caused by one or more stress; and/or induces and/or maintains and/or increases NK and/or T cell activity in the presence of one or more stress; and/or increases NK cell and/or T cell survival in response to one or more stress. The one or more stress may be in a tumour microenvironment.

In an embodiment of the invention, the one or more stress is oxidative stress, hypoxia, reoxygenation and/or starvation; preferably the stress is oxidative stress.

In an embodiment of the invention, the one or more NK cell and/or T cell activity is selected from one or more of:
i) anti-cancer cell or anti-tumour cell activity;
ii) production and/or release of cytokines;
iii) production and/or release of IFN-γ;
iv)) effector function in tumour and/or spheroid tumour structures;
v) specific lysis of a target cell, for example a tumour and/or a cancer cell;
vi) degranulation and/or capacity to degranulate; and/or
vii) ability to regulate and/or influence other immune cell types, such as Dendritic cells, macrophages or other monocyte/myeloid cell types, or other lymphocyte cell (e.g. NK mediated regulation of T cell activity and vice versa).

In an embodiment of the invention, the one or more NK cell and/or T cell has an increased resistance to stress-induced cell death, for example reactive oxygen species-induced cell death, such as hydrogen peroxide-induced cell death; preferably the increased resistance is relative to a NK cell and/or T cell that has not been contacted with and/or treated with and/or exposed to an activator of Nrf2. Preferable the cell death is apoptosis.

In an embodiment of the invention, the activator of Nrf2 is auranofin (triethylphosphine gold), dimethyl fumarate (DMF), sulforaphane, curcumin, resveratrol, naringenin and/or agmatine. In alternative embodiments, the activator of Nrf2 is one or more activator selected from the group consisting of: caffeic acid phenethyl ester, methylglyoxal, itaconate, tertiary butylhydroquinone (tBHQ), Zn²⁺, 15-deoxy-412,14-prostaglandin J2, 1,2-naphthoquinone, cadmium chloride, methyl-mercury, arsenic, diethylmaleate, dexamethasone (DEX), Bardoxolone-methyl (CDDO-Me), RTA-408 (omaveloxolone), ALKS-8700, Oltiprazm Ursodiol, Sulforadex (SFX-01), ITH12674, CXA-10 and/or any of the peptide inhibitors listed in Table 2 of Robledinos-Antón et al. 2019 (Wu and Papagiannakopoulos, 2020; Robledinos-Antón et al. 2019), cinnamic acid ester, cinnamic aldehyde, flavonols (e.g. Myricetin, Quercetin), flavan-3-ols (e.g. Catechin, Epicatechin, Epicatechin gallate, Epigallocatechin gallate), flavanonols (e.g. Taxifolin), hydroxyalkenals (e.g. 4-Hydroxynonenal), prostaglandins (e.g. 15d-PGJ2z, Prostaglandin A1), quinones (e.g. Juglone, Naphthazarin, PQQ Cofactor), polyphenols (e.g. Ellagic acid); benzene derivatives (e.g. Dinitrochlorobenzene (CDNB, DNCB), Dinitrofluorobenzene (FDNB, DNFB)), isothiocyanates (e.g. Sulforaphane, Benzyl isothiocyanate (BITC), phenethyl isothiocyanate (PEITC), allyl isothiocyanate (AITC)), arsenic compounds (e.g. Arsenite (AsIII), Monomethylarsonous acid, Arsenate (AsV), Arsenic trioxide, Phenylarsine oxide), gold compounds (e.g. Auranofin, Aurothiomalate, Gold (III) chloride), platinum compounds (e.g. Cisplatin, Nedaplatin, Oxaliplatin), gadolinium compound (e.g. Motexafin gadolinium); mercury compounds (e.g. Mercuric chloride, Monomethylmercury), sulphur mustard (2-chloroethyl ethyl sulfide);

nitrosourea (e.g. Carmustine (BCNU)), acetaminophen metabolite (e.g. NAPQI), Schistosomicide (e.g. Oltipraz), statins (e.g. Simvastatin, Fluvastatin), unsaturated aldehyde (e.g. Acrolein), nitrogen mustard (e.g. Chlorambucil, Melphalan), and/or Dimethyl (E)-butenedioate. The activator or Nrf2 may be a non-conding RNA, such as a mircroRNA, that allows for RNA silencing of Keap1 and/or TrxR1.

In an embodiment of the invention, the oxidative stress comprises the presence of Reactive Oxygen Species, optionally wherein the Reactive Oxygen Species, comprises one or more of hydrogen peroxide (H₂O₂), superoxide anions (O₂) nitric oxide, hydroxyl radicals, hydroxyl ions and/or monocyte-derived ROS; preferably wherein the Reactive Oxygen Species is hydrogen peroxide (H₂O₂).

In an embodiment of the invention, the one or more NK cell and/or T cell is contacted with the activator of Nrf2 ex vivo and/or in vitro.

In an embodiment of any aspect of the invention, the one or more NK cell and/or T cell is autologous. Thus, the T-cell can be autologous. In an embodiment of any aspect of the invention, the one or more NK cell and/or T cell is obtained from the patient. The cell can be obtained from a patient in a sample, such as a biopsy. The T-cell can be a tumor infiltrating lymphocyte (TIL).

By “autologous”, we include that the one or more NK cell and/or T cell is derived from a patient and is subsequently re-introduced into the same patient.

One aspect of the present invention relates to a method for promoting regression of a cancer in a mammal by expanding tumor infiltrating lymphocytes (TILs) into a therapeutic population of TILs comprising:
(a) culturing autologous T cells by obtaining a first population of TILs from a tumor resected from a mammal,
(b) performing a first expansion by culturing the first population of TILs in a cell culture medium comprising IL-2 and one or more TME stimulators to produce a second population of TILS;
(c) performing a second expansion by supplementing the cell culture medium of the second population of TILs with additional IL-2, anti-CD3 antibodies, and antigen presenting cells (APCs), to produce a third population of TILs, wherein the third population of TILs is a therapeutic population;
(d) contacting the third population of TILs with an activator of Nrf2, and
(e) after administering nonmyeloablative lymphodepleting chemotherapy, administering to the mammal the therapeutic population of T cells, wherein the T cells administered to the mammal, whereupon the regression of the cancer in the mammal is promoted.

A further aspect of the present invention relates to a method for treating a subject with cancer comprising administering expanded tumor infiltrating lymphocytes (TILs) comprising:
(a) culturing autologous T cells by obtaining a first population of TILs from a tumor resected from a mammal,
(b) performing a first expansion by culturing the first population of TILs in a cell culture medium comprising IL-2 and one or more TME stimulators to produce a second population of TILS;
(c) performing a second expansion by supplementing the cell culture medium of the second population of TILs with additional IL-2, anti-CD3 antibodies, and antigen presenting cells (APCs), to produce a third population of TILs, wherein the third population of TILs is a therapeutic population;
(d) contacting the third population of TILs with an activator of Nrf2, and (e) after administering nonmyeloablative lymphodepleting chemotherapy, administering to the mammal the therapeutic population of T cells, wherein the T cells administered to the mammal, whereupon the regression of the cancer in the mammal is promoted.

Another aspect of the present invention relates to a method for expanding tumor infiltrating lymphocytes (TILs) into a therapeutic population of TILs comprising:
(a) culturing autologous T cells by obtaining a first population of TILs from a tumor resected from a mammal,
(b) performing a first expansion by culturing the first population of TILs in a cell culture medium comprising IL-2 and one or more TME stimulators to produce a second population of TILS;
(c) performing a second expansion by supplementing the cell culture medium of the second population of TILs with additional IL-2, anti-CD3 antibodies, and antigen presenting cells (APCs), to produce a third population of TILs, wherein the third population of TILs is a therapeutic population, and
(d) contacting the third population of TILs with an activator of Nrf2.

In one or more embodiments, the therapeutic population of T cells is used to treat a breast cancer. In one or more embodiments, the therapeutic population of T cells is used to treat renal cell cancer. In one or more embodiments, the therapeutic population of T cells is used to treat bladder cancer. In one or more embodiments, the therapeutic population of T cells is used to treat melanoma. In one or more embodiments, the therapeutic population of T cells is used to treat cervical cancer. In one or more embodiments, the therapeutic population of T cells is used to treat gastric cancer. In one or more embodiments, the therapeutic population of T cells is used to treat colorectal cancer. In one or more embodiments, the therapeutic population of T cells is used to treat lung cancer. In one or more embodiments, the therapeutic population of T cells is used to treat head and neck cancer. In one or more embodiments, the therapeutic population of T cells is used to treat ovarian cancer. In one or more embodiments, the therapeutic population of T cells is used to treat Hodgkin lymphoma. In one or more embodiments, the therapeutic population of T cells is used to treat pancreatic cancer. In one or more embodiments, the therapeutic population of T cells is used to treat liver cancer. In one or more embodiments, the therapeutic population of T cells is used to treat sarcomas.

In one or more embodiments, steps (a) through (c) or (d) are performed within a period of about 20 days to about 45 days. In one or more embodiments, steps (a) through (c) or (d) are performed within a period of about 20 days to about 40 days. In one or more embodiments, steps (a) through (c) or (d) are performed within a period of about 25 days to about 40 days. In one or more embodiments, steps (a) through (c) or (d) are performed within a period of about 30 days to about 40 days. In one or more embodiments, steps (a) through (b) are performed within a period of about 10 days to about 28 days. In one or more embodiments, steps (a) through (b) are performed within a period of about 10 days to about 20 days.

In one or more embodiments, step (c) is performed within a period of about 12 days to about 18 days. In one or more embodiments, step (c) is performed within a period of about 10 days to about 28 days. In one or more embodiments, step (c) is performed within a period of about 10 days to about 20 days. In one or more embodiments, step (c) is performed within a period of about 12 days to about 18 days.

In one or more embodiments, step (b) results in 1×10⁶ to 1×10⁷ cells, such as 2×10⁶ to 5×10⁶ cells. In one or more embodiments, step (b) results in 5×10⁶ to 1×10⁷ cells. In one or more embodiments, step (b) results in 1×10⁶ to 5×10⁷ cells. In one or more embodiments, step (b) results in 1×10⁷ to 5×10⁷ cells. In one or more embodiments, step (c) results in 1×10⁷ to 1×10¹² cells, such as 1×10⁸ to 5×10⁹ cells, such as 1×10⁹ to 5×10⁹ cells, such as 1×10⁸ to 5×10¹⁰ cells, such as 1×10⁹ to 5×10¹¹ cells. In one or more embodiments, step (c) results in 1×10⁷ to 1×10¹⁰ cells. In one or more embodiments, step (c) results in 1×10⁷ to 1×10⁹ cells. In one or more embodiments, step (c) results in 1×10⁷ to 1×10⁸ cells.

In one or more embodiments, the APCs are artificial APCs (aAPCs) or allogeneic feeder cells.

In one or more embodiments, the therapeutic population of TILs are infused into a patient.

In one or more embodiments, the cells are removed from the cell culture and cryopreserved in a storage medium prior to performing step (c).

In one or more embodiments, the method further comprises the step of transducing the first population of TILs with an expression vector comprising a nucleic acid encoding a chimeric antigen receptor (CAR) comprising a single chain variable fragment antibody fused with at least one endodomain of a T-cell signaling molecule.

In one or more embodiments, step (c) further comprises a step of removing the cells from the cell culture medium.

In one or more embodiments, step (a) further comprises processing of the resected tumor into multiple tumor fragments, such as 4 to 50 fragments, such as 20 to 30 fragments.

In one or more embodiments, the fragments have a size of about 5 to 50 mm³, In one or more embodiments, the fragments have a size of about 50 mm³. In one or more embodiments, the fragments have a size of about 0.1 to 10 mm³. In one or more embodiments, the fragments have a size of about 0.1 to 1 mm³. In one or more embodiments, the fragments have a size of about 0.5 to 5 mm³. In one or more embodiments, the fragments have a size of about 1 to 10 mm³. In one or more embodiments, the fragments have a size of about 1 to 3 mm³.

The cells can be from a mammal. In one or more embodiments, the mammal is a human.

In one or more embodiments, the cell culture medium is provided in a container selected from the group consisting of a G-Rex container (Wilson Wolf Manufacturing, US) and a Xuri cellbag (Cytiva, US).

An aspect relates to a population of tumor infiltrating lymphocytes (TILs) obtainable by any of the methods of the present invention defined herein.

A further aspect relates to expanded tumor infiltrating lymphocytes (TILs) for use in treating a subject with cancer, the treatment comprising the steps of: culturing autologous T cells by obtaining a first population of TILs from a tumor resected from a mammal performing a first expansion by culturing the first population of TILs in a cell culture medium comprising IL-2 and one or more TME stimulators to produce a second population of TILs; performing a second expansion by supplementing the cell culture medium of the second population of TILs with additional IL-2, anti-CD3, and antigen presenting cells (APCs), to produce a third population of TILs, wherein the third population of TILs is a therapeutic population; contacting the third population of TILs with an activator of Nrf2, and after administering nonmyeloablative lymphodepleting chemotherapy, administering to the mammal the therapeutic population of T cells, wherein the T cells administered to the mammal, whereupon the regression of the cancer in the mammal is promoted.

A further aspect relates to a population of tumor infiltrating lymphocytes (TILs) obtainable by a method comprising: culturing autologous T cells by obtaining a first population of TILs from a tumor resected from a mammal performing a first expansion by culturing the first population of TILs in a cell culture medium comprising IL-2 and one or more TME stimulators to produce a second population of TILs; and performing a second expansion by supplementing the cell culture medium of the second population of TILs with additional IL-2, anti-CD3, and antigen presenting cells (APCs), to produce a third population of TILs, wherein the third population of TILs is a therapeutic population, and contacting the third population of TILs with an activator of Nrf2.

In an embodiment of any aspect of the invention, the one or more NK cell and/or T cell is allogenic. In an embodiment of any aspect of the invention, the one or more NK cell and/or T cell is obtained from a donor.

By “allogenic”, we include that the one or more NK cell and/or T cell is derived from a donor and/or a person that is not the patient. The donor may be a related or unrelated or recipient subject.

In an embodiment of the invention, the treatment or method comprises a step of obtaining one or more NK cell and/or T cell from the patient or from a donor. In a preferred embodiment of the invention, the one or more NK cell and/or T cell is obtained: by apheresis (for example leukapheresis); by taking a blood sample (e.g. a peripheral blood sample or an umbilical cord blood sample); by taking a sample from an ascites; by draining one or more lymph node; and/or by a biopsy (for example by a biopsy of a tumour, optionally by a biopsy of a solid tumour) and/or by venesection.

Activators of Nrf2, for example auranofin, have previously been have shown to be toxic to cells in general, including lymphocytes. There have been various studies showing the use of auranofin in cancer biology to directly kill cancer and/or tumour cells (Roder and Thomson 2015). Furthermore, auranofin has been shown to be toxic to lymphocytes infected with HIV (Chirullo et al. 2013). Therefore it is surprising and unexpected that the present inventors have shown that these compounds can be used to give beneficial and protective effects in lymphocytes such as T cells and NK cells, even when incubated with the cells for long period of time (such as 18 hours, 24 hours or longer).

In an embodiment of the invention, the one or more NK cell and/or T cell is contacted with an activator of Nrf2 for up to 48 hours, optionally between 0.5 and 24 hours. Preferably, the one or more NK cell and/or T cell is contacted with an activator of Nrf2 for 0.1 hours, 0.2 hours, 0.3 hours, 0.4 hours, 0.5 hours, 0.75 hours, 1 hour, 2 hours, 3 hours, 4 hours, 5 hours, 6 hours, 7 hours, 8 hours, 9 hours, 10 hours, 11 hours, 12 hours, 14 hours, 16 hours, or 18 hours or 24 hours.

The one of more NK cell and/or T cell may be incubated with the activator of Nrf2 activator for a given period of time (a “contacting step”) before the activator of Nrf2 is subsequently removed e.g. by washing the one or more NK cell and/or T cell. Optionally, the one or more NK cell and/or T cell may then undergo a “post-contacting step”, whereby the one or more NK cell and/or T cell may be incubated in the absence of the activator of Nrf2 prior to being administered to the patient and/or being utilised in the treatment of cancer in the patient. The post-contacting step is particularly beneficial in situations where a short contacting step is employed (e.g. the activator of Nrf2 is pulsed with the one or more NK cell and/or T cell and removed). When shorter contacting step is utilised (e.g. 30 minutes), the post-contacting step may ensure that there is sufficient time for the effect of the activator of Nrf2 to induce the beneficial effects on the one or more NK cell and/or T cell (e.g. by effecting transcription of relevant detoxifying genes and/or effector target genes in the one or more NK cell and/or T cell) before they are administered to a patient and/or are utilised in the treatment of cancer.

Surprisingly, the inventors have found that there is no significant difference in the effects on the capacity to resist stress (e.g. ROS) by NK and/or T cells that are pulsed with an activator of Nrf2 for a short duration (e.g. 30 minutes) followed by a post-contacting step (e.g. 17.5 hours) compared to cells that are incubated with the activator of Nrf2 for a longer contacting step of the same total duration (e.g. an 18 hour contacting step).

Based on the teaching herein, it would be within the remit of the skilled person to select an appropriate duration for the contacting step and/or post-contacting step, which may depend on factors such as the specific type of cells utilised.

In an embodiment, the invention further comprises an optional post-contacting step, wherein the one or more NK cell and/or T cell are incubated in the absence of the activator of Nrf2; optionally wherein the activator of Nrf2 is washed away from the one or more NK cell and/or T cell.

By “post-contacting step”, we include a period of time where the one or more NK cell and/or T cell is incubated in the absence of the activator of Nrf2. The activator of Nrf2 may be removed by any suitable method, including washing away the activator of Nrf2 from the one or more NK cell and/or T cell.

Preferably, the post-contacting step occurs in vitro. The post-contacting step may occur in vivo and/or after a step of administration of the one or more NK cell and/or T cell to the patient.

In a preferred embodiment, the post-contacting step is performed for up to 48 hours, optionally for between 0.5 hours to 24 hours. Preferably the post-contact step is performed for 0.1 hours, 0.2 hours, 0.3 hours, 0.4 hours, 0.5 hours, 0.75 hours, 1 hour, 2 hours, 3 hours, 4 hours, 5 hours, 6 hours, 7 hours, 8 hours, 9 hours, 10 hours, 11 hours, 12 hours, 14 hours, 16 hours 17.5 hours, or 18 hours or 24 hours.

In an embodiment of the invention, the total duration of the contacting step and/or post-contacting step is at least 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 20 hours, 22 hours, 24 hours, 48 hours, 72 hours or greater. In a preferred embodiment, the total duration of the contacting step and/or post-contacting step is between 4 and 24 hours, and preferably between 6 and 18 hours.

In a preferred embodiment, the one or more NK cell and/or T cell is one or more NK cell and the total duration of the contacting step and/or post-contacting step is at least 4 hours and is preferably between 8 and 24 hours.

In a preferred embodiment, the one or more NK cell and/or T cell is one or more T cell and the total duration of the contacting step and/or post-contacting step is at least 6 hours and/or is preferably between 8 hours and 24 hours.

In an embodiment, the method or treatment of the invention further comprises a step of administering the cells to the patient, optionally after the contacting step and/or a post-contacting step.

The dosage of the one or more NK cell and/or T cell to be administered to a patient will vary with the precise nature of the condition being treated and the recipient of the treatment. The scaling of dosages for human administration can be performed according to art-accepted practices by a physician depending on various factors. Preferably, the method or treatment involved administering a therapeutically effective dose of the one or more NK cell and/or T cell to the patient.

The term “therapeutically effective dose” refers to the amount of the one or more NK cell and/or T cell that will elicit the biological or medical response of a tissue, system, or patient that is being sought by the researcher, veterinarian, medical doctor or another clinician. The term “therapeutically effective dose” includes the amount of NK cells and/or T cells that, when administered, is sufficient to prevent the development of, or alleviate to some extent, one or more of the signs or symptoms of the disorder or disease being treated (e.g. cancer). The therapeutically effective dose will vary depending on the type of NK cell and/or T cell, the disease (e.g. the type of cancer) and its severity and the age, weight, etc., of the patient to be treated

Wherein the one or more NK cell and/or T cell is a tumour-infiltrating lymphocyte (TIL), a dose of between 1 to 100 billion cells may be administered to the patient. Wherein the one or more NK cell and/or T cell is a chimeric antigen receptor (CAR) T cell, a dose of between 2×10⁵/kg and 2×10⁷/kg may be administered to the patient. Wherein the one or more NK cell and/or T cell is a NK cell, a dose of between 150×10⁶/kg and 500×10⁶/kg may be administered to a patient.

The one or more NK cell and/or T cells may be administered to the patient in a composition comprising a pharmaceutically acceptable diluent, excipient or carrier. Carriers suitable for administration of the compounds provided herein include any such carriers known to those skilled in the art to be suitable for the particular mode of administration. Carriers may include, for example, water, saline, aqueous dextrose, glycerol, glycols, ethanol, and the like, to thereby form a solution or suspension. If desired, the composition to be administered can also contain minor amounts of nontoxic auxiliary substances such as wetting agents, emulsifying agents, solubilizing agents, pH buffering agents and the like, for example, acetate, sodium citrate, cyclodextrine derivatives, sorbitan monolaurate, triethanolamine sodium acetate, triethanolamine oleate, and other such agents.

Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ (both Sigma-Aldrich) or polyethylene glycol (PEG).

If administered intravenously, suitable carriers may include physiological saline or phosphate buffered saline (PBS), and solutions containing thickening and solubilizing agents, such as glucose, polyethylene glycol, and polypropylene glycol and mixtures thereof. These can also be Plasmalyte A (Baxter, US), DMSO, CS10, Human serum albumin (“HSA”), or Pentaisomaltose.

It will be appreciated by the skilled person that the administration of the one or more NK cell and/or T cell of the invention may be by any suitable method that is known in art. In a preferred embodiment of the invention, the one or more NK cell and/or T cell is administered by infusion and/or injection and/or intravenously. The cells can undergo cryopreservation before infusion and/or injection and/or intravenous administration.

In an embodiment of the invention, the activator of Nrf2 is removed and/or washed away before an effective amount of the one or more NK cell and/or T cell is administered to the patient. For example, the one or more NK cell and/or T cell may be washed in a buffer, such as Plasmalyte (plus HSA), or growth media (e.g. RPMI) prior to administration.

In an embodiment of the invention, the effect on the one or more NK cell and/or T cell activity and/or survival is present for sufficient time to allow for the treatment and/or prevention of cancer.

The skilled person would understand that a sufficient time is a duration that is long enough to have a therapeutic effect which treats and/or prevents a cancer. This may be a duration that is long enough for an anti-cancer activity and/or anti-tumour activity to occur.

In a preferred embodiment of the invention, wherein the effect on NK cell and/or T cell activity and/or survival is present for at least 24 hours, at least 30 hours, at least 36 hours, at least 42 hours, at least 48 hours, at least 54 hours, at least 60 hours, at least 66 hours, and/or at least 72 hours after the contacting step and/or the post-contacting step and/or after the activator of Nrf2 is removed and/or washed away from the one or more NK cell and/or T cell.

In a preferred embodiment of the invention, the effect on NK cell and/or T cell activity and/or survival is reversible and/or is no longer present at least 72 hours, at least 84 hours or at least 96 hours after the contacting step and/or the post-contacting step and/or after the activator of Nrf2 is removed and/or washed away from the one or more NK cell and/or T cell.

Additionally, the inventors have surprisingly found that the beneficial effects on NK cell and/or T cell activity caused by the activator of Nrf2 are relatively long-lasting (e.g. at least 72 hours) after the contacting and/or post-contacting. The effects are sufficiently long-lived to be applicable to a cell therapy context. This finding is surprising because treatments that modify NK cell and/or T cell activity are usually short-lived. For example, cytokine-mediated changes to NK cell and/or T cell activity is normally reversed within a much shorter time frame (typically within 24 hours). RNA silencing methods also provide short-lived effects. For example, NK cells treated with IL-15 or IL-2 have reduced killing capacity for just 24 hours after cytokine withdrawal, and the cytokine withdrawal increased NK cell death (apoptosis) within 24 hours (Mao et al. 2016). Similarly, Larsen et al. treated expanded T cells with a cytokine for 14 days; after withdrawal of the cytokine, T cells populations start to die, and by 48 hours the majority of the effector memory cells (EmE) were dead (FIG. 2, Larsen et al. 2017). In expanded T cells, the majority of cells (if not all) are effector memory cells.

The short duration of such effects in the prior art makes it difficult to exploit beneficial reversible effects on immune cells in adoptive cell therapy, where typically the immune cells are treated outside the body before being infused into the patient. By the time the cells are treated and introduced into the patient, the effect has diminished and/or will not be present for a sufficient time to enhance the cell therapy.

However, the inventors have demonstrated that the effect of the Nrf2 activator lasts for a sufficiently long time (i.e. at 48 hours, 72 hours or longer) to enable effective cell therapy. The skilled person will understand that this duration will provide adequate time for administration of the NK cells and/or T cells to a patient and anti-cancer activity. The present inventors have observed that killing of tumour cells by cytotoxic NK cells and T cells can typically be detected within a couple of hours, as measured by ex vivo cytotoxicity assays. Furthermore, in vivo mouse experiments have shown that NK cells can kill rapidly. Hayakawa et al. showed that dendritic cells (target cells) can be killed within 8 h after intravenous injections of NK cells (FIG. 1 of Hayakawa et al., 2004). VahIne et al. 2010 indicates that tumour cells could be fully eliminated within 40 hours after intravenous injection of NK cells. Hence, the duration for which the inventors have identified the beneficial effects on NK cell and/or T cell activity and survival are within a clinically relevant range that can lead to optimal tumour elimination.

Importantly, the beneficial effects on NK cell and/or T cell activity and/or survival observed by the inventors are not permanent and reverse after an appropriate duration and do not permanently affect the NK cell and/or T cell. This provides major benefits over other alternative methods of manipulating NK cells and/or T cells, such as gene editing techniques (e.g. CRISPR-based approaches) which permanently modify the NK cells and/or T cells. Permanent changes to NK cells and/or T cells can lead to the NK cells and/or T cells being overactive in the body and/or causing undesirable side-effects. These drawbacks are avoided by the present invention.

Overall, the duration of the effects demonstrated by the present inventors fall within a clinically useful timeframe for adoptive cell therapy and cancer therapy, but without the drawbacks of a permanent modification and are therefore highly desirable.

In some embodiments, the one or more NK cell and/or T cell may have a longer-term advantage (e.g. improved survival) relative to one or more NK cell and/or T cell that has not been treated with an activator of Nrf2. “Longer-term” may include a duration of months or years.

Furthermore, contacting NK cells and/or T cells with an activator of Nrf2 offers other benefits. For example, it is a simple and easy to use the clinic. Furthermore, the activators of Nrf2 auranofin and DMF are FDA and/or EMA-approved. CRISPR/gene-editing approaches are more complex and may not always work.

In an embodiment of the invention, the cancer to be treated is a bladder cancer, bone cancer, breast cancer, colon cancer, cervical cancer, rectal cancer, endometrial cancer, oesophageal cancer, ovarian cancer, gastric cancer, kidney cancer, leukaemia, liver cancer, lung cancer, skin cancer (including basal cell carcinoma, squamous cell carcinoma, melanoma, non-skin located uveal melanoma and/or mucosal melanoma), lymphoma, pancreatic cancer, prostate cancer, testicular cancer and/or thyroid cancer.

In an embodiment of the invention, the cancer is a solid tumour, optionally wherein there is a high level of one or more stress, such as oxidative stress, present in the solid tumour microenvironment.

The skilled person will appreciate that what a “high level” of stress, such as oxidative stress, for the one or more NK cell and/or T cell will be dependent on the patient and/or donor.

In an embodiment of the invention, the patient in need thereof has a viral infection.

In an embodiment of the invention, the NK cell and/or T cell is contacted with an activator of Nrf2 at a concentration between 0.1 μM and 500 M, for example wherein the concentration of the activator of Nrf2 is 0.1 μM, 0.25 μM, 0.5 μM, 0.75 μM, 1 μM, 1.5 μM, 2 M, 2.5 μM, 3 μM, 4 M, 5 μM, 7.5 μM, 10 μM, 15 μM, 20 μM, 25 μM, 30 μM, 40 μM, 45 μM, 50 μM, 60 μM, 70 μM, 80 M, 90 μM, 100 M, 150 μM, 200 M, 250 μM, 300 μM, 350 μM, 400 μM, 450 μM or 500 μM.

In an embodiment of the invention, the NK cell and/or T cell is contacted with an activator of Nrf2 at a concentration between 0.1 μg/ml and 500 μg/ml, for example wherein the concentration of the activator of Nrf2 is 0.1 μg/ml, 0.25 μg/ml, 0.5 μg/ml, 0.75 μg/ml, 1 μg/ml, 1.5 μg/ml, 2 μg/ml, 2.5 μg/ml, 3 μg/ml, 4 μg/ml, 5 μg/ml, 7.5 μg/ml, 10 μg/ml, 15 μg/ml, 20 μg/ml, 25 μg/ml, 30 μg/ml, 40 μg/ml, 45 μg/ml, 50 μg/ml, 60 μg/ml, 70 μg/ml, 80 μg/ml, 90 μg/ml, 100 μg/ml, 150 μg/ml, 200 μg/ml, 250 μg/ml, 300 μg/ml, 350 μg/ml, 400 μg/ml, 450 μg/ml or 500 μg/ml.

In a preferred embodiment of the invention, the activator of Nrf2 is auranofin and wherein the NK cell and/or T cell is contacted with auranofin at a concentration of 1 μM, 5 μM, 10 μM or 25 μM, 0.25 μg/ml, 0.5 μg/ml or 1 μg/ml.

In a preferred embodiment of the invention, the activator of Nrf2 is sulforaphane and wherein the NK cell and/or T cell is contacted with sulforaphane at a concentration of 2.5 μM, 5 μM, 10 μM or 25 μM.

In a preferred embodiment of the invention, the activator of Nrf2 is dimethyl fumarate (DMF) and wherein the NK cell and/or T cell is contacted with dimethyl fumarate (DMF) at a concentration of 5 μM, 10 μM or 25 μM.

In an embodiment of the invention, the amount of one or more NK cell and/or T cell present is between 1×10⁴ and 1×10¹⁵ cells, for example 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵ cells, preferably 1×10⁶ cells. The number of one or more T cell and/or NK cells present is between 1×10⁴ and 1×10⁸ cells, for example 1×10⁴, 1×10⁵, 1×10⁶, 1×10⁷, 1×10⁸ cells, preferably 1×10⁶ cells or 1×10⁷ to 1×10¹² cells, such as 1×10⁸ to 5×10⁹ cells, such as 1×10⁹ to 5×10⁹ cells, such as 1×10⁸ to 5×10¹⁰ cells, such as 1×10⁹ to 5×10¹¹ cells.

In an embodiment according to any aspect of the invention, the one or more NK cell and/or T cell is a tumour infiltrating lymphocyte (TIL) and/or a chimeric antigen receptor T cell (CAR-T cell).

In an embodiment of the invention, the one or more NK cell and/or T cell is administered in combination with one or more further therapeutic agent, particularly wherein the one or more further therapeutic agent is an anti-cancer therapeutic agent, such as an anti-cancer antibody, an anti-CD20 agent (e.g. Ofatumumab or Rituximab), a chimeric antigen receptor T cell (e.g. a CD19+ chimeric antigen receptor T cell) and/or non-activated monocytes, oncolytic viruses, or bi-specific antibodies which have the property of “linking” T cells or NK cells to molecules on the tumour cells thus enhancing the tumour elimination.

By “administered in combination” we include that the one or more NK cell and/or T cell and the further therapeutic agent are present within the body of the patient simultaneously. The one or more NK cell and/or T cell and the further therapeutic agent may be present in the same composition or may present in separate compositions which are administered simultaneously or sequentially.

By “further therapeutic agent” we include all known therapeutic agents and in particular all anti-cancer agents or anti-angiogenesis agents (as defined earlier in the application). It will be appreciated that the further therapeutic agent may be one that is known to be suitable for combating the disease towards which the treatments of the invention are directed. The further therapeutic agent may be a checkpoint inhibitor, such as PD1/PD-L1 checkpoint inhibitor and/or a CTLA-4 checkpoint inhibitor.

In particular, it is preferable if the further therapeutic agent and or anti-cancer agent is an anti-cancer antibody, an anti-CD20 agent (e.g. Ofatumumab or Rituximab), a chimeric antigen receptor T cell/NK cell (e.g. a CD19+ chimeric antigen receptor T cell) and/or non-activated monocytes.

In a further aspect, the invention provides a method for inducing and/or increasing NK cell and/or T cell activity wherein the method comprises a step of contacting one or more NK cell and/or T cell with an activator of Nrf2 ex vivo, wherein the method further comprises a step of administering the one or more NK cell and/or T cell to a patient in need thereof.

In an embodiment, the patient in need thereof has cancer, optionally wherein the cancer is characterised by the presence of one or more stress, such as oxidative stress, hypoxia, reoxygenation, starvation.

In an embodiment, the NK cell and/or T cell activity is selected from one or more of:
i) anti-cancer or anti-tumour activity;
ii) production and/or release of cytokines;
iii) production and/or release of IFN-γ;
iv) effector function in tumour and/or spheroid tumour structures;
v) specific lysis of a target cell, for example a tumour and/or a cancer cell; and/or
vi) degranulation and/or capacity to degranulate;
vii) ability to regulate and/or influence other immune cell types, such as Dendritic cells, macrophages or other monocyte/myeloid cell types, or other lymphocyte cell (e.g. NK mediated regulation of T cell activity and vice versa).

In an embodiment, the one or more NK cell and/or T cell is contacted with an activator of Nrf2 for up to 48 hours, optionally between 0.5 and 24 hours, and preferably for 0.1 hours, 0.2 hours, 0.3 hours, 0.4 hours, 0.5 hours, 0.75 hours, 1 hour, 2 hours, 3 hours, 4 hours, 5 hours, 6 hours, 7 hours, 8 hours, 9 hours, 10 hours, 11 hours, 12 hours, 14 hours, 16 hours, 18 hours or 24 hours.

In a preferred embodiment, the effect on NK cell and/or T cell activity is present for at least 24 hours, at least 30 hours, at least 36 hours, at least 42 hours, at least 48 hours, at least 54 hours, at least 60 hours, at least 66 hours, and/or at least 72 hours after the contacting step and/or after the activator of Nrf2 is removed and/or washed away from the one or more NK cell and/or T cell.

The cells, stresses, activators of Nrf2, patients, cell activities, steps and durations of the methods may be as defined in any of the earlier aspects.

In a further aspect, the invention provides a population of NK cells and/or T cells that has been contacted with an activator of Nrf2, characterised in that the population comprises a higher frequency of CD3ζ+ cells.

In a further aspect, the invention provides a population of NK cells and/or T cells that has been contacted with an activator of Nrf2, characterised in that the population comprises a higher frequency of CD3ζ+/CD16+ cells.

In a further aspect, the invention provides a population of NK cells and/or T cells that has been contacted with an activator of Nrf2, characterised in that the population comprises a higher frequency of CD16+ cells.

The higher frequency of CD16+ and/or CD3ζ+/CD16+ and/or of CD37+ cells may be relative to a population of NK cells and/or T cells that have not been contacted with an activator of Nrf2. The population of NK cells and/or T cells may be produced by the methods defined in any of the earlier aspects.

A further aspect relates to the population of NK cells and/or T cells for use in therapy.

A further aspect relates to the population of NK cells and/or T cells for use in the treatment and/or prevention of cancer. The cancer may be as defined for any of the previous aspects.

A further aspect relates to a method of treating and/or preventing cancer comprising a step of administering the population of NK cells and/or T cells to a patient in need thereof. The method may include features of the methods of any of the previous aspects.

A further aspect relates to the use of the population of NK cells and/or T cells in the manufacture of a medicament for use in therapy and/or medicine, including for use in the treatment and/or prevention of cancer. The cancer may be as defined for any of the previous aspects.

The invention provides a use, a method or an activator of Nrf2 substantially as described herein with reference to the accompanying claims, description and/or figures.

The invention will now be described by reference to the following Figures and Examples.

### LIST OF FIGURES

FIG. 1: AUF, SUL and DMF pre-treatment counteracts the suppressive effect of H₂O₂ on lymphocyte function. Assessment of Expanded NK cells and patient derived TIL for their effector function, with and without pre-treatment with a Nrf2 activating compound, upon exposure to oxidative stress was measured by killing/specific lysis, degranulation (CD107a expression) or cytokine release (IFNγ production). A-D, G-I Auranofin pre-treated NK cells were analyzed for their ability to perform effector functions upon oxidative stress. A Lysis of K562 target cells upon increasing H₂O₂ concentrations (one representative experiment). B Lysis of K562 cells (E:T 9:1) by NK cells with and without AUF pre-treatment after exposure to H₂O₂, n=3, paired t-test. C Effect shown by linear regression. D Lysis of K562 target cells. When indicated, catalase were added to control NK cells before incubation with H₂O₂. E IFNγ release by NK cells with and without AUF pre-treatment after exposure to H₂O₂, n=4. F Frequency of alive NK cells (% of single cells) 4 h after H₂O₂ treatment, n=3. G Degranulation by NK cells stimulated with K562 target cells. NK cell mediated lysis of KASUMI-1 (H) or THP-1 (I). J-K Lysis of K562 cells by NK cells pre-treated with either SUL (J) or DMF (K). L-N Assessment of autologous TIL-tumour recognition by IFNγ release upon H₂O₂ treatment. TIL were pre-treated with either AUF (L), SUL (M) or DMF (N). Statistic analysis: D, G-K paired t-test. L unpaired t-test. ***p<0.001, **p<0.01, *p<0.05. Each data point represents one NK cell donor or one TIL experiment. Effector: target ratio 9:1 (A, C-D, G-K) or 4:1 (L-N). O ANRU TIL were pre-treated with AUF, exposed to indicated concentration of H₂O₂, followed by 24 h co-culture with ANRU tumour cells and thereafter stained for live cells and analyzed by flow cytometry. Plot show frequency of alive CD3+ T cells (% of single cells). ***p<0.001, **p<0.01, *p<0.05. P Lysis of KADA tumour cells by autologous TIL, with and without AUF pre-treatment, after H₂O₂ treatment, n=3, unpaired t-test.

FIG. 2: The effect of AUF, SUL and DMF is dependent on Nrf2 activation. Kinetic quantification of transcription of classical Nrf2 target genes in NK cells and TIL after pre-treatment with indicated Nrf2 activating compounds using qPCR or assessing effector function by specific lysis of K562 target cells with and without a Nrf2 inhibitor. A-E For NK cells, kinetic quantification of indicated genes after pre-treatment with either AUF (A), SUL (B), DMF (C) or DMSO (D). E Kinetic study of the effect of AUF pre-treatment on K562 target cell lysis upon exposure to H₂O₂, E:T ratio 9:1. F Kinetic quantification of indicated genes in TIL after AUF pre-treatment. G-I Lysis of K562 cells by NK cells pre-treated with AUF (G), SUL (H), DMF (I) with or without the Nrf2 inhibitor ML385. Quantification of gene expression; values represent the mean fold change gene expression compared to untreated cells. For NK cell donors; 4 (AUF) or 3 (DMF, SUL, DMSO), three TIL donors (KADA, BEHA, ANRU). Statistic; E, G-H Paired t-test, ***p<0.001, **p<0.01, *p<0.05. Each data point represents one NK cell donor.

FIG. 3: A IFNγ release by NK cells with and without AUF pre-treatment for 18 h or 24 h, n=1. B IFNγ release by NK cells pre-treated with AUF either for 18 h continuously or pulse treated for 30 min followed by 17.5 h incubation without AUF, n=4. Overlapping datapoints with FIG. 1E. C Fold change gene expression of Nrf2 target genes in KADA, ANRU and BEHA TIL (compared to untreated). D IFNγ release by expanded healthy donor CD8+ T cells with and without AUF pre-treatment for indicated time. E Lysis of K562 by NK cells pre-treated with either DMSO, AUF or a combination of AUF and ML385. F Lysis of K562 cells by NK cells pre-treated with DMSO+/−the Nrf2 inhibitor ML385.

FIG. 4: Auranofin pre-treated NK cells and TIL show decreased intracellular ROS levels and increased effector function after H₂O₂ treatment up to 72 h after compound removal. NK cells or TIL were pre-treated with Auranofin for 18 h, cultured without AUF for the indicated timepoints and thereafter challenged with H₂O₂ at indicated concentrations. For NK cells, A-D intracellular ROS levels and E-H lysis of K562 target cells (E:T 3:1) were measured in parallel at 0 h (A, E), 24 h (B, F), 48 h (C, G) or 72 h (D, H) after AUF removal. I Representative histograms showing intracellular ROS (CellROX) in NK cells after H₂O₂ treatment. J Plot showing Spearman correlation between intracellular ROS levels and NK cell function with paired data points from A-D, E-H. K Intracellular ROS levels in TIL 0-72 h after AUF treatment relative to control cells. L-O For TIL, lysis of autologous tumour cells were measured at 0 h (L), 24 h (M), 48 h (N) or 72 h (O) after AUF removal. Statistic analysis: A-D, H-K 2-way ANOVA, ***p<0.001, **p<0.01, *p<0.05. Each data point represents one NK cell/TIL donor.

FIG. 5: Auranofin protects NK cells from monocyte-derived ROS. A-B Luminol-based detection of monocyte-derived ROS. A Representative plot showing cell-number dependent ROS production by monocytes. B ROS production measured by Luminescence, indicated H₂O₂ concentrations was analyzed in parallel as comparison. C Representative histograms showing intracellular ROS levels (CellROX) in control NK cells after co-culture with monocytes. D Intracellular ROS levels and E lysis of K562 cells (E:T 9:1) by AUF pre-treated NK cells upon co-culture with autologous monocytes. Statistic analysis: D-E paired t-test, ***p<0.001, **p<0.01, *p<0.05. Each data point represents one NK cell donor.

FIG. 6: A Intracellular ROS levels in NK cells after co-culture with autologous monocytes in the presence of 100 U/mL catalase. B Lysis of K562 cells by NK cells after co-culture with autologous monocytes in presence of catalase. One data point represents one NK cell donor.

FIG. 7: A Characterization of CD19 and CD20 expression in RAJI and N6/ADR cell lines using flow cytometry. B Lysis of RAJI cells (E:T 9:1) by NK cells in the presence (+) or absence (−) of Ofatumumab (n=5) or Rituximab (n=6). C-F Phenotype analysis of NK cell surface markers with and without AUF pre-treatment and with and without H₂O₂ exposure at indicated concentration. C+D show frequency of positive NK cells (%) while E+F show geometric mean fluorescent intensity (gMFI). One data point represents one NK cell donor. Statistic, paired T-test. G Intracellular ROS (CM-H₂DCFDA) staining in ANRU tumour cells grown in 2D or as spheroids for three days. H Quantification of ROS in ANRU tumour cells shown in G. I Luminol-based detection of ROS produced by PMA stimulated ANRU monocytes. J Representative live cell images showing Caspase 3/7 activation in ANRU spheroids after 48 h co-culture with autologous TIL. TIL were cultured with monocytes prior to being added to the spheroid. K Lysis of ANRU tumour cells by autologous TIL pre-exposed to activated autologous monocytes.

FIG. 8: Auranofin pre-treatment can be used in combination with existing treatment options for hematological and solid tumours. A-C NK cells were pre-treated with and without AUF and then challenged with H₂O₂. A Lysis of RAJI cells in the presence of Ofatumumab or Rituximab (E:T 9:1). B NK cell recognition of Rituximab coated RAJI cells measured by degranulation (CD107a) C Frequency of CD16+D-E CD19-directed CAR T-cell lysis of RAJI (D) or N6/ADR (E) tumour cells (E:T 15:1). F-G ANRU TIL killing of autologous tumour cell spheroids measured by caspase 3/7 activation during live cell imaging. F Representative images after 48 h co-culture. G Quantification of caspase activation in spheroids at indicated time. H Confocal images of ANRU spheroids showing CD8⁺ TIL infiltration after 48 h co-culture. I Quantification of T cell infiltration into ANRU spheroids using flow cytometry, n=6. J Experimental design for I-K. ANRU TIL, with or without AUF pre-treatment, were co-cultured with autologous monocytes, unstimulated or stimulated, before assessment of their functional capacity by IFNγ release and killing of autologous tumour cells grown in 2D and 3D. K ANRU TIL were co-cultured with unstimulated or activated autologous monocytes and then assessed for their capability to kill ANRU tumour spheroids, measured by caspase activation. L-M ANRU TIL responses against autologous ANRU tumour spheroids or 2D grown tumour cells measured by caspase activation (L) or IFNγ release (M), respectively. N Potential applications for AUF or similar compounds to improve adoptive cell therapy. A-E Each data point represents one NK cell or CAR T cell donor. G Represents mean of four independent experiments. Statistic analysis: A-E Paired t-test. I, K, L One-way Anova with Turkey's multiple comparisons test. ***p<0.001, **p<0.01, *p<0.05.

FIG. 9: Targeting intrinsic antioxidant pathways in NK cells and TIL preserves their efficient anti-tumor responses after H₂O₂ exposure. NK cells or TIL were pre-treated with indicated Nrf2 activating compound, exposed to indicated concentration of H₂O₂ and co-cultured with indicated tumor target. Tumor target recognition was measured by target lysis via 51Cr assay or cytokine production using flow cytometry. A: Lysis of K562 cells by NK cells pre-treated with AUF or DMSO and the control cells were analyzed with and without pre-treated with NAC for 1 h prior to the H₂O₂ treatment, n=5. B-C: AUF pre-treated and control NK cells were exposed to H₂O₂ at indicated concentration, co-cultured with K562 tumor cells for 6 h, and tumor recognition was measured by degranulation and cytokine production (CD107a and IFNγ expression, respectively) by flow cytometry, n=4. Statistical analysis: paired t-test. ***p<0.001, **p<0.01, *p<0.05. Each data point represents one NK cell donor. Error bars in bar plots show mean with SD. Box plots show the median with error bars from minimum and maximum point. (Note that FIG. 9B corresponds to FIG. 1G and is presented again here as a reference).

FIG. 10: AUF pre-treatment of TIL do not induce regulatory T cells: KADA TIL were pre-treated with AUF (0.5 μg/mL), washed and cultured for indicated durations before quantification of CD4+ T cells (A) and Tregs (B) using flow cytometry (t; hours). Tregs were defined as CD3+CD4+ FoxP3+. Directly after the treatment, TIL were stimulated for six hours with PMA/Ionomycin or autologous tumor cells and then stained for intracellular cytokines IL-10 (C) and TGFβ (D).

### EXAMPLES

### Example 1

### Summary

Adoptive cell therapy using cytotoxic lymphocytes has proven to be efficient as immunotherapy against solid and hematological cancers. However, the tumour microenvironment has been shown to potentially be very hostile, including production of elevated levels of reactive oxygen species (ROS), which can impair NK and/or T cell function. The inventors have surprisingly found that human cytotoxic lymphocytes can be made more resistant towards oxidative stress via compound induced Nrf2 activation. Pre-treatment of NK cells and tumour infiltrating lymphocytes (TIL) with a low dose of the FDA-approved compound auranofin reduced accumulation of intracellular ROS and thereby preserved their antitumoural activity despite high H₂O₂ levels. Furthermore, comparable results were obtained for auranofin pre-treated NK cells and TIL upon co-culture with autologous activated monocytes. Analysis of transcription of classical Nrf2 target genes and the usage of a Nrf2 inhibitor showed that the increased resistance towards oxidative stress was Nrf2 dependent. In addition, auranofin pre-treatment improved tumour killing by CD19 directed CAR T cells and increased elimination of spheroid grown tumour cells by autologous TIL after exposure to H₂O₂ or autologous monocyte derived ROS, respectively. These findings indicate that Nrf2 activation in human cytotoxic lymphocytes may be used to improve the cell product used for adoptive cell therapy to enhance the efficacy of existing immunotherapies.

### Introduction

Adoptive cell therapy (ACT) using tumour infiltrating lymphocytes (TIL), natural killer (NK) cells or genetically modified NK- or T cells has proven very effective as immunotherapies to treat patients with various advanced malignancies. Regardless of the promising results, there are still hurdles to overcome and room for improvement. For example, the infused cells need to persist in the patient long enough to eliminate the cancer cells meanwhile surviving the hostile tumour microenvironment (TME).

Cancer cells and tumour-infiltrating immune cells, such as myeloid-derived suppressor cells (MDSC), contribute in creating a hostile and immune suppressive TME, especially by the production of reactive oxygen species (ROS) (Schmielau and Finn, 2001).

Reactive Oxygen Species (ROS) are chemically reactive oxygen-containing molecules, such as superoxide radicals (O₂), hydroxyl radicals (OH) or hydrogen peroxide (H₂O₂). Intracellularly produced ROS play an important role as secondary messengers in cellular signalling cascades (Schieber and Chandel, 2014; Sies and Jones, 2020). The reduction-oxidation (redox) balance regulates many biological processes, including immune responses (Mehrotra et al. 2009). There are two major reductive enzyme systems maintaining redox homeostasis, namely the glutathione (GSH) and Thioredoxin (Trx) systems, utilizing NADPH as reducing equivalent (Miller et al. 2018).

Bursts of ROS produced by NADPH oxidases are essential to mediate innate immune cell functions against invading microbes and as anti-tumoural response (Sies and Jones, 2020). However, sustained elevated ROS levels have been shown to diminish the immune response by inducing poor effector functions or cell death in T and NK cells (Norell et al., 2009; Harlin et al., 2007). Early studies showed that ROS produced by autologous monocyte in the TME suppressed NK- and T cell function and their capability to response to cytokine activation (Hellstrand, 2002). Various studies have shown that increased ROS levels leads to downregulation of the TCR/CD3 complex in T cells and the low-affinity Fc receptor FcγRIII (also known as CD16) on NK cells, resulting in reduced cytotoxic capacity (Otsuji et al. 1996; Kono et al. 1996). One way to combat this deleterious effect on the immune system is to counteract and/or reduce the accumulation of ROS in the TME. It has been shown that addition of histamine (Ceplene®) can inhibit ROS production by the monocytes and thereby protect NK cells and T cells from ROS mediated suppression in patients with acute myeloid leukemia.

The inventors have surprisingly identified another mechanism by which the harmful effects of ROS on the immune system can be counteracted. This mechanism targets the antioxidant system within the cytotoxic lymphocytes to increase their inherent resistant to oxidative stress in the TME. In this context Nrf2 (nuclear factor E2-related factor 2) is of interest as a transcription factor controlling a wide range of downstream targets that can help the cells obtain increased resistance to ROS accumulation. Nrf2 is a transcription factor that is regulated by the constitutively expressed Keap1 (Kelch-like ECH-associated protein1). In non-stressed cells, the Kelch domains of Keap1 bind to Nrf2 and promotes its degradation by the 26S proteasome. During targeting of Keap1 with electrophiles, upon oxidative stress, or upon inhibition of thioredoxin reductase 1 (TrxR1), Keap1 loses the capacity to tightly bind Nrf2, thereby allowing Nrf2 to enter the nucleus (Cebula et al., 2015; Lei et al., 2016). Activated Nrf2 binds to antioxidant response elements (ARE) in target gene promoters thereby initiating transcription of genes encoding detoxifying enzymes, cytoprotective proteins and/or antioxidant proteins such as NAD(P)H quinione oxidoreductase 1 (NQO1), glutathione S transferases (GST) or heme oxygenase 1 (HO-1).

Auranofin (AUF) is a gold (I)-containing phosphine compound that was approved in 1985 to treat patients with Rheumatoid arthritis and is a strong activator of Nfr2 as well as inhibitor of TrxR1. High doses of AUF easily become toxic and induces cell death in cancer cells, with AUF currently also being evaluated for anti-cancer therapy in a number of clinical trials.

The inventors demonstrated that human cytotoxic lymphocytes gain increased resistance towards oxidative stress and improved antitumoural efficacy after contact with activators of Nrf2, such as AUF. Pre-treatment of NK cells, TIL and CAR T cells with AUF rendered these cells more resistant to H₂O₂ as well as monocyte-derived ROS, resulting in increased tumour elimination and cytokine release. The pharmacological activation of intrinsic antioxidant pathways could be a promising strategy to protect the effector functions of cytotoxic lymphocytes with a strong anti-tumour capacity, which can thereby potentially be used to improve the results of adoptive cell therapy.

Targeting Intrinsic Antioxidant Pathways in NK Cells and TIL Improves Anti-Tumour Responses after H₂O₂ Exposure

Auranofin (AUF, Ridaura®), an inhibitor of the selenoprotein thioredoxin reductase (TrxR) and is a strong activator of Nrf2. The inventors investigated if pre-treating NK- and T cell with AUF could increase their antitumoural efficacy in stress conditions, such as conditions of oxidative stress.

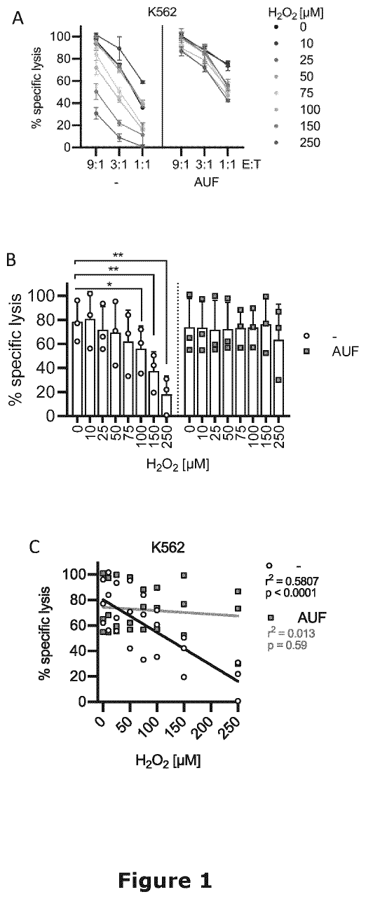

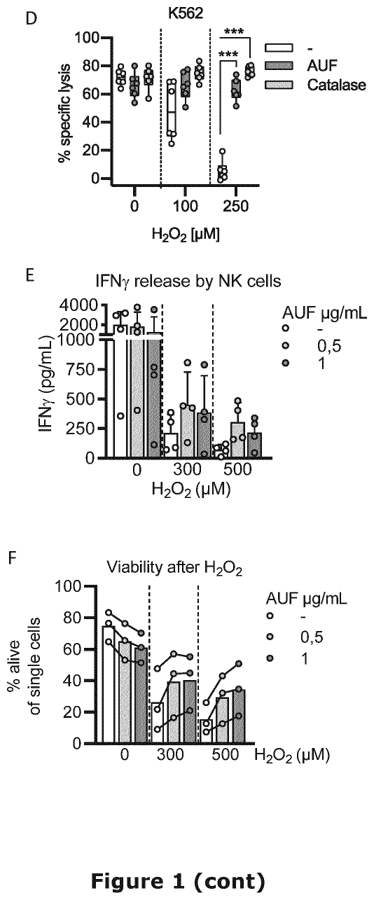

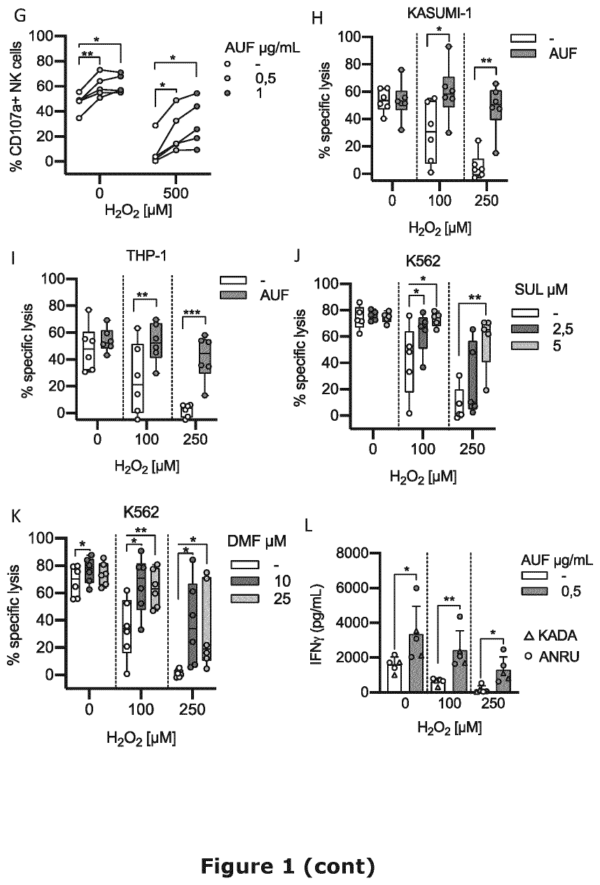

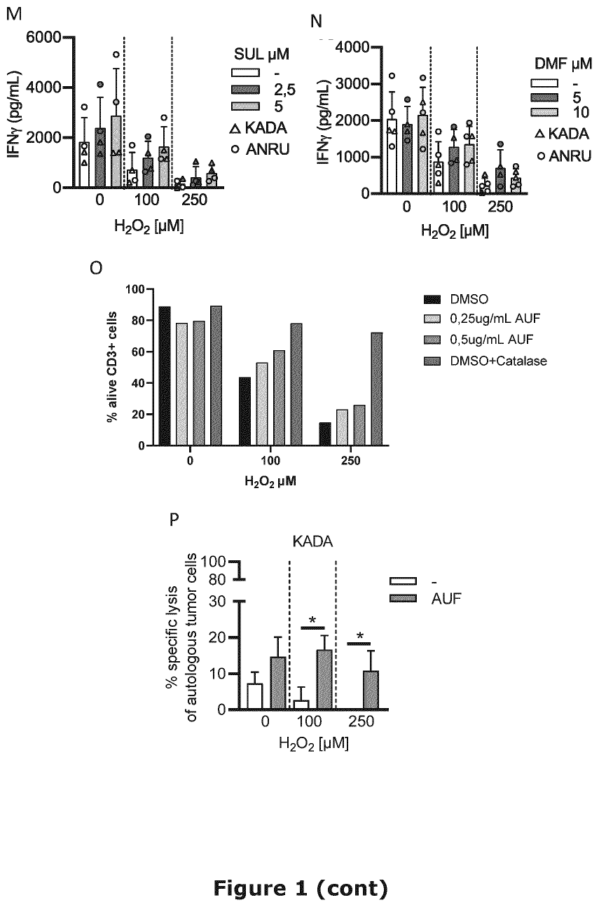

Healthy donor NK cells or patient derived tumour-infiltrating lymphocytes (TIL) were expanded with protocols compatible with those utilised for ATC, and the cytotoxicity of the expanded cells was analysed. The NK cells were exposed to increasing levels of hydrogen peroxide (H₂O₂) and subsequently co-cultured with the NK cell sensitive human immortalised K562 myelogenous leukemia cells. Exposing expanded NK cells to H₂O₂ mediated oxidative stress significantly decreased their ability to kill K562 target cells in a dose-dependent manner (FIG. 1A and FIG. 1B). However, this effect was counteracted by AUF pre-treatment of NK cells even after exposure to high (250 μM) concentrations of H₂O₂ (FIG. 1C).

In addition, AUF pre-treated NK cells also had an improved viability, cytokine release (IFNγ) capacity following exposure to H₂O₂ compared to untreated NK cells (FIGS. 1A and 1C-F). Furthermore, the addition of catalase to the control group, an enzyme efficiently converting H₂O₂ to H₂O and O₂, improve the function of the control NK cells in a comparable way to AUF pre-treatment (FIG. 1D). Thus, we conclude that AUF pre-treatment of NK cells provides a strong protective effect against ROS, such as H₂O₂.

Auranofin pre-treated NK cells also displayed an increased degranulation capacity after co-culture with K562 target cells, as measured by CD107a expression by flow cytometry. This occurred both with and without induction of oxidative stress (FIG. 1G). Concordant with the killing of K562 target cells, AUF pre-treated NK cells also displayed a significantly increased killing of two different acute myelogenous leukemia (AML) cell lines, KASUMI-1 and THP-1. AUF pre-treatment of NK cells intrinsically enhances NK cell degranulation and results in a robust and efficient protection of the antitumoural capacity against cellular stresses, such as ROS, independently of the tumour target (FIG. 1H-I).

The inventors have demonstrated that treatment with other activators of Nrf2, such as Dimethyl Fumarate (DMF) or Sulforaphane (SUL), results in similar protective effects on NK cells and T cells. NK cells pre-treated with SUL and DMF indeed resulted in comparable protective effects as AUF (FIG. 1J-K).

To further evaluate the effect of targeting Nrf2 in cell products used for ACT, the effect of pre-treating TIL was explored. TIL from two melanoma patients, KADA and ANRU, were pre-treated with either AUF, SUL or DMF. Their resistance to H₂O₂ was then investigated by measuring cytokine (IFNγ) production. TIL pre-treated with any of the compounds yielded increased recognition of their autologous tumour cells upon exposure to H₂O₂, as compared to the control group (FIG. 1L-O). Comparably, specific lysis of the autologous tumour cells by TIL was clearly higher upon AUF pre-treatment, also in the absence of ROS (FIG. 1P). In concordance to NK cells, AUF pre-treated ANRU TIL had improved viability after exposure to H₂O₂. Thus, both expanded NK cells and TIL cell products display improved fitness, survival and activity in H₂O₂ exposed conditions after being contacted with an activator of Nrf2.

Activation of typical Nrf2-dependent transcription patterns in human lymphocytes

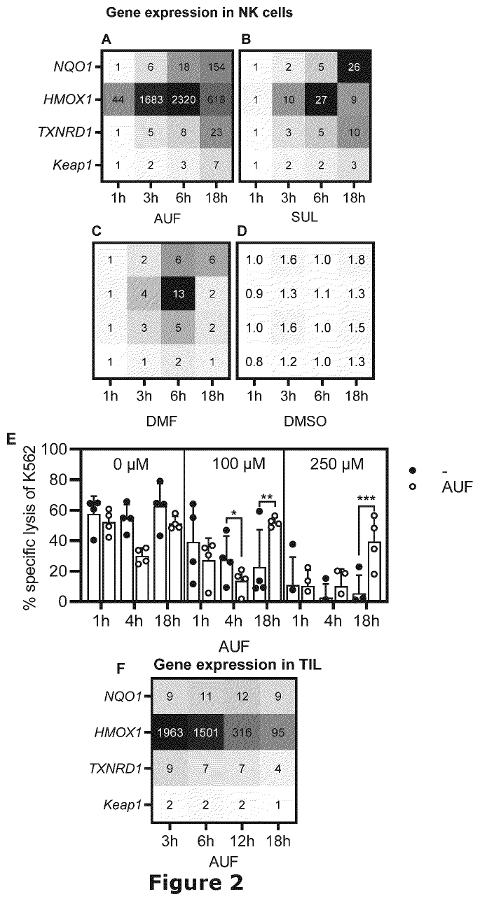

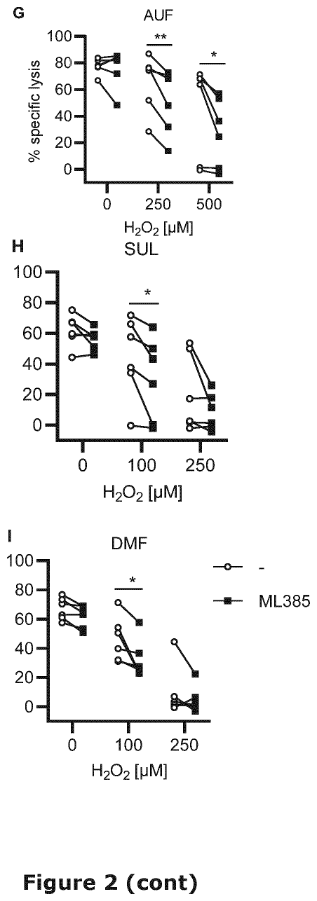

In order to validate that contacting the NK cells and/or T cells with AUF, DMF and SUL does indeed activate the typical Nrf2-dependent antioxidant pathways in lymphocytes typically described for cancer cells, transcription of the classical Nrf2-targets NQO1, HMOX1, TXNRD1, as well as Keap1, by qPCR was quantified (FIG. 2). Without being bound by a theory, the increased transcription of a number of protective enzymes could explain the observed increased resistance against H₂O₂.

Displaying the expression levels as fold changes compared to untreated NK cells or TIL, it was clear that for NK cells AUF pre-treatment triggered a pronounced upregulation of HMOX1 already at 1 h while the expression of the other target genes more gradually increased over time (FIG. 2A). Sulforaphane and DMF had a comparable influence on these Nrf2 target genes (FIG. 2B-C). No upregulation was seen in DMSO treatment controls, showing that the observed effect was compound specific (FIG. 2D).

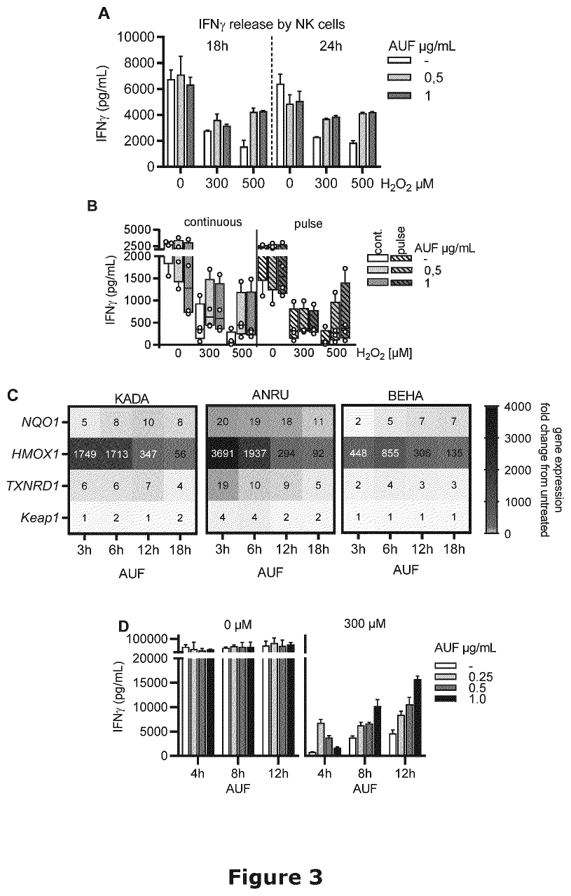

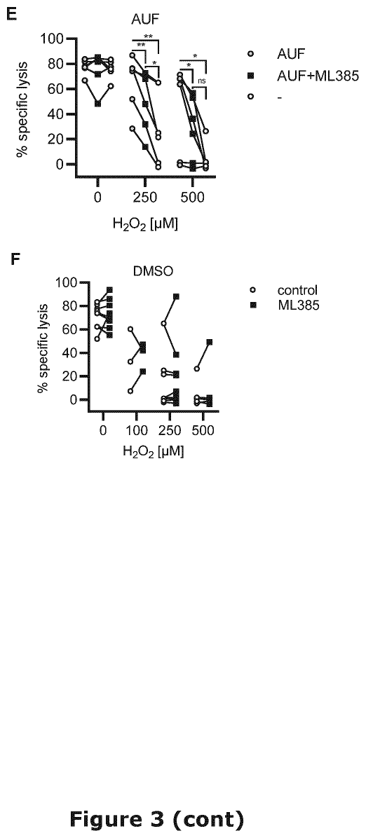

Based on the gene expression data, the effect of treatment-time was investigated. The most pronounced protection of the functional cytotoxicity capacity occurred around 18 h after the start of AUF pre-treatment for the NK cells (FIG. 2E). There was no difference comparing 18 h and 24 h pre-treatment or a 30 min AUF pulse followed by 17.5 h incubation (FIG. 3A-B). Auranofin pre-treatment also induced a robust activation of the Nrf2 target genes in TIL from three melanoma patients, KADA, ANRU and BEHA (FIG. 2F and FIG. 3C). The responses in TIL were faster than in NK cells, with the HMOX1 expression peaking at 3 h compared to the peak observed at 6 h in NK cells. In agreement with this finding, a protective effect was observed in T cells after 8-12 h of AUF pre-treatment (FIG. 3D).

To confirm that activation of Nrf2 was responsible for the increased resistance towards H₂O₂ in cytotoxic lymphocytes, a Nrf2 inhibitor was used. For this, NK cells were pre-treated with either AUF, SUL or DMF in combination with the Nrf2 inhibitor ML385. Lysis of K562 was significantly reduced by ML385 when cells were exposed to H₂O₂ (FIG. 2G-I). This suggests that inhibition of Nrf2 counteracts the protective effects. However, despite addition of ML385, lysis was increased by AUF pre-treated NK cells compared to the untreated controls (FIG. 3E). This could either indicate additional mechanisms of action or an incomplete inhibition of Nrf2 activity. Importantly, no effects of ML385 were observed in DMSO pre-treated control NK cells (FIG. 3F).

Together, these findings demonstrate that activating Nrf2 pathway can be used improve lymphocyte resistance against oxidative stress and thus their ability to exert cytotoxic functions.

Increased Anti-Tumour Activities Remain Up to 72 h after AUF Pre-Treatment.

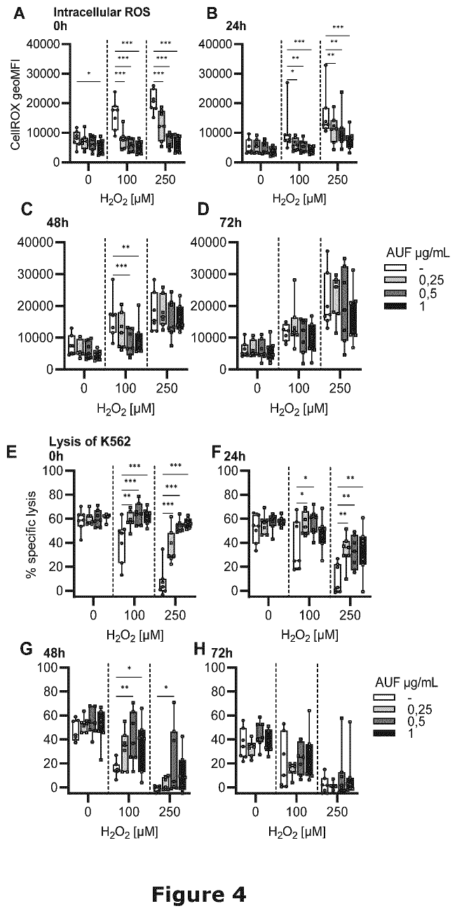

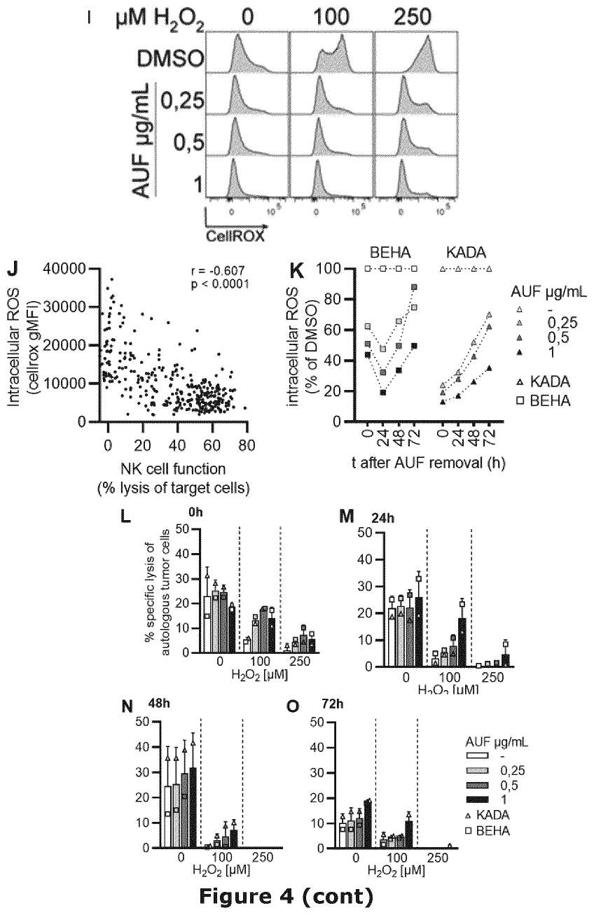

It is important for the cells used for ATC to persist a sufficient time following infusion to the patient to be able to encounter and eliminate the tumour. Experiments were carried out to assess the duration of the protective effects of activators of Nrf2 (such as AUF) on NK cells and/or T cells. NK cells and TIL were contacted with AUF and intracellular ROS levels and target cell lysis, with or without addition of H₂O₂, were investigated at 0 h, 24 h, 48 h and 72 h after AUF removal (FIG. 4). Intracellular ROS levels were assessed using flow cytometry and a cell-permeant dye that increases in fluorescence intensity upon oxidation. In untreated NK cells, fluorescence was gradually increasing with H₂O₂ concentrations (FIG. 4A-D, I), which also correlated with decreased function as seen by impaired target cell lysis (FIG. 4E-H). Of note, AUF pre-treated NK cells displayed significantly lower intracellular fluorescence of the ROS indicator. This correlated with significantly improved effector functions up to 48 h after AUF removal (FIGS. 4A-C, I and E-G). Conversely, 72 h after AUF removal the protective effect had diminished as no differences in fluorescence or effector functions could then be detected between AUF pre-treated NK cells and controls (FIGS. 4D and H). Even without addition of H₂O₂, the AUF pre-treatment resulted in significantly reduced intracellular ROS levels. This indicates that AUF treatment also can decrease the endogenous production of intracellular ROS.

We observed a strong correlation between the degree of intracellular fluorescence, indicative of the extent of oxidative stress, with the capacity of the cells to perform their antitumoural effector functions (FIG. 4J). This correlation strongly suggests how activation of Nrf2, and lower levels of oxidative stress, directly leads to improved fitness, survival, and antitumoural activity of the NK cells.

We next investigated the same durations of contacting with activators of Nrf2 for TIL. We found that intracellular ROS levels in the melanoma patient derived TIL, KADA and BEHA, were in general higher than in NK cells, which explains why further increase by H₂O₂ treatment was difficult to detect by flow cytometry (data not shown). Nevertheless, for TIL, the fluorescence, which correlates to intracellular ROS levels, was lowered by AUF pre-treatment (FIG. 4K). In contrast to NK cells, the effects remained 72 h after AUF removal (FIG. 4K). In line with the results obtained for NK cells, the effector functions of AUF pre-treated TIL displayed an increased lysis of autologous tumour cells and for the highest AUF concentration, the effect lasted until 72 h after AUF withdrawal (FIGS. 4L-O).

These results show that AUF can improve NK cell and TIL tolerance towards oxidative stress for up to two to three days post pre-treatment. This is a clinically useful timeframe for adoptive cell therapy and cancer therapy and confers an advantage for the infused cells to persist in a ROS-rich TME and promoting the tumour elimination.

Improved Resistance Against Monocyte-Derived ROS with AUF Pre-Treated NK Cells

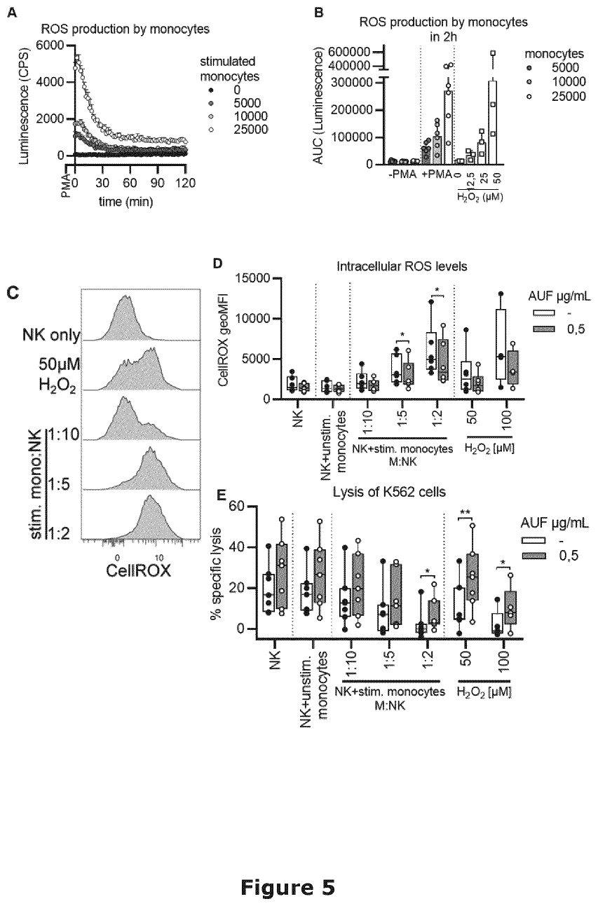

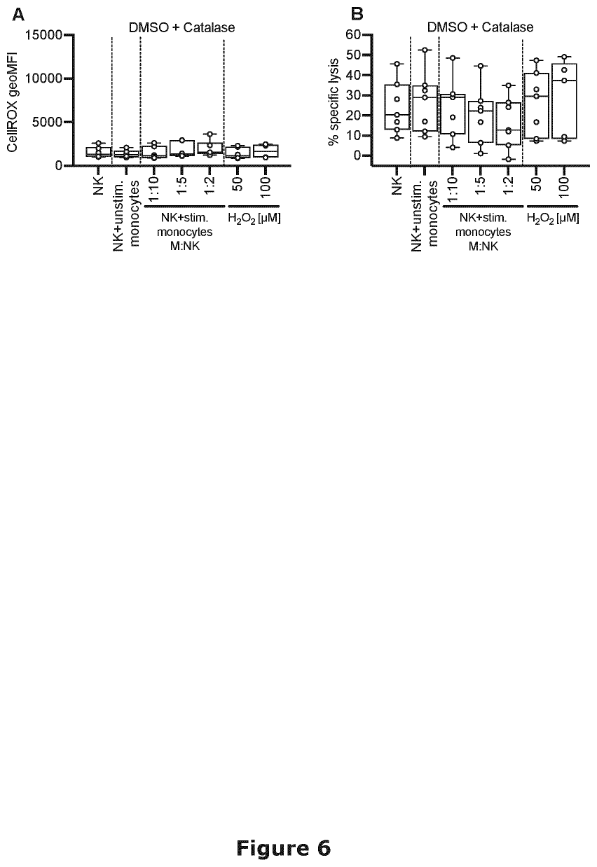

It has been shown that ROS, including hydrogen peroxide (H₂O₂), produced by autologous monocytes in the TME, leads to reduced NK- and T cell function. The inventors investigated if pre-treatment with AUF could increase NK cell and/or T cell resistance also towards activated monocytes derived ROS. Healthy donor derived monocytes were shortly activated with phorbol 12-myristate 13-acetate (PMA) and their production of ROS was subsequently investigated using luminescence. The levels of luminescence from ROS produced by the monocytes were compared with known H₂O₂ concentrations (FIGS. 5A-B). Using this experimental setup, the effect of co-culturing activated monocytes and autologous NK cells was investigated, as measured by assessment of the intracellular ROS levels and killing of K652 target cells. Increase in intracellular ROS in a dose (NK to monocyte ratio) dependent manner was observed in both untreated and AUF pre-treated NK cells (FIGS. 5C-D). However, the AUF pre-treated NK cells displayed significantly lower levels of intracellular ROS compared to controls (FIG. 5D). Furthermore, AUF pre-treated NK cells killed K562 target cells with a significantly higher efficiency in this setting, also when exposed to the highest monocyte to NK cell ratio (FIG. 5E). There was no difference in intracellular ROS or in the capacity to kill K562 tumour cells between the different conditions, without oxidative stress. There was no difference between activated monocytes or H₂O₂ treated control NK cells in the presence of catalase, indicating that monocyte derived suppression mainly is H₂O₂ mediated (FIGS. 6A-B). This shows that AUF pre-treatment NK cells could be of relevance ROS is produced by autologous immune suppressive cells.

Auranofin Pre-Treatment of NK Cells, CAR T Cells and TIL Improves their Antitumoural Efficacies Against Hematological and Solid Tumours.

To further investigate the potential of applying AUF pre-treatment in a clinical setting, we applied this treatment to ACT products used in the clinic. NK cells and CAR T cells have been demonstrated to show the highest efficiency against hematological cancer.

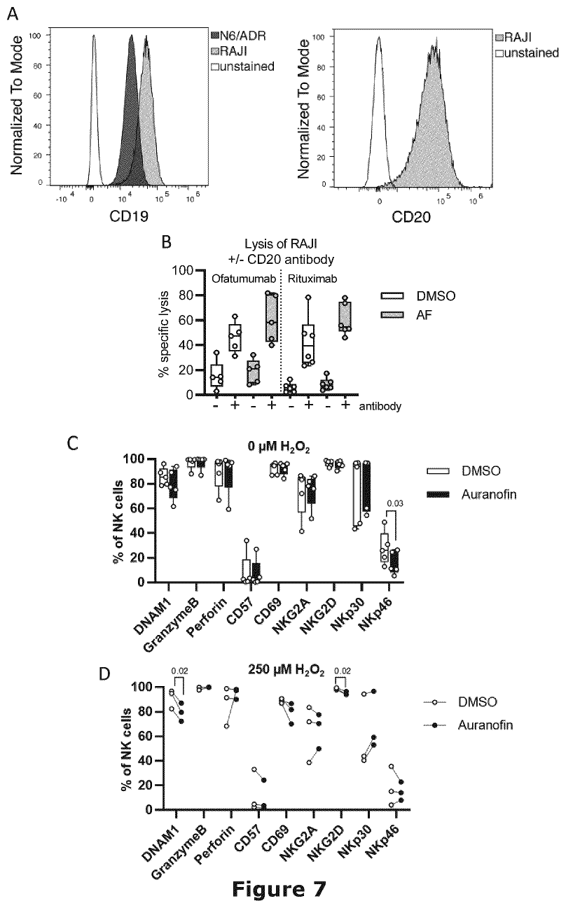

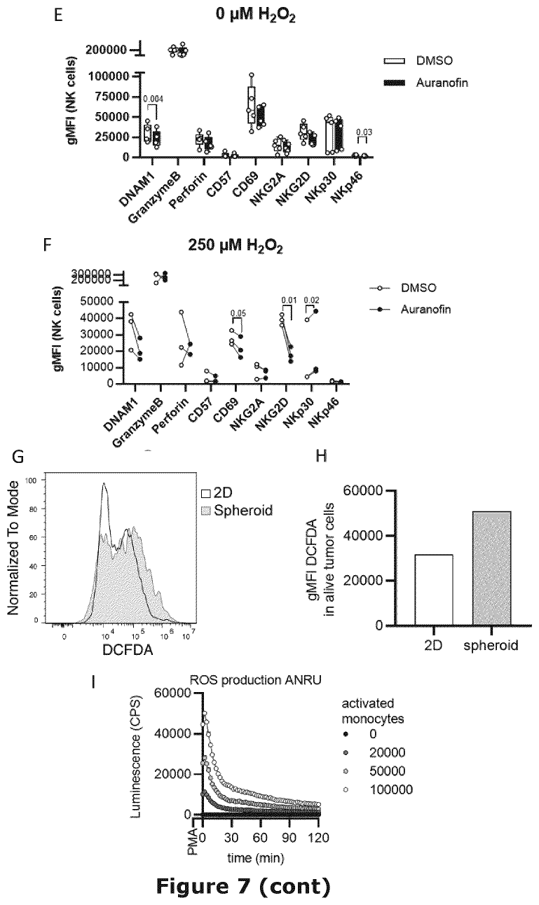

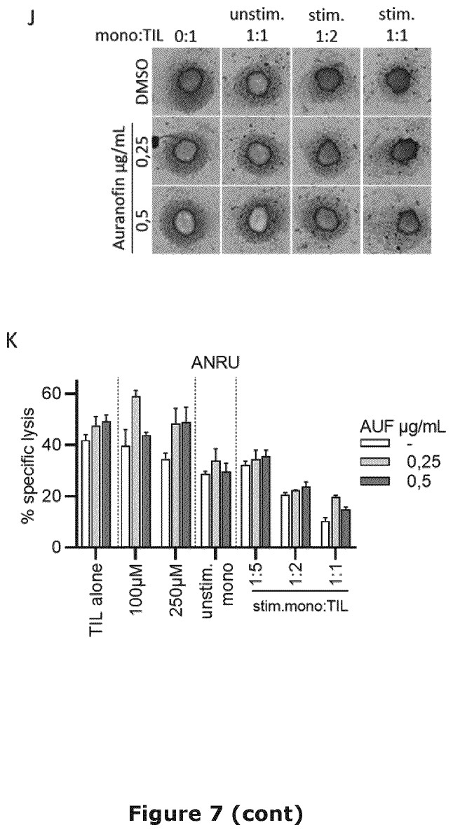

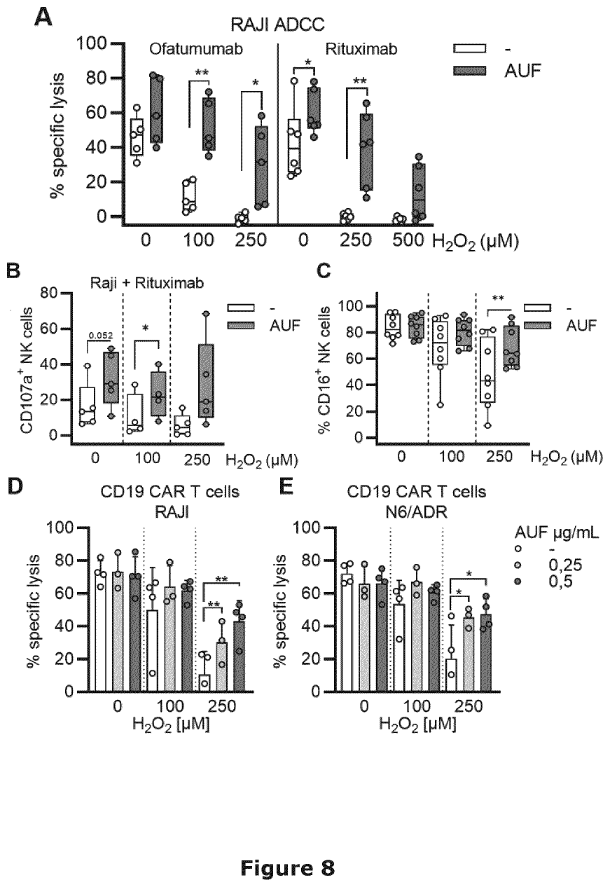

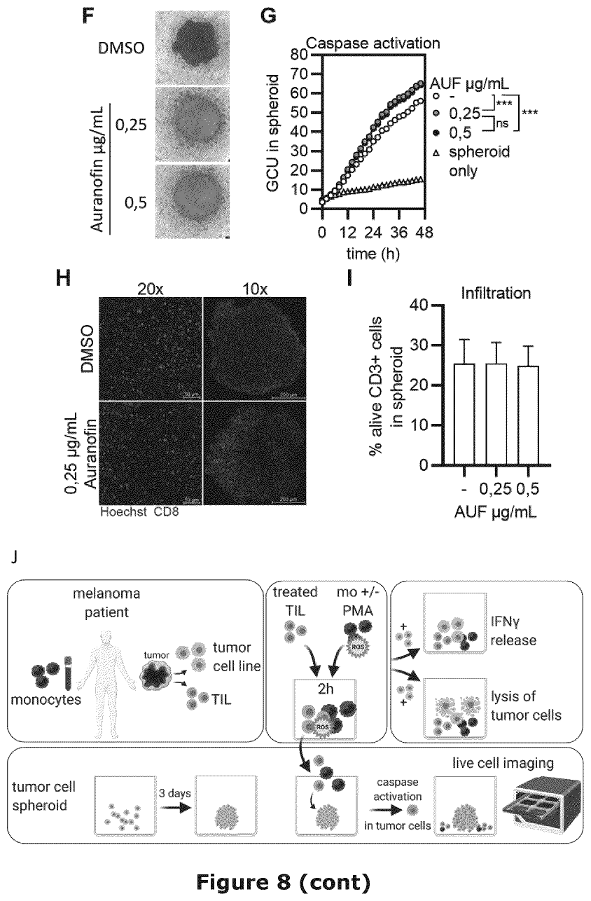

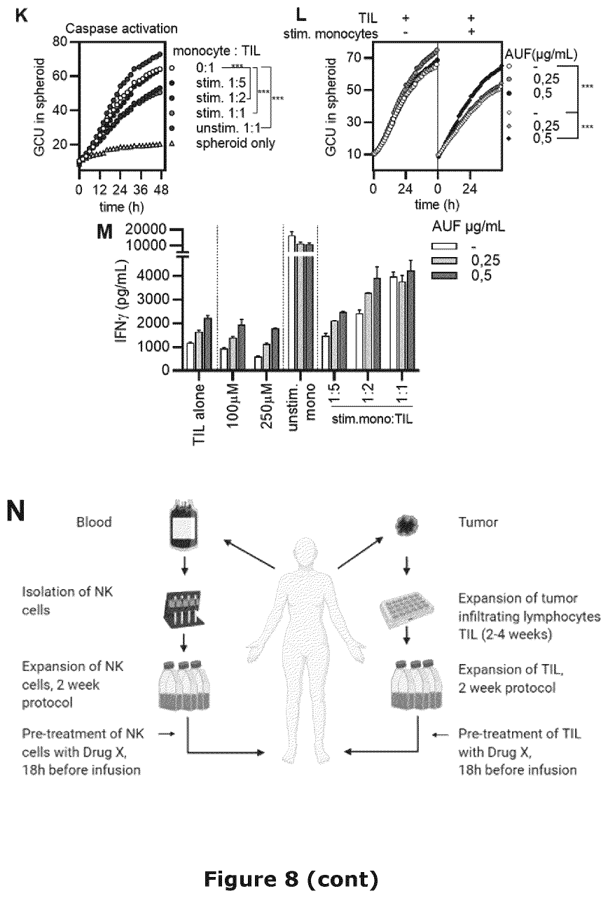

Patients with B cell malignancies are today typically treated with therapies targeting CD20 or CD19, commonly using antibody therapies targeting CD20, Rituximab and Ofatumumab, or CD19 directed CAR T cells. To this end, the effect of AUF pre-treated NK cells in combination with anti-CD20 therapy was assessed. RAJI cells, a CD20⁺CD19⁺ lymphoma cell line (FIG. 7A), were poorly recognized by NK cells (Suppl. FIG. 7B). However, when coated with either Rituximab or Ofatumumab, an efficient recognition of the target cells was observed, as detected by killing of the target cells and by degranulation (CD107a). The inventors found that AUF pre-treated NK cells displayed an increased capacity to recognize the anti-CD20 coated RAJI cells even without the presence of ROS (FIG. 8A). Upon addition of H₂O₂, a significant increase in the ability to lyse and degranulate in response to target cell recognition by the AUF pre-treated NK cells compared to control NK cells was observed (FIGS. 8A-B). A significant decrease in frequency of untreated NK cells expressing CD16 after exposure to H₂O₂ compared to AUF pre-treated NK cells was observed (FIG. 8C).

As mentioned above, the inventors found that NK cells displayed a weak recognize RAJI cells without addition of anti-CD20 (FIG. 7B). This emphasizes the importance of combining antibody mediated immunotherapy with an approach that alleviates the detrimental effect of factors such as ROS on the TME.

In addition to CD16, additional NK cell surface markers, DNAM-1, granzyme B, perforin, CD57, CD69, NKG2A, NKG2D, NKp40 and NKp46 were investigated after AUF pre-treatment with and without H₂O₂. Overall, few differences were observed between NK cells with and without AUF pre-treatment. Auranofin pre-treated NK cells displayed reduced frequency of positive NK cells and/or reduced cell surface levels of DNAM-1 and NKp46 or DNAM-1, CD69 and NKG2D, with and without exposure to H₂O₂, respectively (FIGS. 7C-F). However, upon exposure to H₂O₂ AUF pre-treated NK cells had an increased expression of NKp30.

The inventors investigated if AUF pre-treatment could improve the effects of CD19 directed CAR T cells in a ROS-rich environment. Either untreated or AUF pre-treated CD19+ CAR T cells were exposed to H₂O₂ at different concentrations and co-cultured with CD19+ lymphoma and leukemia target cells (RAJI and N6/ADR, respectively, FIG. 7A). AUF pre-treated CAR T cells showed significantly increased killing as compared to control CAR T cells. Thus, AUF pre-treatment can potentially improve already approved cancer therapies, such as antibody therapies, or CAR T cell ACT.

Neither NK cell nor CAR T cell therapies have yet shown to be efficient against solid tumours in a clinical setting. To model targeting solid tumours, the inventors carried out experiments to determine if AUF pre-treatment of TIL could increase their capacity to eliminate autologous tumour cells grown as spheroids (3D). Spheroids grown from tumour cells are 3D model system that mimics the solid tumour environment, including simulating an equivalent ROS environment. Therefore, the results are indicative of what will occur in vivo. Untreated or AUF pre-treated ANRU TIL were co-cultured with ANRU spheroids. A fluorescent green dye quantifying apoptosis via Caspase 3/7 activation was used to measure tumour elimination. In this model system AUF pre-treated ANRU TIL displayed a significantly increased capacity to induce apoptosis in the tumour cells compared to controls, as shown by increased green fluorescent intensity (FIGS. 8F-G). There was no difference in capacity to infiltrate the spheroid, as assessed by flow cytometry and confocal microscopy (FIGS. 8H-I). Thus, the increased capacity of AUF pre-treated TIL to eliminate the tumour cells was not due to increased infiltration, but rather due to improved effector functions. Tumour cells grown in 3D had higher intracellular ROS levels compared to conventionally grown tumour cells (FIGS. 7G-H), suggesting that the 3D structure induces increased oxidative stress. To further increase the levels of oxidative stress, untreated ANRU TIL were exposed to ROS released by activated autologous monocytes co-cultured at different ratios in regard to the TIL (FIGS. 8K and FIGS. 7I-K). TIL co-cultured with non-activated monocytes (unstim 1:1) had an increased capacity to eliminate the tumour spheroids compared to TIL cultured without addition of monocytes (0:1) (FIG. 8K). This may be expected, since monocytes are known to stimulate T cell activation. However, increasing number of activated monocytes per TIL caused a significant reduction of the TIL capacity to induce apoptosis in the spheroid grown tumour cells. Nevertheless, an increased caspase activity was observed in spheroids co-cultured with TIL pre-treated with the highest dose of AUF, compared to untreated control TIL. In addition, monocyte derived ROS impaired cytokine release by untreated TIL. This effect could partially be rescued by AUF pre-treatment (FIG. 5M).

Pre-treatment of NK cells, CAR T cells or TIL, with a compound activating Nrf2 significantly increases their resistance against oxidative stress and improves their general fitness and antitumoural activity. This treatment holds a great promise for improvement of cell products used for ACT to treat patients with hematological cancers or solid tumours.

### Material and Methods:

### Cell Lines and Cell Isolation:

Tumour and feeder cell line, K562, RAJI, N6/ADR, THP-1, KASUMI-1, KADA, ANRU, BEHA, and EBV-LCL feeder cell line (Lundqvist et al., 2011) were cultured in RPMI1640 or IMDM (both from Gibco) supplemented with 10-20% FBS (Gibco), penicillin (100 U/ml) and streptomycin (100 μg/ml) (both from LifeTechnologies). Patient derived melanoma cell lines, acronym ANRU, KADA and BEHA, were generated as previously described (Wickström et al., 2019). Cells grown in suspension, K562, RAJI, N6/ADR, KASUMI-1, THP-1, LCL, were culture at 0.5×10⁶ cells/mL, while adherent cells, KADA, ANRU, BEHA, were passaged every 2-5 days using 0.05% Trypsin-EDTA (Thermo Fisher Scientific).

Peripheral blood samples (anonymized by-products of blood donations from healthy adult donors) were purchased from Karolinska University Hospital Blood Bank. Peripheral blood mononuclear cells (PBMC) were isolated from healthy donor buffy coats using density centrifugation with Ficoll® Paque Plus (GE Healthcare). NK cells and CD14⁺ monocytes were isolated from PBMCs using NK cell isolation kit or CD14⁺ microbeads, respectively (both Miltenyi Biotec), following the manufacturer's instructions. For NK cell expansion, a EBV-LCL feeder cell line was used, irradiated at 100 gy and then co-cultured with NK cells, at the ratio 10:1 LCL:NK, in X-Vivo 20 (Lonza) supplemented with 10% human AB serum and 1000 U/mL IL-2 (Proleukin). From day 6 or 10, NK cells were kept at 0.5×10⁶ cells/mL or 1×10⁶/mL, respectively. Purity of expanded NK cell was assessed at day 10 by flow cytometry, see below.

Melanoma patient derived TIL and CD19 directed CAR T cells were generated as previously described (Magalhaes et al., 2018; Lövgren et al., 2020).

### Compounds and Oxidative Stress:

Lymphocytes were pre-treated for 18 h with Auranofin (AUF, with the concentration 1 μg/mL for NK cells and 0.5 μg/mL for TIL, if not indicated differently), DL-Sulforaphane (SUL) or Dimethyl Fumarate (DMF) (all Sigma-Aldrich) at indicated concentrations. Untreated/control NK cells were pre-treated with a DMSO concentration comparable to the highest concentration achieved by addition of the compounds.

For Nrf2 inhibition, NK cells were treated with 50 μM ML-385 (Sigma-Aldrich) in parallel. For H₂O₂ treatment, lymphocyte cells were washed and resuspended to 1×10⁶ cells/mL in medium and exposed to indicated H₂O₂ (Sigma-Aldrich) concentration for 1 h at 37° C. Cells were washed with medium and then used for further experiments. For monocyte experiments, autologous NK cell-monocytes or TIL-monocytes were used. NK cells/TIL were isolated and pre-treated with 0.5 μg/mL AUF, as described above, and co-cultured with unstimulated at the ratio, 1:2 M:NK, or stimulated monocytes at the ratio, 1:1 M:NK, if not indicated otherwise. Stimulated monocytes were activated with 100 ng/ml PMA for 1 min, washed and added at indicated ratio, the number of NK cell/TIL was kept constant. As control, H₂O₂ was used at indicated concentration. Cells were co-cultured for 2 h and then effector cells were stained for intracellular ROS (see flow cytometry) or used as effector cells in Cr⁵¹ release assay (E:T ratio 10:1).

### Detection of ROS Production by Monocytes:

Monocytes were stimulated with 100 ng/ml PMA for 1 min and washed with HBSS (Gibco). Cells were resuspended in HBSS 5% FBS and added to a 96-well Optiplate (Perkin Elmer) containing HBSS 5% FBS and Luminol (56 μM; Sigma). Luminescence was measured immediately using a EnSpire plate reader (Perkin Elmer).

### Flow Cytometry Staining:

All antibodies (see Table 1) and FACS reagents were used according to manufactures recommendation, if not stated otherwise. All antibodies had been titrated for optimal signal-to-noise ratio and all staining's were performed in PBS supplemented with 1% FBS. All staining's contained a live/dead marker. Samples were fixed with 2% PFA (Thermo scientific) for 15 min before acquired on a NovoCyte (ACEA Biosciences) and analysed using FlowJo Software (TreeStar).

TABLE 1
List of antibodies

| Specificity | Conjugation | Clone | Cat no. | Vendor |
|---|---|---|---|---|
| CD56 | PE-Cy7 | HCD56 | 318318 | BioLegend |
| CD3 | Pacific Blue | UCHT1 | 300431 | BioLegend |
| CD3 | PerCP | SK7 | 344808 | BioLegend |
| CD19 | FITC | SJ25C1 | 363008 | BioLegend |
| CD19 | BV570 | HIB19 | 302235 | BioLegend |
| CD20 | APC-Cy7 | 2H7 | 302313 | BioLegend |
| CD16 | Pacific Blue | 3G8 (RUO) | 558122 | BD Biosciences |
| CD107a | FITC | H4A3 | 328606 | BioLegend |
| CD8 | Unconjugated | C8/144B | 372902 | BioLegend |
| Mouse IgG | Alexa-Fluor | Poly4053 | 405322 | BioLegend |
| | 647 | | | |

For evaluating CD16 expression, NK cells were treated as described above. After H₂O₂ treatment, NK cells were incubated for 2 h in RPMI 2% AB serum, stained with anti-CD3, anti-CD56 and anti-CD16 and analyzed.

For detection of intracellular ROS levels, for NK cells CellROX™ Deep Red Reagent (Invitrogen) were used. Briefly, NK cells were stained with 2.5 μM CellROX solution in RPMI for 30 min at 37° C. and then stained for flow cytometry with anti-CD56 and anti-CD3 (both Biolegend).

For TIL, T cells were pre-incubated with 10 μM CM-H₂DCFDA (Thermo Fisher Scientific) solution in RPMI for 30 min at 37° C. prior to H₂O₂ treatment and then stained with anti-CD3, anti-CD4 and anti-CD8.

To study TIL infiltration into spheroids, eight spheroids of each condition were pooled and carefully washed twice with PBS, dissociated with trypsin and then stained with anti-CD3, CD4 and anti-CD8. The supernatant was collected to measure the non-infiltrated fraction.

Purity of expanded NK cell was determined at day 10 with flow cytometry, staining with anti-CD56 anti-CD3 and anti-CD19.

### Assessment of Effector Functions:

Lymphocytes were isolated and pre-treated when indicated, with compound and H₂O₂, as described above. For degranulation/measuring CD107a expression, NK cells were co-cultured with K562 tumour cells at an effector target ratio 1:1 in the presence of anti-CD107a in U-bottom plates. After 2 h, GolgiStop™ and GolgiPlug™ (BD Bioscience) were added and cells were harvested after an additional 4 h co-culture and stained for CD56 and CD3, as described above. For positive control, 25 ng/ml PMA (Sigma Aldrich) and 500 ng/mL Ionomycin (Sigma Aldrich) were added.

For cytotoxic assay, a standard 4 h [Cr⁵¹]-release assay was used. Briefly, tumour cells were harvested and labeled with 51Cr (PerkinElmer) and used as target cells. Effector cells, NK cells and T cells, were co-cultured in 96-well V-bottom plate with indicated tumour cells at the stated effector target ratio (E:T). The supernatant was collected onto LUMA plates (Perkin-Elmer) and radioactivity/tumour cell lysis was detected by MicroBeta2 (Perkin Elmer). Target cell killing measured by % specific lysis and calculated using the formula: ((experimental release-spontaneous release)/(maximum release-spontaneous release))*100.

For analyzing ADCC, NK cells were cultured with RAJI cells and Rituximab (0.5 μg/mL, MabThera, Roche) or Ofatumumab (0.05 μg/mL, Arzerra, Novartis).

For cytokine release assay, NK cells and TIL were cultured with indicated tumour cells at the effector target ratio 4:1 (E:T) for 24 h in U-bottom plates. IFNγ secretion was measured using human IFN-γ ELISA development kit (Mabtech) following the manufacturer's instructions.

### 3D Killing Assay:

5000 ANRU tumour cells were seeded per well in Ultra-Low Attachment 96-well plates (Corning Costar) in culture medium containing 2% Matrigel (Corning) for 3 days. 1×10⁴AUF pre-treated TIL (see above), and spheroids were co-cultured and monitored for 48 h with the IncuCyte live cell imaging system (Essen Bioscience) in the presence of CellEvent™ Caspase 3/7 Green detection agent (Invitrogen).

For confocal microscopy, spheroids were fixed with 4% PFA (Thermo Scientific). Spheroids stained with anti-CD8a followed by the secondary antibody (goat-anti-mouse IgG-AF647, Biolegend) and Hoechst 33342 dye (Invitrogen). Spheroids were cleared with 88% glycerol (Sigma) overnight, transferred to 8-well u-slides (Ibidi) and imaged with the Zeiss LSM800 confocal microscope. Spheroids were also analyzed for T cell infiltration using flow cytometry, see above.

### Evaluation of Nrf2 Target Gene Expression:

Lymphocytes were pre-treated with AUF, SUL or DMF as described above. RNA was isolated using TRIzol™ Plus RNA Purification Kit (Invitrogen). cDNA was generated using the iScript™ cDNA Synthesis Kit (Bio-Rad) and qPCR was done using iTaq™ Universal SYBR® Green Supermix (Bio-Rad) in the CFX96 Touch Real-Time PCR Detection System (Bio-Rad). Fold change expression from untreated cells was calculated using the 2\hat{ }-ΔΔCt formula with b-actin as reference gene. Evaluated genes were NAD(P)H Quinone Dehydrogenase 1 (NQO1), Kelch Like ECH Associated Protein 1 (Keap1), Heme Oxygenase 1 (HMOX1) and Thioredoxin Reductase 1 (TXNRD1).

TABLE 2
List of primer sequences (5′-3′):

| | |
|---|---|
| Keap1_for | TGCGTCCTGCACAACTGTATC |
| Keap1_rev | CCAGGAACGTGTGACCATCA |
| NQO1_for | CTGAAGGACCCTGCGAACT |
| NQO1_rev | TCGCTCAAACCAGCCTTTCAG |
| HMOX1_for | ACTCCCTGGAGATGACTCCC |
| HMOX1_rev | TCTTGCACTTTGTTGCTGGC |
| TXNRD1_for | ATATGGCAAGAAGGTGATGGTCC |
| TXNRD1_rev | GGGCTTGTCCTAACAAAGCTG |
| b-actin_for | CTCGCCTTTGCCGATCCG |
| b-actin_rev | TCTCCATGTCGTCCCAGTTG |

### Example 2

### Materials and Methods:

The materials and methods used in this Example are as set out for Example 1 and supplemented by the methods below.

Treatment with Compounds and Oxidative Stress

Lymphocytes were pre-treated for 18 h with Auranofin (AUF, with the concentration 1 μg/mL for NK cells and 0.5 μg/mL for TIL. As AUF was reconstituted in DMSO, control NK cells were pre-treated with DMSO with an equivalent volume to the highest compound concentration. For N-acetylcysteine (NAC, Invitrogen) treatment, DMSO pre-treated cells were incubated for 1 h with 5 or 10 mM NAC prior to H₂O₂ treatment. For H₂O₂ treatment, lymphocytes were washed and resuspended to 1×10⁶ cells/mL in RPMI containing the indicated H₂O₂ (Sigma-Aldrich) concentration for 1 h at 37° C. 5% CO₂. Cells were washed with medium and then used for further experiments.

### Flow Cytometry

All antibodies (see Table 1 in Example 1) and FACS reagents were used according to manufacturers' recommendation, if not stated otherwise. Cells were stained with LIVE/DEAD™ Fixable Aqua Dead Cell Stain Kit (Invitrogen) and then stained with the respective antibodies for 20 min at 4ºC in PBS 1% FBS. Intracellular staining was performed using Fixation/Permeabilization Kit (BD Biosciences) following the manufacturer's instructions. Samples without intracellular staining were fixed with 2% PFA (Thermo scientific) for 15 min before acquisition on a NovoCyte (ACEA Biosciences). All antibodies were titrated for optimal signal-to-noise ratio. Compensation was performed using AbC™ Total Antibody Compensation Bead Kit and ArC™ Amine Reactive Compensation Bead Kit (both Invitrogen). FlowJo Software (TreeStar) was used for analysis.

### Assessment of Effector Functions

Lymphocytes were isolated and pre-treated when indicated, with compounds and/or H₂O₂, as described above. For degranulation assay, NK cells were co-cultured with K562 cells (E:T ratio 1:1) in the presence of anti-CD107a FITC antibody. After 2 h, GolgiStop™ and GolgiPlug™ (BD Bioscience) were added and cells were harvested after an additional 4 h co-culture and stained for CD56, CD3 and anti-IFNγ, as described above. As a positive control, 25 ng/ml PMA (Sigma Aldrich) and 500 ng/ml Ionomycin (Sigma Aldrich) were added.

Targeting Intrinsic Antioxidant Pathways in NK Cells and TIL Preserves their Efficient Anti-Tumor Responses after H₂O₂ Exposure.

The inventors observed that AUF pre-treated NK cells have a better viability and function after exposure to oxidative stress/H₂O₂, similar to when adding catalase, an enzyme efficiently converting H₂O₂ to H₂O and O₂.

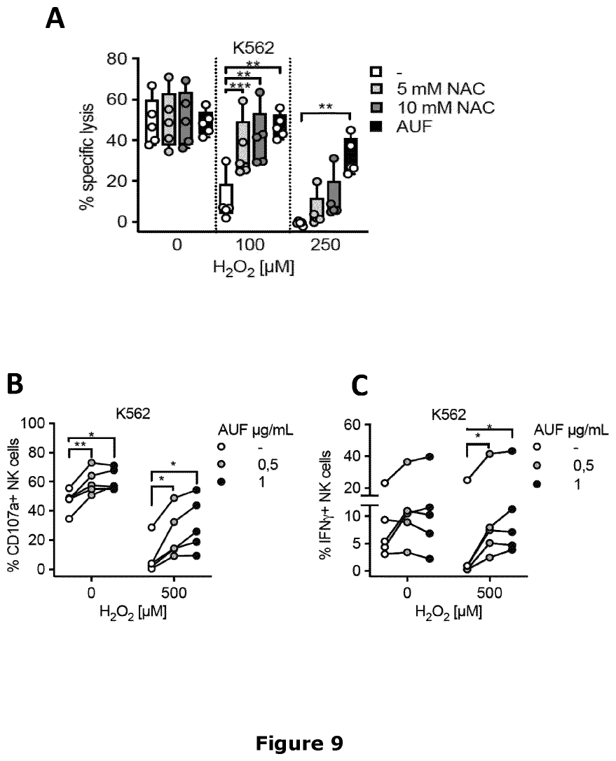

To further investigate and compare the protective effect of AUF pre-treatment, control NK cells were pre-treated for 1 h with N-acetylcysteine (NAC), a precursor to cysteine with a free thiol (SH) group increasing intracellular cysteine and glutathione levels proving several protective effects towards oxidative stress (1), resulted in a similar protection against exposure to 100 μM H₂O₂, but showed less protection against increased H₂O₂ concentrations compared to AUF (FIG. 9A). Thus, we concluded that AUF pre-treatment of NK cells provides a strong protective effect against ROS (H₂O₂).

We then investigated whether there was a difference in the protective effect mediated by AUF pre-treatment (e.g. does AUF pre-treatment protect different NK cell functions, degranulation/killing and cytokine production equally well). NK cells were pre-treated with AUF, exposed to H₂O₂ and evaluated for degranulation and cytokine production capacity after co-culture with K562 target cells, as measured by CD107a and IFNγ expression via flow cytometry (FIGS. 9B-C). There was no difference in AUF protective effect of different NK cell functions during oxidative stress. Notably, for CD107a the protective effect was observed both in the presence or absence of H₂O₂ (FIG. 9B). Note that FIG. 9B (Note that FIG. 9B corresponds to FIG. 1G and is presented again here as a reference).

### AUF Pre-Treatment of TIL do not Induce Regulatory T Cells.

It has previously been shown that indirectly activating Nrf2 by deleting or knocking down Keap1 in human and murine T cells, respectively, can also be used to trigger an increased expression of classical Nrf2 target genes (2, 3). However, CRISPR/cas9-mediated deletion of KEAP1 in human T cells seemed to preferentially work in CD4⁺ T cells, and increased the frequency CD4⁺ cells with a regulatory phenotype being less favorable for anti-cancer immunotherapy (2).

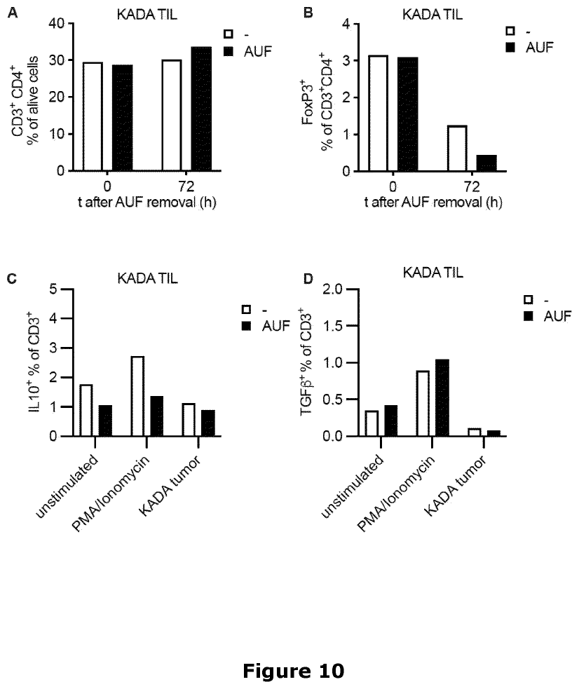

To further investigate this using AUF pre-treatment, KADA tumor cells were pre-treated with AUF and evaluated for alterations in frequency of CD4⁺ T cell and if the pre-treatment could induce regulatory T cells producing immunosuppressive cytokines. Notably, we did not observe any increase in frequency of CD4⁺ T cells after AUF pre-treatment and no induction of regulatory T cells could be detected at 0 h or 72 h post AUF pre-treatment of KADA TIL (FIG. 10).

### Example 2 References

1. G. Raghu, M. Berk, P. A. Campochiaro, H. Jaeschke, G. Marenzi, L. Richeldi, F.-Q. Wen, F. Nicoletti, P. M. A. Calverley, The Multifaceted Therapeutic Role of N-Acetylcysteine (NAC) in Disorders Characterized by Oxidative Stress. Current Neuropharmacology 19, 1202-1224 (2021).
2. S. Noel, S. A. Lee, M. Sadasivam, A. R. A. Hamad, H. Rabb, KEAP1 Editing Using CRISPR/Cas9 for Therapeutic NRF2 Activation in Primary Human T Lymphocytes. Journal of immunology (Baltimore, Md.: 1950) 200, 1929-1936 (2018).
3. S. Noel, M. N. Martina, S. Bandapalle, L. C. Racusen, H. R. Potteti, A. R. Hamad, S. P. Reddy, H. Rabb, T Lymphocyte-Specific Activation of Nrf2 Protects from AKI. J Am Soc Nephrol 26, 2989-3000 (2015).

### REFERENCES

Cebula, M., E. E. Schmidt, and E. S. Arner, “TrxR1 as a potent regulator of the Nrf2-Keap1 response system”, Antioxid Redox Signal, 2015. 23(10): p. 823-53.
Chakraborty, P., et al., “Thioredoxin-1 improves the immunometabolic phenotype of antitumor T cells”, J Biol Chem, 2019. 294(23): p. 9198-9212.
Chirullo, B., et al., “A candidate anti-HIV reservoir compound, auranofin, exerts a selective ‘anti-memory’ effect by exploiting the baseline oxidative status of lymphocytes”, Cell Death Dis, 2013. 4(12): e944.
Cummings and Schnellmann, “Measurement of Cell Death in Mammalian Cells”, Curr Protoc Pharmacol, 2004. Chapter 12, Unit 12.8.
Dikovskaya, D., et al., “Measuring the Interaction of Transcription Factor Nrf2 with Its Negative Regulator Keap1 in Single Live Cells by an Improved FRET/FLIM Analysis”, Chem Res Toxicol. 2019 Mar. 18; 32(3): 500-512.
Hansson, M., et al., “Induction of apoptosis in NK cells by monocyte-derived reactive oxygen metabolites”, J Immunol, 1996. 156(1): p. 42-7.
Harlin, H., et al., “The CD16-CD56 (bright) NK cell subset is resistant to reactive oxygen species produced by activated granulocytes and has higher antioxidative capacity than the CD16⁺CD56 (dim) subset”, J Immunol, 2007. 179 (7): p. 4513-9.
Hayakawa, Y., et al., “NK cell TRAIL eliminates immature dendritic cells in vivo and limits dendritic cell vaccination efficacy”, J Immunol, 2004. 172:123-129.
Hellstrand, K., “Histamine in cancer immunotherapy: a preclinical background”, Semin Oncol, 2002. 29 (3 Suppl 7): p. 35-40.
Kitteringham, N. R., et al., “Proteomic analysis of Nrf2 deficient transgenic mice reveals cellular defence and lipid metabolism as primary Nrf2-dependent pathways in the liver”, J Proteomics, 2010. 16; 73(8): 1612-31.
Kono, K., et al., “Hydrogen peroxide secreted by tumor-derived macrophages down-modulates signal-transducing zeta molecules and inhibits tumor-specific T cell- and natural killer cell-mediated cytotoxicity”, Eur J Immunol, 1996. 26(6): 1308-13.
Larsen, S. E., et al., “Differential cytokine withdrawal-induced death sensitivity of effector T cells derived from distinct human CD8⁺ memory subsets”, 2017. Cell Death Discov. 2017; 3: 17031.
Lei, X. G., et al., “Paradoxical Roles of Antioxidant Enzymes: Basic Mechanisms and Health Implications”, Physiol Rev, 2016. 96 (1): p. 307-64.
Lövgren, T., et al., Complete and long-lasting clinical responses in immune checkpoint inhibitor-resistant, metastasized melanoma treated with adoptive T cell transfer combined with DC vaccination. Oncoimmunology, 2020. 9 (1): p. 1792058.
Lundqvist, A., et al., “Bortezomib Treatment to Potentiate the Anti-tumor Immunity of Ex-vivo Expanded Adoptively Infused Autologous Natural Killer Cells”. J Cancer, 2011. 2:383-5.
Magalhaes, I., et al., CD19 Chimeric Antigen Receptor T Cells From Patients With Chronic Lymphocytic Leukemia Display an Elevated IFN-γ Production Profile. J Immunother, 2018. 41 (2): p. 73-83.
Mao, Y., et al., “IL-15 activates mTOR and primes stress-activated gene expression leading to prolonged antitumor capacity of NK cells”, Blood, 2016. 15; 128(11): 1475-89.
Mehrotra, S., et al., “Oxidative stress and lymphocyte persistence: implications in immunotherapy”, Adv Cancer Res, 2009. 102: p. 197-227.
Miller, C. G., et al., “NADPH-dependent and -independent disulfide reductase systems”, Free Radic Biol Med. 2018. 127: 248-261.
Multhoff, G. and Vaupel, P., “Hypoxia Compromises Anti-Cancer Immune Responses”, Adv Exp Med Biol. 2020. 1232: 131-143.
Norell, H., et al., “Inhibition of superoxide generation upon T-cell receptor engagement rescues Mart-1(27-35)-reactive T cells from activation-induced cell death”, Cancer Res, 2009. 69 (15): p. 6282-9.
Otsuji, M., et al., “Oxidative stress by tumor-derived macrophages suppresses the expression of CD3 zeta chain of T-cell receptor complex and antigen-specific T-cell responses”, Proc Natl Acad Sci USA, 1996. 93 (23): p. 13119-24.
Pietrobon, V. and Marincola, F. M., “Hypoxia and the phenomenon of immune exclusion”, J Transl Med. 2021; 19: 9.
Rigobello et al., “Inhibition of thioredoxin reductase by auranofin induces apoptosis in cisplatin-resistant human ovarian cancer cells”, Free Radic Biol Med. 2007 Mar. 15; 42(6): 872-81.
Robledinos-Anton, N., et al., “Activators and Inhibitors of NRF2: A Review of Their Potential for Clinical Development”, Oxid Med Cell Longev, 2019. 2019: 9372182.
Roder, C. and Thomson, M. J., “Auranofin: Repurposing an Old Drug for a Golden New Age”, Drugs R D. 2015. 15(1): 13-20.
Saei A. A. Comparative proteomics of dying and surviving cancer cells improves the identification of drug targets and sheds light on cell life/death decisions. Mol. Cell. Proteomics. 2018; 17(6): 1144-1155.
Schieber, M. and N. S. Chandel, “ROS function in redox signaling and oxidative stress”, Curr Biol, 2014. 24 (10): p. R453-62.
Schmielau, J. and O. J. Finn, “Activated granulocytes and granulocyte-derived hydrogen peroxide are the underlying mechanism of suppression of t-cell function in advanced cancer patients”, Cancer Res, 2001. 61 (12): p. 4756-60.
Sies, H. and Jones, D. P., “Reactive oxygen species (ROS) as pleiotropic physiological signalling agents”, Nature Reviews Molecular Cell Biology 2020, 21:363-383.
Stafford et al., “Irreversible inhibition of cytosolic thioredoxin reductase 1 as a mechanistic basis for anticancer therapy”, Sci Transl Med. 2018 Feb. 14; 10 (428): eaaf7444.
Terren, I., et al., “NK Cell Metabolism and Tumor Microenvironment”, Front Immunol. 2019. 24; 10:2278.
Vahlne G., et al., “In vivo tumor cell rejection induced by NK cell inhibitory receptor blockade: Maintained tolerance to normal cells even in the presence of IL-2”, Eur J Immunol, 2010. 40: 813-823.
Wickström, S. L., et al., “Cancer Neoepitopes for Immunotherapy: Discordance Between Tumor-Infiltrating T Cell Reactivity and Tumor MHC Peptidome Display”. Front Immunol, 2019. 10: p. 2766.
Yang et al. “Nrf2 Activators as Dietary Phytochemicals Against Oxidative Stress, Inflammation, and Mitochondrial Dysfunction in Autism Spectrum Disorders: A Systematic Review”, Front Psychiatry, 2020. 11:561998

### FURTHER EMBODIMENTS OF THE INVENTION

The present invention is also defined by reference to the embodiments in the numbered paragraphs below.
1. Use of an activator of Nrf2 for: preventing and/or reducing the suppression of NK cell and/or T cell activity caused by one or more stress; and/or inducing and/or maintaining and/or increasing NK cell and/or T cell activity in the presence of one or more stress; and/or increasing NK cell and/or T cell survival in response to one or more stress.
2. A method for: preventing and/or reducing the suppression of NK cell and/or T cell activity caused by one or more stress; and/or inducing and/or maintaining and/or increasing NK cell and/or T cell activity in the presence of one or more stress; and/or increasing NK cell and/or T cell survival in response to one or more stress;
   wherein the method comprises the step of contacting one or more NK cell and/or T cell with an activator of Nrf2.
3. The use or method according to any preceding paragraph, wherein the activator of Nrf2 is auranofin (triethylphosphine gold), dimethyl fumarate (DMF), sulforaphane, curcumin, resveratrol, naringenin and/or agmatine.
4. The use or method according to any preceding paragraph, wherein the one or more stress is present in a tumour microenvironment and/or is in the peripheral blood and/or organ of a cancer patient.
5. The use or method according to any preceding paragraph, wherein the one or more stress is oxidative stress, hypoxia, reoxygenation and/or starvation.
6. The use or method according to any preceding paragraph, wherein the increase in survival of the one or more NK cell and/or T cell and/or the effect on activity of the one or more NK cell and/or T cell is present in the absence of any known exogenous oxidative stress.
7. The use or method according to any preceding paragraph, wherein the one or more stress is oxidative stress.
8. The use or method according to any of paragraphs 5 to 7, wherein the oxidative stress comprises the presence of Reactive Oxygen Species (ROS).
9. The use or method according to paragraph 8, wherein the Reactive Oxygen Species (ROS), comprises one or more of hydrogen peroxide (H₂O₂), superoxide anions (O₂⁻), nitric oxide, hydroxyl radicals, hydroxyl ions and/or monocyte-derived ROS; and preferably wherein the Reactive Oxygen Species is hydrogen peroxide (H₂O₂).
10. The use or the method according to any preceding paragraph, wherein the NK cell and/or T cell activity is selected from one or more of:
   i) anti-cancer or anti-tumour activity;
   ii) production and/or release of cytokines;
   iii) production and/or release of IFN-γ;
   iv) effector function in tumour and/or spheroid tumour structures;
   v) specific lysis of a target cell, for example a tumour and/or a cancer cell;
   vi) degranulation and/or capacity to degranulate; and/or
   vii) ability to regulate and/or influence other immune cell types, such as Dendritic cells, macrophages or other monocyte/myeloid cell types, or other lymphocyte cell (e.g. NK mediated regulation of T cell activity and vice versa).
11. The use or the method according to any preceding paragraph, wherein the NK cell and/or T cell has an increased resistance to stress-induced cell death, for example oxidative stress-induced cell death, such as Reactive Oxygen Species-induced cell death and/or hydrogen peroxide-induced cell death, preferably where the increased resistance is relative to a NK cell and/or T cell that has not been contacted with and/or treated with and/or exposed to an activator of Nrf2, optionally wherein the stress results from treatment of a cancer patient with a therapeutic agent such as chemotherapy and/or radiation.
12. The use or the method according to any of the preceding paragraphs, wherein there is:
   (i) an increase of NK cell and/or T cell activity in the presence of the one or more stress;
   (ii) a reduction in suppression of NK cell and/or T cell activity caused by the one or more stress; and/or
   (iii) an increase in NK cell and/or T cell survival in the presence of the one or more stress;
   wherein the increase or reduction is in the range of 1 to 100%, such as at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 40%, 50%, 60%, 70%, 80%, 90%, 100% or greater.
13. The use or the method according to any of the preceding paragraphs, wherein there is:
   (i) an increase of NK cell and/or T cell activity in the presence of the one or more stress;
   (ii) a reduction in suppression of NK cell and/or T cell activity caused by the one or more stress; and/or
   (iii) an increase in NK cell and/or T cell survival in the presence of the one or more stress;
   wherein the increase or reduction is in the range of a 1.01-fold to 3-fold change, such as an increase of 1.01-fold, 1.02-fold, 1.03-fold, 1.04-fold, 1.05-fold, 1.06-fold, 1.07-fold, 1.09-fold, 1.1-fold, 1.2-fold, 1.25-fold, 1.3-fold, 1.4-fold, 1.5-fold, 1.6-fold, 1.7-fold, 1.8-fold, 1.9-fold, 2-fold, 2.25-fold, 2.5-fold, 3-fold or greater.
14. The use or the method according to any of paragraphs 12 or 13, wherein the increase or reduction is relative to one or more NK cell and/or T cell that has not been contacted with an activator of Nrf2.
15. The method according to any of paragraphs 2 to 14, comprising a step of obtaining one or more NK cell and/or T cell by apheresis (for example leukapheresis), by taking a blood sample (e.g. a peripheral blood sample or an umbilical cord blood sample), by taking a sample from an ascites, by draining one or more lymph node, or by biopsy (for example by biopsy of a tumour, optionally by biopsy of a solid tumour) and/or by venesection.
16. The method according to any of paragraphs 2 to 15, further comprising a step of administering the one or more NK cell and/or T cell to a patient in need thereof.
17. The method according to paragraph 16, wherein the patient in need thereof has cancer, optionally wherein the cancer is characterised by the presence of one or more stress, such as oxidative stress, hypoxia, reoxygenation, starvation.
18. The method according to any of paragraphs 16 or 17, wherein the patient in need thereof has been treated for cancer by an alternative therapeutic agent and/or method, and the risk of recurrence or progression of the cancer is reduced by administering the one or more NK cell and/or T cell to a patient.
19. The method according to any of paragraphs 2 to 18, wherein the step of contacting the NK cell and/or T cell with the activator of Nrf2 occurs ex vivo and/or in vitro.
20. An activator of Nrf2 for use in treating cancer in a patient, wherein the cancer is characterised by the presence of one or more stress, and wherein the treatment comprises the step of contacting the activator of Nrf2 with one or more NK cell and/or T cell.
21. Use of an activator of Nrf2 in the manufacture of a medicament for treating cancer in a patient, wherein the cancer is characterised by the presence of one or more stress, and wherein the treatment comprises the step of contacting the activator of Nrf2 with one or more NK cell and/or T cell.
22. A method for treating cancer in a patient, wherein the cancer is characterised by the presence of one or more stress, and wherein the method comprises the step of contacting one or more NK cell and/or T cell with an activator of Nrf2.
23. The activator of Nrf2 for use according to paragraph 20, the use according to paragraph 21, or the method according to paragraph 22, wherein the activator of Nrf2 prevents and/or reduces the suppression of NK cell and/or T cell activity caused by one or more stress; and/or induces and/or maintains and/or increases NK and/or T cell activity in the presence of one or more stress; and/or increases NK cell and/or T cell survival in response to one or more stress.
24. The activator of Nrf2 for use according to any of paragraphs 20 or 23, the use according to any of paragraphs 21 or 23, or the method according to the any of paragraphs 22 or 23, wherein the one or more stress is oxidative stress, hypoxia, reoxygenation and/or starvation, preferably wherein the one or more stress is oxidative stress.
25. The activator of Nrf2 for use according to any of paragraphs 20, 23 or 24, the use according to any of paragraphs 21, 23 or 24, or the method according to any of paragraphs 22 to 24, wherein the NK cell and/or T cell activity is selected from one or more of:
   i) anti-cancer or anti-tumour activity;
   ii) production and/or release of cytokines;
   iii) production and/or release of IFN-γ;
   iv) effector function in tumour and/or spheroid tumour structures;
   v) specific lysis of a target cell, for example a tumour and/or a cancer cell;
   vi) degranulation and/or capacity to degranulate; and/or
   vii) ability to regulate and/or influence other immune cell types, such as Dendritic cells, macrophages or other monocyte/myeloid cell types, or other lymphocyte cell (e.g. NK mediated regulation of T cell activity and vice versa).
26. The activator of Nrf2 for use according to any of paragraphs 20 or 23 to 25, the use according to any of paragraphs 21 or 23 to 25, or the method according to any of paragraphs 22 to 25, wherein the NK cell and/or T cell has an increased resistance to stress-induced cell death, for example reactive oxygen species-induced cell death, such as hydrogen peroxide-induced cell death, preferably where the increased resistance is relative to a NK cell and/or T cell that has not been contacted with and/or treated with and/or exposed to an activator of Nrf2.
27. The activator of Nrf2 for use according to any of paragraphs 20 or 23 to 26, the use according to any of paragraphs 21 or 23 to 26, or the method according to any of paragraphs 22 to 26, wherein the activator of Nrf2 is auranofin (triethylphosphine gold), dimethyl fumarate (DMF), sulforaphane, curcumin, resveratrol, naringenin and/or agmatine.
28. The activator of Nrf2 for use according to any of paragraphs 20 or 23 to 27, the use according to any of paragraphs 21 or 23 to 27, or the method according to any of paragraphs 22 to 27, wherein the one or more stress is oxidative stress comprising the presence of Reactive Oxygen Species, optionally wherein the Reactive Oxygen Species, comprises one or more of hydrogen peroxide (H₂O₂), superoxide anions (O₂⁻) nitric oxide, hydroxyl radicals, hydroxyl ions and/or monocyte-derived ROS; preferably wherein the Reactive Oxygen Species is hydrogen peroxide (H₂O₂).
29. The activator of Nrf2 for use according to any of paragraphs 20 or 23 to 28, the use according to any of paragraphs 21 or 23 to 28, or the method according to any of paragraphs 22 to 28, wherein the NK cell and/or T cell is contacted with the activator of Nrf2 ex vivo and/or in vitro.
30. The activator of Nrf2 for use according to any of paragraphs 20 or 23 to 29, the use according to any of paragraphs 1, 3 to 14, 21 or 23 to 29, or the method according to any of paragraphs 2 to 19 or 22 to 29, wherein the one or more NK cell and/or T cell is autologous.
31. The activator of Nrf2 for use according to any of paragraphs 20 or 23 to 30, the use according to any of paragraphs 1, 3 to 14, 21 or 23 to 30, or the method according to any of paragraphs 2 to 19 or 22 to 30, wherein the one or more NK cell and/or T cell is obtained from the patient.
32. The activator of Nrf2 for use according to any of paragraphs 20 or 23 to 31, the use according to any of paragraphs 1, 3 to 14, 21 or 23 to 31, or the method according to any of paragraphs 2 to 19 or 22 to 31, wherein the one or more NK cell and/or T cell is allogenic.
33. The activator of Nrf2 for use according to any of paragraphs 20 or 23 to 32, the use according to any of paragraphs 1, 3 to 14, 21 or 23 to 32, or the method according to any of paragraphs 2 to 19 or 22 to 32, wherein the one or more NK cell and/or T cell is obtained from a donor.
34. The activator of Nrf2 for use according to any of paragraphs 20 or 23 to 33, the use according to any of paragraphs 21 or 23 to 32, or the method according to any of paragraphs 15 to 19 or 22 to 33, wherein the treatment or method comprises a step of obtaining one or more NK cell and/or T cell from the patient or from a donor.
35. The activator of Nrf2 for use, the use, or the method according to paragraph 34, wherein the one or more NK cell and/or T cell is obtained by apheresis (for example leukapheresis), by taking a blood sample (e.g. a peripheral blood sample or an umbilical cord blood sample), by taking a sample from an ascites, by draining one or more lymph node, or by a biopsy (for example by a biopsy of a tumour, optionally by a biopsy of a solid tumour) and/or by venesection.
36. The activator of Nrf2 for use according to any of paragraphs 20 or 23 to 35, the use according to any of paragraphs 1, 3 to 14, 21 or 23 to 35, or the method according to any of paragraphs 2 to 19 or 22 to 35, wherein the one or more NK cell and/or T cell is contacted with an activator of Nrf2 for up to 48 hours, optionally between 0.5 and 24 hours, and preferably for 0.1 hours, 0.2 hours, 0.3 hours, 0.4 hours, 0.5 hours, 0.75 hours, 1 hour, 2 hours, 3 hours, 4 hours, 5 hours, 6 hours, 7 hours, 8 hours, 9 hours, 10 hours, 11 hours, 12 hours, 14 hours, 16 hours, 18 hours or 24 hours.
37. The activator of Nrf2 for use according to any of paragraphs 20 or 23 to 36, the use according to any of paragraphs 1, 3 to 14, 21 or 23 to 36, or the method according to any of paragraphs 2 to 19 or 22 to 36, further comprising an optional post-contacting step, wherein the one or more NK cell and/or T cell are incubated in the absence of the activator of Nrf2; optionally wherein the activator of Nrf2 is washed away from the one or more NK cell and/or T cell.
38. The activator of Nrf2 for use, the use, or the method according to paragraph 37, wherein the post-contacting step is performed for up to 48 hours, optionally for between 0.5 hours to 24 hours, preferably for 0.1 hours, 0.2 hours, 0.3 hours, 0.4 hours, 0.5 hours, 0.75 hours, 1 hour, 2 hours, 3 hours, 4 hours, 5 hours, 6 hours, 7 hours, 8 hours, 9 hours, 10 hours, 11 hours, 12 hours, 14 hours, 16 hours, 17.5 hours, 18 hours or 24 hours.
39. The activator of Nrf2 for use, the use, or the method according to any of paragraphs 36 to 38, wherein the total duration of the contacting step and/or post-contacting step is at least 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 20 hours, 22 hours, 24 hours, 48 hours, 72 hours or greater; optionally wherein the total duration of the contacting step and/or post-contacting step is between 4 and 24 hours, and preferably between 6 and 18 hours.
40. The activator of Nrf2 for use according to any of paragraphs 20 or 23 to 39, the use according to any of paragraphs 21 or 23 to 39, or the method according to any of paragraphs 16 to 19 or 22 to 39, wherein the method or treatment further comprises a step of administering the cells to the patient, optionally after the contacting step and/or a post-contacting step.
41. The activator of Nrf2 for use according to any of paragraphs 20 or 23 to 40, the use according to any of paragraphs 21 or 23 to 40, or the method according to any of paragraphs 16 to 19 or 22 to 40, wherein the treatment or method comprises a step of administering an effective amount of the one or more NK cell and/or T cell to the patient, optionally after the contacting step and/or a post-contacting step.
42. The activator of Nrf2 for use according to paragraph 40 or 41, the use according to paragraph 40 or 41 and/or the method according to any of paragraphs 16 to 19, 40 or 41, wherein the one or more NK cell and/or T cell is administered by infusion and/or injection and/or intravenously.
43. The activator of Nrf2 for use according to any of paragraphs 20 or 23 to 42, the use according to any of paragraphs 21 or 23 to 42, or the method according to any of paragraphs 16 to 19 or 22 to 42, wherein the activator of Nrf2 is removed and/or washed away before an effective amount of the one or more NK cell and/or T cell is administered to the patient.
44. The activator of Nrf2 for use according to any of paragraphs 20 or 23 to 43, the use according to any of paragraphs 1, 3 to 14, 21 or 23 to 43, or the method according to any of paragraphs 2 to 19 or 22 to 43, wherein the effect on the one or more NK cell and/or T cell activity and/or survival is present for sufficient time to allow for the treatment and/or prevention of cancer.
45. The activator of Nrf2 for use according to any of paragraphs 20 or 23 to 44, the use according to any of paragraphs 1, 3 to 14, 21 or 23 to 44, or the method according to any of paragraphs 2 to 19 or 22 to 44, wherein the effect on NK cell and/or T cell activity and/or survival is present for at least 24 hours, at least 30 hours, at least 36 hours, at least 42 hours, at least 48 hours, at least 54 hours, at least 60 hours, at least 66 hours, and/or at least 72 hours after the contacting step and/or the post-contacting step and/or after the activator of Nrf2 is removed and/or washed away from the one or more NK cell and/or T cell.
46. The activator of Nrf2 for use according to any of paragraphs 20 or 23 to 45, the use according to any of paragraphs 1, 3 to 14, 21 or 23 to 45, or the method according to any of paragraphs 2 to 19 or 22 to 45, wherein the effect on NK cell and/or T cell activity and/or survival is reversible and/or is no longer present at least 72 hours, at least 84 hours or at least 96 hours after the contacting step and/or the post-contacting step and/or after the activator of Nrf2 is removed and/or washed away from the one or more NK cell and/or T cell.
47. The activator of Nrf2 for use according to any of paragraphs 20 or 23 to 46, the use according to any of paragraphs 21 or 23 to 46, or the method according to any of paragraphs 2 to 19 or 22 to 46, wherein the cancer is a bladder cancer, bone cancer, breast cancer, colon cancer, cervical cancer, rectal cancer, endometrial cancer, oesophageal cancer, ovarian cancer, gastric cancer, kidney cancer, leukaemia, liver cancer, lung cancer, skin cancer (including basal cell carcinoma, squamous cell carcinoma, melanoma, non-skin located uveal melanoma and/or mucosal melanoma), lymphoma, pancreatic cancer, prostate cancer, testicular cancer and/or thyroid cancer.
48. The activator of Nrf2 for use according to any of paragraphs 20 or 23 to 47, the use according to any of paragraphs 21 or 23 to 47, or the method according to any of paragraphs 2 to 19 or 23 to 47, wherein the cancer is a solid tumour, optionally wherein there is a high level of one or more stress, such as oxidative stress, present in the solid tumour microenvironment.
49. The activator of Nrf2 for use according to any of paragraphs 20 or 23 to 48, the use according to any of paragraphs 1, 3 to 14, 21 or 23 to 48 or the method according to any of paragraphs 2 to 19 or 22 to 48, wherein the NK cell and/or T cell is contacted with an activator of Nrf2 at a concentration between 0.1 μM and 500 μM, for example wherein the concentration of the activator of Nrf2 is 0.1 μM, 0.25 μM, 0.5 μM, 0.75 μM, 1 μM, 1.5 M, 2 μM, 2.5 M, 3 μM, 4 μM, 5 μM, 7.5 μM, 10 μM, 15 μM, 20 μM, 25 μM, 30 μM, 40 μM, 45 μM, 50 μM, 60 μM, 70 M, 80 M, 90 μM, 100 μM, 150 μM, 200 μM, 250 μM, 300 μM, 350 μM, 400 μM, 450 μM or 500 μM.
50. The activator of Nrf2 for use according to any of paragraphs 20 or 23 to 49, the use according to any of paragraphs 1, 3 to 14, 21, or 23 to 49 or the method according to any of paragraphs 2 to 19 or 22 to 49, wherein the NK cell and/or T cell is contacted with an activator of Nrf2 at a concentration between 0.1 μg/ml and 500 μg/ml, for example wherein the concentration of the activator of Nrf2 is 0.1 μg/ml, 0.25 μg/ml, 0.5 μg/ml, 0.75 μg/ml, 1 μg/ml, 1.5 μg/ml, 2 μg/ml, 2.5 μg/ml, 3 μg/ml, 4 μg/ml, 5 μg/ml, 7.5 μg/ml, 10 μg/ml, 15 μg/ml, 20 μg/ml, 25 μg/ml, 30 μg/ml, 40 μg/ml, 45 μg/ml, 50 μg/ml, 60 μg/ml, 70 μg/ml, 80 μg/ml, 90 μg/ml, 100 μg/ml, 150 μg/ml, 200 μg/ml, 250 μg/ml, 300 μg/ml, 350 μg/ml, 400 μg/ml, 450 μg/ml or 500 μg/ml.
51. The activator of Nrf2 for use according to any of paragraphs 20 or 23 to 50, the use according to any of paragraphs 1, 3 to 14, 21 or 23 to 50 or the method according to any of paragraphs 2 to 19 or 22 to 50, wherein the activator of Nrf2 is auranofin and wherein the NK cell and/or T cell is contacted with auranofin at a concentration of 1 μM, 5 μM, 10 μM or 25 μM, 0.25 μg/ml, 0.5 μg/ml or 1 μg/ml.
52. The activator of Nrf2 for use according to any of paragraphs 20 or 23 to 50, the use according to any of paragraphs 1, 3 to 14, 21 or 23 to 50 or the method according to any of paragraphs 2 to 19 or 22 to 50, wherein the activator of Nrf2 is sulforaphane and wherein the NK cell and/or T cell is contacted with sulforaphane at a concentration of 2.5 μM, 5 M, 10 μM or 25 μM.
53. The activator of Nrf2 for use according to any of paragraphs 20 or 23 to 50, the use according to any of paragraphs 1, 3 to 14, 21 or 23 to 50 or the method according to any of paragraphs 2 to 19 or 22 to 50, wherein the activator of Nrf2 is dimethyl fumarate (DMF) and wherein the NK cell and/or T cell is contacted with dimethyl fumarate (DMF) at a concentration of 5 μM, 10 μM or 25 μM.
54. The activator of Nrf2 for use according to any of paragraphs 20 or 23 to 53, the use according to any of paragraphs 1, 3 to 14, 21 or 23 to 53 or the method according to any of paragraphs 2 to 19 or 22 to 53, wherein the number of one or more NK cell and/or T cells present is between 1×10⁴ and 1×10⁸ cells, for example 1×10⁴, 1×10⁵, 1×10⁶, 1×10⁷, 1×10⁸ cells, preferably 1×10⁶ cells.
55. The activator of Nrf2 for use according to any of paragraphs 20 or 23 to 54, the use according to any of paragraphs 1, 3 to 14, 21 or 23 to 54 or the method according to any of paragraphs 2 to 19 or 22 to 54, wherein the one or more T cell is a tumour infiltrating lymphocyte (TIL) and/or a chimeric antigen receptor T cell (CAR-T cell).
56. The activator of Nrf2 for use according to any of paragraphs 40 to 55, the use according to any of paragraphs 40 to 55 or the method according to any of paragraphs 16 to 19 or 40 to 55, wherein the one or more NK cell and/or T cell is administered in combination with one or more further therapeutic agent, particularly wherein the one or more further therapeutic agent is an anti-cancer therapeutic agent, such as an anti-cancer antibody, an anti-CD20 agent (e.g. Ofatumumab or Rituximab), a chimeric antigen receptor T cell (e.g. a CD19+ chimeric antigen receptor T cell) and/or non-activated monocytes.
57. A method for inducing and/or increasing NK cell and/or T cell activity wherein the method comprises a step of contacting one or more NK cell and/or T cell with an activator of Nrf2 ex vivo, wherein the method further comprises a step of administering the one or more NK cell and/or T cell to a patient in need thereof.
58. The method according to paragraph 57 wherein the patient in need thereof has cancer, optionally wherein the cancer is characterised by the presence of one or more stress, such as oxidative stress, hypoxia, reoxygenation, starvation.
59. The method according to any of paragraphs 57 or 58 wherein the NK cell and/or T cell activity is selected from one or more of:
   i) anti-cancer or anti-tumour activity;
   ii) production and/or release of cytokines;
   iii) production and/or release of IFN-γ;
   iv) effector function in tumour and/or spheroid tumour structures;
   v) specific lysis of a target cell, for example a tumour and/or a cancer cell; and/or
   vi) degranulation and/or capacity to degranulate;
   vii) ability to regulate and/or influence other immune cell types, such as Dendritic cells, macrophages or other monocyte/myeloid cell types, or other lymphocyte cell (e.g. NK mediated regulation of T cell activity and vice versa).
60. The method according to any of paragraphs 57 to 59, wherein the one or more NK cell and/or T cell is contacted with an activator of Nrf2 for up to 48 hours, optionally between 0.5 and 24 hours, and preferably for 0.1 hours, 0.2 hours, 0.3 hours, 0.4 hours, 0.5 hours, 0.75 hours, 1 hour, 2 hours, 3 hours, 4 hours, 5 hours, 6 hours, 7 hours, 8 hours, 9 hours, 10 hours, 11 hours, 12 hours, 14 hours, 16 hours, 18 hours or 24 hours.
61. The method according to any of paragraphs 57 to 60 wherein the effect on NK cell and/or T cell activity is present for at least 24 hours, at least 30 hours, at least 36 hours, at least 42 hours, at least 48 hours, at least 54 hours, at least
60 hours, at least 66 hours, and/or at least 72 hours after the contacting step and/or after the activator of Nrf2 is removed and/or washed away from the one or more NK cell and/or T cell.
62. A use, method or activator of Nrf2 substantially as described herein with reference to the accompanying claims, paragraphs, description and/or figures.

1. (canceled)

2. A method for: increasing and/or maintaining and/or inducing T cell and/or NK cell activity in the presence of one or more stress; and/or reducing and/or preventing the suppression of T cell and/or NK cell activity caused by one or more stress; and/or increasing T cell and/or NK cell survival in response to one or more stress;
wherein the method comprises the step of contacting one or more T cell and/or NK cell with an activator of Nrf2.

3. The method according to claim 2, for increasing T cell and/or NK cell activity in the presence of one or more stress.

4. The method according to claim 2, wherein the activator of Nrf2 is auranofin (triethylphosphine gold), dimethyl fumarate (DMF), sulforaphane, curcumin, resveratrol, naringenin and/or agmatine.

5. The method according to claim 2, wherein the one or more stress is present in a tumour microenvironment and/or is in the peripheral blood and/or organ of a cancer patient.

6. The method according to claim 2, wherein the one or more stress is oxidative stress, hypoxia, reoxygenation and/or starvation.

7. The method according to claim 2, wherein the increase in survival of the one or more T cell and/or NK cell and/or the effect on activity of the one or more T cell and/or NK cell is present in the absence of any known exogenous oxidative stress.

8. The method according to claim 2, wherein the one or more stress is oxidative stress.

9-10. (canceled)

11. The method according to claim 2, wherein
the NK cell and/or T cell activity is selected from one or more of:
i) anti-cancer or anti-tumour activity;
ii) production and/or release of cytokines;
iii) production and/or release of IFN-γ;
iv) effector function in tumour and/or spheroid tumour structures;
v) specific lysis of a target cell, for example a tumour and/or a cancer cell;
vi) degranulation and/or capacity to degranulate; and/or
vii) ability to regulate and/or influence other immune cell types, such as Dendritic cells, macrophages or other monocyte/myeloid cell types, or other lymphocyte cell (e.g. NK mediated regulation of T cell activity and vice versa).

12. The method according to claim 2, wherein the T cell and/or NK cell has an increased resistance to stress-induced cell death, for example oxidative stress-induced cell death, such as Reactive Oxygen Species-induced cell death and/or hydrogen peroxide-induced cell death, preferably where the increased resistance is relative to a T cell and/or NK cell that has not been contacted with and/or treated with and/or exposed to an activator of Nrf2, optionally wherein the stress results from treatment of a cancer patient with a therapeutic agent such as chemotherapy and/or radiation.

13. The method according to claim 2, wherein there is:
(i) an increase of T cell and/or NK cell activity in the presence of the one or more stress;
(ii) a reduction in suppression of T cell and/or NK cell activity caused by the one or more stress; and/or
(iii) an increase in T cell and/or NK cell survival in the presence of the one or more stress;
wherein the increase or reduction is in the range of 1 to 100%, such as at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 40%, 50%, 60%, 70%, 80%, 90%, 100% or greater.

14. The method according to claim 2, wherein there is:
(i) an increase of T cell and/or NK cell activity in the presence of the one or more stress;
(ii) a reduction in suppression of T cell and/or NK cell activity caused by the one or more stress; and/or
(iii) an increase in T cell and/or NK cell survival in the presence of the one or more stress; wherein the increase or reduction is in the range of a 1.01-fold to 3-fold change, such as an increase of 1.01-fold, 1.02-fold, 1.03-fold, 1.04-fold, 1.05-fold, 1.06-fold, 1.07-fold, 1.09-fold, 1.1-fold, 1.2-fold, 1.25-fold, 1.3-fold, 1.4-fold, 1.5-fold, 1.6-fold, 1.7-fold, 1.8-fold, 1.9-fold, 2-fold, 2.25-fold, 2.5-fold, 3-fold or greater.

15. (canceled)

16. The method according to claim 2, comprising a step of obtaining one or more T cell and/or NK cell by apheresis (for example leukapheresis), by taking a blood sample (e.g. a peripheral blood sample or an umbilical cord blood sample), by taking a sample from an ascites, by draining one or more lymph node, or by biopsy (for example by biopsy of a tumour, optionally by biopsy of a solid tumour) and/or by venesection.

17. The method according to claim 2, further comprising a step of administering the one or more T cell and/or NK cell to a patient in need thereof.

18. The method according to claim 17, wherein the patient in need thereof has cancer, optionally wherein the cancer is characterised by the presence of one or more stress, such as oxidative stress, hypoxia, reoxygenation, starvation.

19. (canceled)

20. The method according to claim 2, wherein the step of contacting the T cell and/or NK cell with the activator of Nrf2 occurs ex vivo and/or in vitro.

21-22. (canceled)

23. A method for treating cancer in a patient, wherein the cancer is characterised by the presence of one or more stress, and wherein the method comprises the step of contacting one or more T cell and/or NK cell with an activator of Nrf2.

24-30. (canceled)

31. The method according to claim 2, wherein the one or more T cell and/or NK cell is autologous.

32. The method according to claim 2, wherein the one or more T cell and/or NK cell is obtained from the patient.

33. The method according to claim 2, wherein the one or more T cell and/or NK cell is allogenic.

34. The method according to claim 2, wherein the one or more T cell and/or NK cell is obtained from a donor.

35. The method according to any of claim 16, wherein the treatment or method comprises a step of obtaining one or more T cell and/or NK cell from the patient or from a donor.

36. (canceled)

37. The method according to any of claim 16, wherein the T cell is a tumor infiltrating lymphocyte (TIL).

38. The method according to claim 2, wherein the method is a method for promoting regression of a cancer in a mammal by expanding tumor infiltrating lymphocytes (TILs) into a therapeutic population of TILs comprising:
(a) culturing autologous T cells by obtaining a first population of TILs from a tumor resected from a mammal,
(b) performing a first expansion by culturing the first population of TILs in a cell culture medium comprising IL-2;
(c) performing a second expansion by supplementing the cell culture medium of the second population of TILs with additional IL-2, anti-CD3 antibodies, and antigen presenting cells (APCs), to produce a third population of TILs, wherein the third population of TILs is a therapeutic population;
(d) reducing and/or preventing the suppression of T cell, such as TIL, activity caused by one or more stress; and/or inducing and/or maintaining and/or increasing T cell activity in the presence of one or more stress; and/or increasing T cell survival in response to one or more stress by contacting the third population of TILs with an activator of Nrf2, and
(e) after administering nonmyeloablative lymphodepleting chemotherapy, administering to the mammal the therapeutic population of T cells, wherein the T cells administered to the mammal, whereupon the regression of the cancer in the mammal is promoted.

39. The method according to claim 2, wherein the method is a method for treating a subject with cancer comprising administering expanded tumor infiltrating lymphocytes (TILs) comprising:
(a) culturing autologous T cells by obtaining a first population of TILs from a tumor resected from a mammal,
(b) performing a first expansion by culturing the first population of TILs in a cell culture medium comprising IL-2;
(c) performing a second expansion by supplementing the cell culture medium of the second population of TILs with additional IL-2, anti-CD3 antibodies, and antigen presenting cells (APCs), to produce a third population of TILs, wherein the third population of TILs is a therapeutic population;
(d) reducing and/or preventing the suppression of T cell, such as TIL, activity caused by one or more stress; and/or inducing and/or maintaining and/or increasing T cell activity in the presence of one or more stress; and/or increasing T cell survival in response to one or more stress by contacting the third population of TILs with an activator of Nrf2, and
(e) after administering nonmyeloablative lymphodepleting chemotherapy, administering to the mammal the therapeutic population of T cells, wherein the T cells administered to the mammal, whereupon the regression of the cancer in the mammal is promoted.

40. The method according to claim 2, wherein the method is a method for expanding tumor infiltrating lymphocytes (TILs) into a therapeutic population of TILs comprising:
(a) culturing autologous T cells by obtaining a first population of TILs from a tumor resected from a mammal,
(b) performing a first expansion by culturing the first population of TILs in a cell culture medium comprising IL-2;
(c) performing a second expansion by supplementing the cell culture medium of the second population of TILs with additional IL-2, anti-CD3 antibodies, and antigen presenting cells (APCs), to produce a third population of TILs, wherein the third population of TILs is a therapeutic population, and
(d) reducing and/or preventing the suppression of T cell, such as TIL, activity caused by one or more stress; and/or inducing and/or maintaining and/or increasing T cell activity in the presence of one or more stress; and/or increasing T cell survival in response to one or more stress by contacting the third population of TILs with an activator of Nrf2.

41. A method for promoting regression of a cancer in a mammal by expanding tumor infiltrating lymphocytes (TILs) into a therapeutic population of TILs comprising:
(a) culturing autologous T cells by obtaining a first population of TILs from a tumor resected from a mammal,
(b) performing a first expansion by culturing the first population of TILs in a cell culture medium comprising IL-2;
(c) performing a second expansion by supplementing the cell culture medium of the second population of TILs with additional IL-2, anti-CD3 antibodies, and antigen presenting cells (APCs), to produce a third population of TILs, wherein the third population of TILs is a therapeutic population;
(d) reducing and/or preventing the suppression of T cell, such as TIL, activity caused by one or more stress; and/or inducing and/or maintaining and/or increasing T cell activity in the presence of one or more stress; and/or increasing T cell survival in response to one or more stress by contacting the third population of TILs with an activator of Nrf2, and
(e) after administering nonmyeloablative lymphodepleting chemotherapy, administering to the mammal the therapeutic population of T cells, wherein the T cells administered to the mammal, whereupon the regression of the cancer in the mammal is promoted.

42. A method for treating a subject with cancer comprising administering expanded tumor infiltrating lymphocytes (TILs) comprising:
(a) culturing autologous T cells by obtaining a first population of TILs from a tumor resected from a mammal,
(b) performing a first expansion by culturing the first population of TILs in a cell culture medium comprising IL-2;
(c) performing a second expansion by supplementing the cell culture medium of the second population of TILs with additional IL-2, anti-CD3 antibodies, and antigen presenting cells (APCs), to produce a third population of TILs, wherein the third population of TILs is a therapeutic population;
(d) reducing and/or preventing the suppression of T cell, such as TIL, activity caused by one or more stress; and/or inducing and/or maintaining and/or increasing T cell activity in the presence of one or more stress; and/or increasing T cell survival in response to one or more stress by contacting the third population of TILs with an activator of Nrf2, and
(e) after administering nonmyeloablative lymphodepleting chemotherapy, administering to the mammal the therapeutic population of T cells, wherein the T cells administered to the mammal, whereupon the regression of the cancer in the mammal is promoted.

43. A method for expanding tumor infiltrating lymphocytes (TILs) into a therapeutic population of TILs comprising:
(a) culturing autologous T cells by obtaining a first population of TILs from a tumor resected from a mammal,
(b) performing a first expansion by culturing the first population of TILs in a cell culture medium comprising IL-2;
(c) performing a second expansion by supplementing the cell culture medium of the second population of TILs with additional IL-2, anti-CD3 antibodies, and antigen presenting cells (APCs), to produce a third population of TILs, wherein the third population of TILs is a therapeutic population, and
(d) reducing and/or preventing the suppression of T cell, such as TIL, activity caused by one or more stress; and/or inducing and/or maintaining and/or increasing T cell activity in the presence of one or more stress; and/or increasing T cell survival in response to one or more stress by contacting the third population of TILs with an activator of Nrf2.

44. The method according to claim 38, wherein step b) comprises performing a first expansion by culturing the first population of TILs in a cell culture medium comprising IL-2 and one or more TME stimulators to produce a second population of TILs.

45. The method according to claim 2, wherein the one or more T cell and/or NK cell is contacted with an activator of Nrf2 for up to 48 hours, optionally between 0.5 and 24 hours, and preferably for 0.1 hours, 0.2 hours, 0.3 hours, 0.4 hours, 0.5 hours, 0.75 hours, 1 hour, 2 hours, 3 hours, 4 hours, 5 hours, 6 hours, 7 hours, 8 hours, 9 hours, 10 hours, 11 hours, 12 hours, 14 hours, 16 hours, 18 hours or 24 hours.

46. The method according to claim 2, further comprising an optional post-contacting step, wherein the one or more T cell and/or NK cell are incubated in the absence of the activator of Nrf2; optionally wherein the activator of Nrf2 is washed away from the one or more T cell and/or NK cell.

47-52. (canceled)

53. The method according to claim 3, wherein the effect on the one or more T cell and/or NK cell activity and/or survival is present for sufficient time to allow for the treatment and/or prevention of cancer.

54. The method according to claim 2, wherein the effect on T cell and/or NK cell activity and/or survival is present for at least 24 hours, at least 30 hours, at least 36 hours, at least 42 hours, at least 48 hours, at least 54 hours, at least 60 hours, at least 66 hours, and/or at least 72 hours after the contacting step and/or the post-contacting step and/or after the activator of Nrf2 is removed and/or washed away from the one or more T cell and/or NK cell.

55. The method according to claim 2, wherein the effect on T cell and/or NK cell activity and/or survival is reversible and/or is no longer present at least 72 hours, at least 84 hours or at least 96 hours after the contacting step and/or the post-contacting step and/or after the activator of Nrf2 is removed and/or washed away from the one or more T cell and/or NK cell.

56. The method according to claim 2, wherein the cancer is a bladder cancer, bone cancer, breast cancer, colon cancer, cervical cancer, rectal cancer, endometrial cancer, oesophageal cancer, ovarian cancer, gastric cancer, kidney cancer, leukaemia, liver cancer, lung cancer, skin cancer (including basal cell carcinoma, squamous cell carcinoma, melanoma, non-skin located uveal melanoma and/or mucosal melanoma), lymphoma, pancreatic cancer, prostate cancer, testicular cancer and/or thyroid cancer.

57. The method according to claim 2, wherein the cancer is a solid tumour, optionally wherein there is a high level of one or more stress, such as oxidative stress, present in the solid tumour microenvironment.

58. The method according to claim 2, wherein the T cell and/or NK cell is contacted with an activator of Nrf2 at a concentration between 0.1 μM and 500 μM, for example wherein the concentration of the activator of Nrf2 is 0.1 μM, 0.25 μM, 0.5 μM, 0.75 μM, 1 μM, 1.5 μM, 2 μM, 2.5 μM, 3 μM, 4 μM, 5 μM, 7.5 μM, 10 μM, 15 μM, 20 μM, 25 μM, 30 μM, 40 μM, 45 μM, 50 μM, 60 μM, 70 μM, 80 M, 90 μM, 100 μM, 150 μM, 200 μM, 250 μM, 300 M, 350 μM, 400 μM, 450 μM or 500 μM.

59. The method according to claim 2, wherein the T cell and/or NK cell is contacted with an activator of Nrf2 at a concentration between 0.1 μg/ml and 500 μg/ml, for example wherein the concentration of the activator of Nrf2 is 0.1 μg/ml, 0.25 μg/ml, 0.5 μg/ml, 0.75 μg/ml, 1 μg/ml, 1.5 μg/ml, 2 μg/ml, 2.5 μg/ml, 3 μg/ml, 4 μg/ml, 5 μg/ml, 7.5 μg/ml, 10 μg/ml, 15 μg/ml, 20 μg/ml, 25 μg/ml, 30 μg/ml, 40 μg/ml, 45 μg/ml, 50 μg/ml, 60 μg/ml, 70 μg/ml, 80 μg/ml, 90 μg/ml, 100 μg/ml, 150 μg/ml, 200 μg/ml, 250 μg/ml, 300 μg/ml, 350 μg/ml, 400 μg/ml, 450 μg/ml or 500 μg/ml.

60. The method according to claim 2, wherein the activator of Nrf2 is auranofin and wherein the T cell and/or NK cell is contacted with auranofin at a concentration of 1 μM, 5 μM, 10 μM or 25 μM, 0.25 μg/ml, 0.5 μg/ml or 1 μg/ml.

61. The method according to claim 2, wherein the activator of Nrf2 is sulforaphane and wherein the T cell and/or NK cell is contacted with sulforaphane at a concentration of 2.5 μM, 5 μM, 10 μM or 25 M.

62. The method according to claim 2, wherein the activator of Nrf2 is dimethyl fumarate (DMF) and wherein the T cell and/or NK cell is contacted with dimethyl fumarate (DMF) at a concentration of 5 μM, 10 μM or 25 μM.

63. The method according to claim 2, wherein the number of one or more T cell and/or NK cells present is between 1×10⁴ and 1×10⁸ cells, for example 1×10⁴, 1×10⁵, 1×10⁶, 1×10⁷, 1×10⁸ cells, preferably 1×10⁶ cells or 1×10⁷ to 1×10¹² cells, such as 1×10⁸ to 5×10⁹ cells, such as 1×10⁹ to 5×10⁹ cells, such as 1×10⁸ to 5×10¹⁰ cells, such as 1×10⁹ to 5×10¹¹ cells.

64. The method according to claim 2, wherein the one or more T cell is a tumour infiltrating lymphocyte (TIL) and/or a chimeric antigen receptor T cell (CAR-T cell).

65. (canceled)

66. A method for inducing and/or increasing T cell and/or NK cell activity wherein the method comprises a step of contacting one or more T cell and/or NK cell with an activator of Nrf2 ex vivo, wherein the method further comprises a step of administering the one or more T cell and/or NK cell to a patient in need thereof.

67-71. (canceled)
